(19) United States
(12) Patent Application Publication
(10) Pub. No.: US 2020/0010479 A1
(43) Pub. Date: Jan. 9, 2020

(54) INHIBITORS OF MICROBIAL BETA-GLUCURONIDASE ENZYMES AND USES THEREOF

(71) Applicant: THE UNIVERSITY OF NORTH CAROLINA AT CHAPEL HILL, CHAPEL HILL, NC (US)

(72) Inventors: MATTHEW R. REDINBO, DURHAM, NC (US); AADRA BHATT, CARRBORO, NC (US)

(21) Appl. No.: 16/482,998

(22) PCT Filed: Feb. 5, 2018
(86) PCT No.: PCT/IB2018/050717

§ 371 Date: Aug. 1, 2019

Related U.S. Application Data
(60) Provisional application No. 62/454,325, filed on Feb. 3, 2017.

(51) Int. Cl. C07D 495/14; C07D 519/00; A61P 35/00

(52) U.S. Cl. CPC C07D 495/14; A61K 31/437; A61P 35/00; C07D 519/00

(57) ABSTRACT

Methods utilizing compounds and compositions are provided that comprise selective β-glucuronidase inhibitors. The methods can ameliorate the side effects of chemotherapeutic agents and can improve the efficacy of such agents, including irinotecan and non-steroidal anti-inflammatory drugs. The methods comprise administering the compounds in combination with agents or administering the compounds in a monotherapy for the treatment of cancer and gastrointestinal conditions.

### GOVERNMENT SUPPORT

This invention was made with government support under Grants CA098468 and CA207416 awarded by the National Institutes of Health. The government has certain rights in the invention.

### FIELD OF THE INVENTION

The invention relates generally to methods utilizing compounds and compositions that comprise selective β-glucuronidase inhibitors, where selective means inhibitors of bacterial β-glucuronidases but not human or mammalian β-glucuronidases. The methods can ameliorate the side effects of chemotherapeutic agents and can improve the efficacy of such agents, including irinotecan and non-steroidal anti-inflammatory drugs. The methods comprise administering the compounds in combination with agents or administering the compounds in a monotherapy for the treatment of cancer and gastrointestinal conditions.

### BACKGROUND

Irinotecan is a commonly used chemotherapeutic agent used in the treatment of a variety of malignancies. Unfortunately, patients treated with Irinotecan often suffer side effects, such as severe diarrhea, which increases patient suffering and thereby reduces the viability for dose escalation and improved efficacy. Currently, no effective therapy exists to overcome such diarrhea. It can be so severe that it, in fact, becomes a dose limiting side effect.

The underlying mechanism of Irinotecan induced diarrhea has been extensively investigated. The mechanism begins with bacteria β-glucuronidase (GUS) enzymes in the intestines converting the nontoxic metabolite of Irinotecan, SN-38G, to toxic SN-38. The compound SN-38 is the therapeutically active form of Irinotecan, which when present in the intestines, leads to damage of intestinal epithelial cells and diarrhea.

The microbial beta-glucuronidase (GUS) enzymes in the mammalian gastrointestinal (GI) tract are the first established drug targets in the microbiome. GUS enzymes have been shown to reactivate drug metabolites to their toxic forms, which can significantly damage the GI epithelium and reduce drug tolerance and efficacy. (Wallace B. D., et al., Science, 330, 831-835 (2010)).

Certain phyla found in the gut microbiota express β-glucuronidase, an enzyme that can hydrolyze glucuronic acid attached to lipophilic endo- and xenobiotic molecules during Phase II metabolism. Hydrolysis reactivates these previously detoxified molecules, such as the anticancer drug irinotecan, in the gastrointestinal tract, with injurious sequelae such as ulcerations, and bleeding diarrhea.

Compounds and methods that can safely and effectively prevent the reactivation of drug metabolites are needed. The subject matter described herein addresses this need.

### BRIEF SUMMARY

The methods described herein comprise methods for treating a condition or enhancing a chemotherapeutic regimen, comprising administering a compound of Formula I.

In an embodiment, the methods can ameliorate the side effects of chemotherapeutic agents and can improve the efficacy of such agents, including irinotecan and non-steroidal anti-inflammatory drugs. Additionally, the methods may reduce the amount of chemotherapy induced body weight loss. Furthermore, the methods may delay the chemotherapy induced body weight loss.

In an embodiment, the methods comprise administering the compounds in combination with agents or administering the compounds in a monotherapy for the treatment of cancer and gastrointestinal conditions.

In an embodiment, the methods can modulate the amount of gut Proteobacteria. Additionally, the methods may modulate the ratio of gut Proteobacteria to the remainder of gut bacteria.

In an embodiment, the subject matter described herein is directed to methods of treating a condition, such as gastrointestinal distress, which often accompanies treatment of diseases, such as cancer, by administering to a subject in need thereof a compound of Formula I or a pharmaceutical formulation thereof.

Another embodiment includes methods which use pharmaceutical compositions comprising a compound of Formula I or a pharmaceutically acceptable salt and a pharmaceutically acceptable carrier.

Still another embodiment includes methods of inhibiting β-glucuronidases comprising contacting the β-glucuronidase with an effective amount of a compound of Formula I or a pharmaceutically acceptable salt. In a further embodiment, inhibition of β-glucuronidases is selective for bacterial β-glucuronidases over human or other mammalian β-glucuronidases.

Still further embodiments are as described herein.

### BRIEF DESCRIPTION OF THE FIGURES

FIG. 1 shows a 2.3 Å resolution crystal structure of EcGUS revealing that the di-piperazine compound 12 binds in two orientations (A and B) at the GUS active site.

FIG. 2 shows 2.4 Å resolution crystal structure of a microbial GUS in complex with compound 29 and a glucuronic acid from a substrate molecule co-crystalized with enzyme and compound 29.

FIGS. 3A-3C show significant improvement in body weight with Inh9. FIG. 3A shows body weight seven days following treatment. FIGS. 3B and 3C show results at study termination with FIG. 3B showing body weight on sac day and FIG. 3C showing terminal tumor mass.

FIG. 4 shows that irinotecan treatment significantly alters microbiota composition and this effect is diminished by β-glucuronidase Inh9 co-treatment.

FIGS. 5A-5B show Inh9 may have antitumor effects, with FIG. 5A showing tumor volume and FIG. 5B showing body weight.

FIG. 6 shows regions of possible tumors in C3-Tag mouse model (female mice).

FIGS. 7A and 7B show reduced irinotecan-induced diarrhea with Inh9. FIG. 7A shows all mice had diarrhea at day 10. FIG. 7B shows some mice had no diarrhea, but many got diarrhea at days 20-30.

FIG. 8 shows the protection against weight loss with Inh9.

FIGS. 9A and 9B show significant reduction in tumor number and relative mass with Inh9. FIG. 9A shows the tumors per individual mouse. FIG. 9B shows the tumor mass relative to body weight. Both use one way ANOVA, with Sidak's correction for multiple comparisons. No significant difference was observed between other groups. The disclosed result may be indicative of increased tolerability of irinotecan, resulting in increased number of doses, thus a decrease in tumor burden due to irinotecan's antineoplastic effects.

FIGS. 10A and 10B show that the primary and total tumor mass is reduced with Inh9. FIG. 10A shows the primary tumor mass. FIG. 10B shows the total tumor mass. Both use one way ANOVA, with Sidak's correction for multiple comparisons. No significant difference was observed between other groups. The disclosed result may be indicative of increased tolerability of irinotecan, resulting in increased number of doses, thus a decrease in tumor burden due to irinotecan's antineoplastic effects.

FIG. 11 shows the overall increase in survival with irinotecan plus Inh9.

FIGS. 12A-D show that GUS inhibitors inhibit microbial β-glucuronidase via binding the catalytic active site. FIG. 12A shows the structure of GUS Inhibitor 1. FIG. 12B shows the structure of GUS inhibitor compound Inh9 (also referred to herein as GUSi-Inh9 or Inhibitor9), which contains a piperazine moiety. FIG. 12C shows the active site interactions in the Inh9-bound Clostridium perfringens β-glucuronidase. Inh9 (orange) is encompassed by several interacting residues (white) with hydrophobic, electrostatic, and a prominent ring-stacking tyrosine (Tyr472). Water (Wat, red) mediated interactions are located adjacent to the piperazine ring of Inh9, which additionally forms interactions with the two catalytic residues (blue, Glu505 and Glu412). FIG. 12D shows a structural active site superposition of the Inh9-bound Clostridium perfringens β-glucuronidase (light blue) and GUSi-Inh2-bound Escherichia coli β-glucuronidase (light green) crystal structures. Inh9 (orange) and Inh2 (pink) are shown adjacent to the conserved catalytic glutamic acids. The nitrogen in the piperazine group of Inh9 is positioned similarly to the hydroxyethyl group of Inh2, indicating a common need for a proton donor/acceptor group near the catalytic Glu412/Glu413. The polypeptide loops common in some bacterial β-glucuronidase enzymes are shown adjacent to the inhibitors, which make contacts along the loop.

FIGS. 13A-D show gut microbial GUS structure and function in vitro and in fimo. FIG. 13A shows in vitro processing of SN38G by representative members of the main GUS loop topology families demonstrates variability in substrate processing, as well as variable enzymatic inhibition by GUS inhibitors. Error bars are ±SEM, *P<0.05, #P<0.0001 by two-way ANOVA with Dunnett's multiple comparisons test. FIG. 13B shows active site interactions in the Inh9-bound Clostridium perfringes GUS (CpGUS). Inh9 (orange) is encompassed by several interacting residues (white) with hydrophobic, electrostatic, and a prominent ring-stacking tyrosine (Tyr472). Water (Wat, red) mediated interactions are located adjacent to the piperazine ring of Inh9, which additionally forms interactions with the two catalytic residues (blue, Glu505 and Glu412). FIG. 13C shows variable GUS catalytic activity towards SN38G is observed in fimo in wild-type FVB mice. Inh9 diminish in fimo GUS activity to varying degrees. Error bars are ±SEM, ‡P<0.0001, **P<0.01, *P<0.05 by two-way ANOVA with Dunnett's multiple comparisons test. FIG. 13D shows elevated in fimo GUS activity observed in animals pretreated with irinotecan 24 hours prior; co-treatment with Inh9 blunts the increase. Error bars are ±SEM. *P<0.05 by one-way ANOVA with Sidak's correction for multiple comparisons.

FIGS. 14A-E show variable GUS catalytic activity towards SN38G is observed in fimo in (FIG. 14A) proximal and (FIG. 14B) distal in wild-type FVB mice sourced from different cages. Inh9 diminish in fimo GUS activity to varying degrees. Error bars are ±SEM, *P<0.0001, **P<0.01, *P<0.05 by two-way ANOVA with Dunnett's multiple comparisons test. Inh9 reduces in fimo GUS activity from cecal contents in a dose-dependent manner. The graphs in FIGS. 14C-E show apparent k_{cat} values for individual wild-type FVB mice sourced from different cages.

FIGS. 15A-E show that Inh9 (i.e., GUSi) reduces the acute toxicity exerted by irinotecan (IRI) treatment. FIG. 15A shows single IRI injection induces modest body mass reduction over five days of treatment. FIG. 15B shows spleen mass remains unchanged following IRI treatment. FIG. 15C shows Inh9 co-treatment significantly improves hematologic markers in IRI-treated mice. *P<0.05 by unpaired two-tailed t test. FIG. 15D shows larger numbers of apoptotic cells observed in crypts in duodenum of IRI-treated mice, compared to IRI+Inh9. Other small intestinal regions show trends in increased apoptotic cells with IRI treatment. ***P<0.001, two-way ANOVA with Sidak's multiple comparison test. Mice were injected with BrdU for 30 min prior to euthanasia to assess proliferating intestinal cells; the numbers of BrdU+ cells in ten consecutive crypts were blindly quantified in the FIG. 15E ileum; FIG. 15F proximal colon; FIG. 15G distal colon. For FIGS. 15E, F, and G: *P<0.05, **P<0.01 by one-way ANOVA with Sidak's multiple comparisons test. Error bars are ±SEM. Immunohistochemistry to detect BrdU+ cells (brown) in FIG. 15E ileum; FIG. 15F proximal; and FIG. 15G distal halves of colons of mice treated as indicated; nuclei are counterstained with hematoxylin (blue). Scale bar, 50 μm.

FIGS. 16A-H show that Inh9 (GUSi) modestly reduces the acute toxicity 24 hours after single IRI injection. No difference in (FIG. 16A) body mass, (FIG. 16B) normalized spleen mass, or (FIG. 16C) hematocrit 24 hours following single IRI injection. FIG. 16D shows that larger numbers of apoptotic cells observed in crypts in jejunum of IRI-treated mice, compared to IRI+Inh9. Other small intestinal regions show trends in increased apoptotic cells with IRI treatment. **P<0.01, two-way ANOVA with Sidak's multiple comparison test. Mice were injected with BrdU for 30 min prior to euthanasia to assess proliferating intestinal cells. The numbers of BrdU+ cells in ten consecutive crypts were blindly quantified in the (FIG. 16E) ileum; (FIG. 16F) proximal colon; and (FIG. 16G) distal colon; no differences were observed. FIG. 16H shows the immunohistochemistry to detect BrdU+ cells (brown) in distal colons of mice treated as indicated; nuclei are counterstained with hematoxylin (blue).

FIGS. 17A-E show Inh9 maintains irinotecan efficacy in tumor xenograft model. FIG. 17A shows Kaplan-Meier analysis of proportions of animals remaining diarrhea-free with Inh9 cotreatment with irinotecan. *P<0.05 by Log-Rank (Mantel-Cox) test. FIG. 17B shows percent change in body weight of athymic nude mice xenografted with Sum149 cells throughout treatment course. **P<0.01, ***P<0.001 by one-way ANOVA (Sidak multiple comparison test). No significant changes were observed in dual-treated mice compared to controls. FIG. 17C shows Inh9 cotreatment results in significant tumor regression to levels similar to single agent irinotecan. ***P<0.001 by one-way ANOVA (Dunnett multiple comparison test to vehicle treatment). FIG. 17D shows Inh9 cotreatment does not impede the antitumor efficacy of irinotecan as evidenced by terminal tumor mass. ***P<0.001 by one-way ANOVA (Sidak multiple comparison test). FIG. 17E shows epithelial erosions, loss of glandular structures, collapsed goblet cells, and increased inflammatory infiltrates in colons of IRI-treated mice; Inh9 co-treatment preserves intestinal architecture. Black scale bar=37.5 μm.

FIGS. 18A and 18B show (FIG. 18A) IRI+Inh9 treated athymic nude mice have a slightly improved overall survival compared to IRI treatment alone. ns, not significant. FIG. 18B shows that co-treatment with Inh9 allows athymic nude mice to tolerate higher number of IRI doses, compared to IRI treatment alone.

FIGS. 19A-G show that Inh9 improves irinotecan efficacy in C3Tag breast cancer GEMM. FIG. 19A shows Kaplan-Meier analysis of proportions of animals remaining diarrhea-free with Inh9 cotreatment with irinotecan. *P<0.05 by Log-Rank (Mantel-Cox) test. FIG. 19B shows percent change in body weight of C3Tag GEMM mice throughout treatment course. § single animal remaining at this time point. FIG. 19C shows Inh9 co-treatment extends the overall survival of irinotecan-treated mice by 14 days. *P<0.05 by Log-Rank (Mantel-Cox) test. FIG. 19D shows IRI reduces tumor masses in C3Tag animals, compared to vehicle or Inh9 treatment. IRI+Inh9 significantly diminishes tumor masses compared to IRI alone. **P<0.01 by one-way ANOVA with Sidak's multiple comparisons test. FIG. 19E shows normalizing tumor mass to the corresponding body mass indicates that tumors comprise a greater fraction of body weight in control animals compared to IRI treated animals; IRI+Inh9 treated mice have a significantly lower ratio of tumor: body mass compared to IRI alone, reflective of the significantly improved body weight in FIG. 19B and smaller tumor masses in FIG. 19D. **P<0.01 by one-way ANOVA with Sidak's multiple comparisons test. FIG. 19F shows smoothed curves of tumor volumes (statistical methods described in Supplemental Information) indicate that compared to vehicle or Inh9 alone, IRI and IRI+Inh9 significantly (*P<0.05, ***P<0.001 respectively by one-way ANOVA with Sidak's multiple comparisons test) reduce tumor volumes. Regression analysis reveals no significant differences in tumor volumes between IRI and IRI+Inh9, confirming that Inh9 cotreatment does not affect the antitumor activity of IRI. FIG. 19G shows H&E stained colon tissue show well maintained epithelium with intact goblet cells n vehicle and Inh9 treated mice. Erosions, abscessed crypts, inflammatory infiltrates and collapsed mucus glands are evident in IRI mice. Inh9 cotreatment shows infrequent abscesses, with the entire epithelium showing signs of regeneration.

FIG. 20 shows the schematic of IRI and Inh9 dosing schedule in C3Tag transgenic mice. Shaded boxes are days on which drugs were administered, IRI by intraperitoneal injection, and Inh9 by oral gavage. M=Monday, T=Tuesday, W=Wednesday, Th=Thursday, F=Friday, Sa=Saturday, Su=Sunday.

FIGS. 21A and 21B show that (FIG. 21A) regardless of treatment, C3Tag mice have similar number of tumors upon initiation of treatment. Tumor number reflects the primary tumor as well as at secondary sites as previously reported by Green, et al. Oncogene, 2000. FIG. 21B shows that with Inh9 co-treatment, IRI-treated mice tolerate a significantly higher number of IRI doses compared to IRI alone. *P<0.05 by one-way ANOVA with Dunnett's correction for multiple comparisons.

FIGS. 22A-D show how Irinotecan changes gut microbial composition in two mouse models. FIG. 22A shows Chao1 richness of colon contents of treatment groups of the athymic Sum149 tumor xenograft mice as assessed by 16S rRNA metataxonomic sequencing. FIG. 22B shows the phylum composition of colon contents of athymic Sum149 tumor xenograft treatment groups showing marked changes induced by irinotecan and blunted by Inh9. FIG. 22C shows PCoA of colon contents of C3Tag immune-competent GEMM animals by treatment groups showing that irinotecan, but not Inh9, impacts gut microbial composition. FIG. 22D shows that Irinotecan induces increases in levels of Gammaproteobacteria class taxa and Verrocomicrobiaceae class taxa, specifically Akkermansia mucinophila.

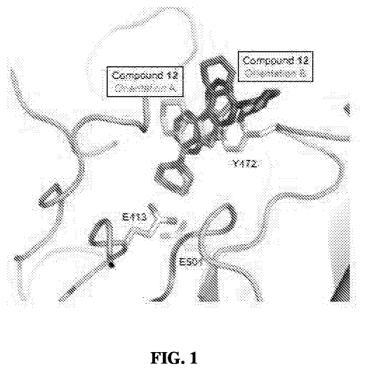

### DETAILED DESCRIPTION

The selectively inhibiting agents of Formula I and compositions thereof as disclosed herein can be used in connection with methods for treating cancer and for reducing side effects of antineoplastic agents, such as camptothecin-derived antineoplastic agents. The gastrointestinal distress that typically accompanies treatment with a particular chemotherapeutic agent can be attenuated or ameliorated. The methods are also useful for attenuating or improving any adverse reactions associated with administration of glucuronidase-substrate agent(s) or compound(s). The subject matter described herein includes compositions and methods for inhibiting bacterial β-glucuronidases and for improving efficacy of camptothecin-derived antineoplastic agents or glucuronidase-substrate agents or compounds by attenuating the gastrointestinal distress caused by reactivation of glucuronidated metabolites of such agents.

The compounds of Formula I can have anti-tumor properties. That is, the compounds described herein have been shown to decrease tumor volume as compared to control. As such, the compounds disclosed herein can be effective monotherapies for the treatment of cancer.

Additionally, the compounds of Formula I can modulate the gut microbiota. That is, the compounds described herein have been shown to reduce the amount of gut Proteobacteria or to modulate the ratio of gut Proteobacteria to the remainder of gut bacteria.

Furthermore, the compounds of Formula I can improve the efficacy of chemotherapeutic agents.

It has been found that inhibition of microbial beta-glucuronidase (GUS) can alleviate the side effects and toxicity of anticancer drugs, such as irinotecan, as well as several non-steroidal anti-inflammatory drugs. Successful inhibitors of microbial GUS enzymes have the following three characteristics: potency, non-lethality to bacterial and mammalian cells, and selectivity for the microbial GUS enzymes over the mammalian GUS proteins. (Wallace, (2010)). As used herein, “beta-glucuronidase,” “β-glucuronidase” and the like means an enzyme (EC 3.2.1.31) capable of hydrolyzing β-glucuronides, but not α-glucuronides or β-glucosides. (Basinska & Florianczyk (2003) Ann. Univ. Mariae Curie Sklodowska Med. 58:386-389; Miles et al. (1955) J. Biol. Chem. 217:921-930). As used herein, a “glucuronide” and the like means a substance produced by linking glucuronic acid to another substance via a glycosidic bond. Examples of glucuronides of interest herein include, but are not limited to, glucuronides of camptothecin-derived antineoplastic agents such as SN-38G (7-ethyl-10-hydroxycamptothecin glucuronide). Further details of microbiota beta-glucuronidase is disclosed in U.S. Pat. No. 8,557,808, herein incorporated by reference in its entirety.

Reactivation of inactive metabolites such as SN-38G to active SN-38 occurs in the gastrointestinal tract and results from bacterial β-glucuronidases. The reactivated metabolites can lead to a gastrointestinal distress such as diarrhea, which often can be a dose-limiting side effect of the cancer therapy or the therapy to treat any other conditions.

As used herein, “dose-limiting” indicates that the side effect from administration of a camptothecin-derived antineoplastic agent or glucuronidase-substrate agents or compounds prevents a subject in need of cancer therapy or therapy to treat any other conditions from receiving a recommended amount. As increasing amounts of the camptothecin-derived antineoplastic agent or glucuronidase-substrate agents or compounds are administered to a subject, increased amounts of glucuronidated metabolites are therefore available as a substrate for the bacterial β-glucuronidases. The resulting reactivated metabolites not only adversely affect a subject's well-being by causing serious side effects, particularly gastrointestinal distress, but also impair treatment outcome by limiting the amount of the camptothecin-derived antineoplastic agent or glucuronidase-substrate agents or compounds that can be administered to the subject.

As used herein, “serious side effects” includes side effects such as gastrointestinal distress, diarrhea, localized inflammation, intestinal epithelial crypt damage (erosion, cell death), golet cell damage, mucositis, and regeneration that causes discontinuation of the treatment, such as irinotecan therapy.

While not being bound to theory, it is believed that the compounds described herein are inhibitors of hydrolysis of a covalent sugar-enzyme intermediate of the glycosyl hydrolase reaction mechanism. As described herein, the compounds and methods are selective for the bacterial microbiome that re-activates drugs in the gut. Accordingly, the compounds and methods below are useful for ameliorating the side effects of many drugs and treatments. Specifically, described herein is the use of compounds of Formula I that inhibit enzymes in the GI microbiota, and that such compounds significantly improve the treatment of cancer with an antineoplastic compound, such as irinotecan.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Definitions

The term “substituent” refers to an atom or a group of atoms that replaces a hydrogen atom on a molecule. The term “substituted” denotes that a specified molecule bears one or more substituents. The term “a compound of the formula” or “a compound of formula” or “compounds of the formula” or “compounds of formula” refers to any compound selected from the genus of compounds as defined by Formula I.

As used herein, the term “alkyl” refers to a straight-chained or branched hydrocarbon group. Examples of alkyl groups include methyl, ethyl, n-propyl, isopropyl, tert-butyl, and n-pentyl. Alkyl groups may be optionally substituted with one or more substituents.

The term “benzyl” refers to a hydrocarbon with the formula of C₆H₅CH₂ where the point of attachment to the group in question is at the CH₂ position. The benzyl may be substituted on the aromatic ring. In one embodiment, 0, 1, 2, 3, 4, or 5 atoms of the aryl group may be substituted by a substituent.

As used herein, the term “halogen”, “hal” or “halo” means —F, —Cl, —Br or —I.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, furanyl, thienyl, pyrrolyl, oxazolyl, oxadiazolyl, imidazolyl, thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “nitrogen-containing heteroaryl” refers to a heteroaryl group having 1-4 ring nitrogen heteroatoms if monocyclic, 1-6 ring nitrogen heteroatoms if bicyclic, or 1-9 ring nitrogen heteroatoms if tricyclic.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolanyl, tetrahydrofuryl, tetrahydrothienyl, thienyl, and the like.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not minor images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

Furthermore the compounds described herein may include olefins having either geometry: “Z” refers to what is referred to as a “cis” (same side) configuration whereas “E” refers to what is referred to as a “trans” (opposite side) configuration.

A “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

A “metabolite” is a product produced through metabolism in the body of camptothecin-derived antineoplastic agents or glucuronidase-substrate agents or compounds. Metabolites may be identified using routine techniques known in the art and their activities determined using tests such as those described herein. Such products may result, for example, from the oxidation, hydroxylation, reduction, hydrolysis, amidation, deamidation, esterification, deesterification, enzymatic cleavage, and the like, of the administered compound.

The phrase “pharmaceutically acceptable” indicates that the substance or composition must be compatible chemically and/or toxicologically, with the other ingredients comprising a formulation, and/or the subject being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt, the salt can have multiple counter ions. Hence, a pharmaceutically acceptable salt can have one or more charged atoms and/or one or more counter ion.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. In certain embodiments, the pharmaceutically acceptable carrier is a non-naturally occurring pharmaceutically acceptable carrier.

Use of the word “inhibitor” herein is meant to mean a molecule that inhibits activity of microbial GUS enzymes. By “inhibit” herein is meant to decrease the activity of the target enzyme, as compared to the activity of that enzyme in the absence of the inhibitor. In some embodiments, the term “inhibit” means a decrease in GUS activity of at least about 5%, at least about 10%, at least about 20%, at least about 25%, at least about 50%, at least about 60%, at least about 70%, at least about 80%, at least about 90%, or at least about 95%. In other embodiments, inhibit means a decrease in GUS activity of about 5% to about 25%, about 25% to about 50%, about 50% to about 75%, or about 75% to 100%. In some embodiments, inhibit means a decrease in GUS activity of about 95% to 100%, e.g., a decrease in activity of 95%, 96%, 97%, 98%, 99%, or 100%. Such decreases can be measured using a variety of techniques that would be recognizable by one of skill in the art, including in vitro assays.

As used herein, a “beta-glucuronidase inhibitor,” “GUS inhibitor” or an “inhibitor of GUS” is a compound that reduces, inhibits, or otherwise diminishes one or more of the biological activities of microbial GUS enzyme(s). Inhibition does not necessarily indicate a total elimination of GUS activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of GUS compared to an appropriate control. In some embodiments, the compounds disclosed herein reduce, inhibit, or otherwise diminish microbial GUS enzyme activity.

By “specific inhibition” is intended an agent that reduces, inhibits, or otherwise diminishes the activity of a specific microbial GUS enzyme or ortholog greater than that of another microbial GUS enzyme or ortholog. For example, a specific inhibitor reduces at least one biological activity of a GUS ortholog, e.g., E. coli GUS (EcGUS) by an amount that is statistically greater than the inhibitory effect of the compound on another GUS ortholog, e.g., Streptococcus agalactiae (SaGUS) or Clostridium perfringens (CpGUS). In some embodiments, the IC₅₀ of the inhibitor for an ortholog is about 90%, 80%, 70%, 60%, 50%, 40%, 30%, 20%, 10%, 5%, 1%, 0.1%, 0.01%, 0.001% or less of the IC₅₀ of the inhibitor for another ortholog. The presently disclosed compounds may or may not be a specific inhibitor. A specific inhibitor reduces the biological activity of a particular GUS by an amount that is statistically greater than the inhibitory effect of the inhibitor on any other GUS.

As used herein, “selectively inhibit” and the like means that a β-glucuronidase inhibitor reduces bacterial, but not mammalian, β-glucuronidase activity. That is, the β-glucuronidase inhibitor can bind to and can prevent bacterial, but not mammalian, β-glucuronidases from hydrolyzing glucuronides. Useful compounds of Formula I are selective for bacterial β-glucuronidase. That is, the compounds decrease activity of the bacterial β-glucuronidase by a statistically significant amount as compared to the mammalian β-glucuronidase. Advantageously, the β-glucuronidase inhibitors described herein are selective for bacterial β-glucuronidases. That is, the compounds inhibit β-glucuronidase in bacteria but do not have inhibitory activity toward mammalian β-glucuronidases, including human β-glucuronidase. While not intending to be bound by any particular mechanism of action, the compounds appear to bind a \tilde{ }12 residue loop in bacterial β-glucuronidases that hovers over an active site opening. The loop is not present in mammalian β-glucuronidases, which therefore can accommodate larger substrates and cleave glucuronic acid moieties from long-chain glycosaminoglycans. The β-glucuronidase inhibitors exhibit other advantages. For example, the compounds do not kill the enteric bacteria or harm human epithelial cells, but are effective against bacteria cultured under aerobic and anaerobic conditions.

As used herein, “enteric bacteria” and the like mean the normal bacteria that inhabit the human gastrointestinal tract. Examples of enteric bacteria include, but are not limited to, Bacteroides sp. (e.g., Bacteroides vulgatus), Bifidobacterium sp. (e.g., Bifidobacterium bifidum and Bifidobacterium infantis), Catenabacterium sp., Clostridium sp., Corynebacterium sp., Enterococcus sp. (e.g., Enterococcus faecalis), Enterobacteriaceae (e.g., Escherichia coli), Lactobacillus sp., Peptostreptococcus sp., Propionibacterium sp., Proteus sp., Mycobacterium sp., Pseudomonas sp. (e.g., Pseudomonas aeruginosa), Staphylococcus sp. (e.g., Staphylococcus epidermidis and Staphylococcus aureus) and Streptococcus sp. (e.g., Streptococcus mitis). Because enteric bacteria commensally inhabit the gastrointestinal tract, they promote gastrointestinal health by preventing infection by opportunistic bacteria like Clostridium difficle.

The terms “treat” and “treatment” refer to both therapeutic treatment and prophylactic or preventative measures, wherein the object is to prevent or slow down (lessen) an undesired physiological change or disorder. The terms “alleviate” and “attenuate” refer to the degree and rate of occurrence of the objective and subjective symptomology associated with GI distress. Beneficial or desired clinical results include, but are not limited to, alleviation of symptoms, diminishment of extent of conditions, stabilized (i.e., not worsening) condition, delay or slowing of side effects, lessening of GI distress relative to the GI distress suffered prior to receiving a compound of Formula I. “Treatment” can also mean improved side effects as compared to expected side effects if not receiving treatment. Those in need of treatment include those already with the condition or disorder as well as those prone to have the condition or disorder or those in which the condition or disorder is to be prevented.

As used herein, the terms “reduce” and “reducing” means to lessen. That is, for example, symptoms associated with gastrointestinal distress may be reduced. Likewise, the side effects of a chemotherapeutic agent can be reduced. Thus, to reduce means to lessen of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or up to about 100% as compared to an appropriate control.

As used herein, the terms “GI distress” or “gastrointestinal distress” refers to symptoms associated with chemotherapy-induced gastrointestinal conditions or conditions associated with the presence of proteobacteria in the GI tract. Such conditions include, but are not limited to, severe diarrhea. Still other conditions include but are not limited to: localized inflammation, intestinal epithelial crypt damage (erosion, cell death), golet cell damage, mucositis, and regeneration.

The term “administration” or “administering” includes routes of introducing the compound(s) to a subject to perform their intended function.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the inhibitor compound are outweighed by the therapeutically beneficial effects.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “subject” refers to animals such as mammals, including, but not limited to, primates (e.g., humans), cows, sheep, goats, horses, dogs, cats, rabbits, rats, mice and the like. In certain embodiments, the subject is a human.

Additional definitions are also provided below.

### II. Methods

The methods described herein comprise methods for treating a condition or enhancing a chemotherapeutic regimen, comprising administering a compound of Formula I, which also includes pharmaceutically acceptable salts, having the following structure:
wherein,

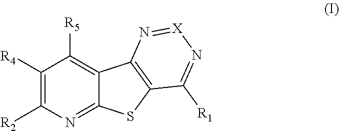

R₁ is selected from the group consisting of:
i. —OR_{A}, wherein R_{A} is selected from the group consisting of H, CF₃, optionally substituted linear or branched C₂₋₆ alkyl, optionally substituted benzyl, and C₃₋₈ cycloalkyl;
ii. —NR_{B}R_{C}, wherein R_{B} and R_{C} are each independently selected from the group consisting of H, linear or branched C₁₋₆ alkyl optionally substituted with amino, optionally substituted benzyl, C₂₋₅ heteroaryl, C₂₋₅ heteroalkyl, and C₃₋₈ cycloalkyl;
iii. C₂₋₅ heteroaryl optionally substituted with hydroxyl, halo, or amino;
iii. —N(CH₂)ₚ—Z, wherein p is 1 or 2; and Z is heteroaryl or heterocycloalkyl optionally substituted with hydroxyl, halo, or amino;
iv.

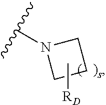

wherein s is 1, 2, or 3; and R_{D} is amino, hydroxyl, halo, or linear or branched C₁₋₆ alkyl; and
v.

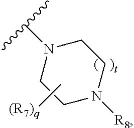

wherein
   R₇ is H, linear or branched C₁₋₆ alkyl optionally substituted with halo, amino, or hydroxyl;
   t is 1 or 2;
   q is 1 or 2, wherein R₇ can join form a bridge; and
   R₈ is H, linear or branched C₁₋₄ alkyl optionally substituted with halo, hydroxyl, or amino;

R₂ is H, aryl, heterocycloalkyl, or —NR_{F}R_{G}, wherein
R_{F} and R_{G} are independently selected from the group consisting of H, linear or branched C₁₋₆ alkyl optionally substituted with halo, amino or hydroxyl, optionally substituted benzyl, and C₃₋₈ cycloalkyl;

X is N or —CR₃, wherein
R₃ is selected from the group consisting of H, halogen, —OR_{I}, and —NR_{I}R_{J}, wherein
   R_{I} and R_{J} are each independently selected from H and linear or branched C₁₋₆ alkyl, wherein the alkyl can be optionally substituted with hydroxyl; and

R₄ and R₅ are H or are taken together with the carbon to which each is attached to form an optionally substituted C₅₋₇ membered ring.

In embodiments, R₄ and R₅ are taken together with the carbon to which each is attached to form an optionally substituted C₅₋₇ membered ring.

In any above embodiment, X is N.

In any above embodiment, R₂ is heterocycloalkyl.

In any above embodiment, R₂ is a 4-8 membered ring containing 1 or 2 nitrogen atoms.

In any above embodiment, R₂ is chosen from the group consisting of:

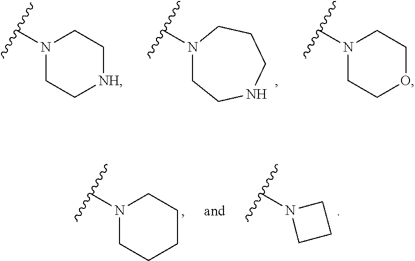 [SMILES: *N1CCNCC1 ; *N1CCCNCC1 ; *N1CCOCC1 ; *N1CCCCC1 ; *N1CCC1]

In any above embodiment, R₂ is morpholinyl.

In any above embodiment, R₁ is
—NR_{B}R_{C}, wherein R_{B} and R_{C} are each independently selected from the group consisting of H, linear or branched C₁₋₆ alkyl optionally substituted with amino, optionally substituted benzyl, C₂₋₅ heteroaryl, C₂₋₅ heteroalkyl, and C₃₋₈ cycloalkyl;
R₁ is C₂₋₅ heteroaryl optionally substituted with hydroxyl, halo, or amino; or,
R₁ is

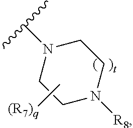

wherein
   R₇ is H, linear or branched C₁₋₆ alkyl optionally substituted with halo, amino, or hydroxyl;
   t is 1 or 2;
   q is 1 or 2 and when q is 2 the two R₇ groups may join to form a bridged compound; and
R₈ is H, linear or branched C₁₋₄ alkyl optionally substituted with halo, hydroxyl, or amino.

In any above embodiment, R₁ is

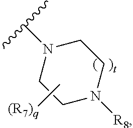

wherein R₇ is H, linear or branched C₁₋₆ alkyl optionally substituted with halo, amino, or hydroxyl;
t is 1 or 2;
q is 1 or 2, wherein R₇ can join form a bridge; and
R₈ is H, linear or branched C₁₋₄ alkyl optionally substituted with halo, hydroxyl, or amino.

In any above embodiment, t and q are each 1.

In any above embodiment, R₈ is H.

In any above embodiment, R₇ is methyl.

In any above embodiment, R₁ is

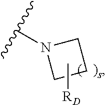

wherein
   s is 1, 2, or 3; and
   R_{D} is amino, hydroxyl, halo, or linear or branched C₁₋₆ alkyl;

or
R₁ is

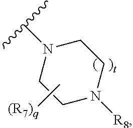

wherein
   R₇ is H, linear or branched C₁₋₆ alkyl optionally substituted with halo, amino, or hydroxyl;
   t is 1 or 2;
   q is 1 or 2, wherein R₇ can join form a bridge; and
   R₈ is H, linear or branched C₁₋₄ alkyl optionally substituted with halo, hydroxyl, or amino.

In any above embodiment, R₁ is particularly

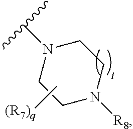

wherein
R₇ is H, linear or branched C₁₋₆ alkyl optionally substituted with halo, amino, or hydroxyl;
t is 1 or 2;
q is 1 or 2, wherein R₇ can join form a bridge; and
R₈ is H, linear or branched C₁₋₄ alkyl optionally substituted with halo, hydroxyl, or amino.

In any above embodiment, t and q are each 1.

In any above embodiment, R₇ is H.

In any above embodiment, R₈ is H.

In any above embodiment, R₂ is H, heterocycloalkyl, or —NR_{F}R_{G}, wherein
R_{F} and R_{G} are independently selected from the group consisting of H, linear or branched C₁₋₆ alkyl optionally substituted with halo, amino or hydroxyl, optionally substituted benzyl, and C₃₋₈ cycloalkyl.

In any above embodiment, R₂ is heterocycloalkyl.

As in any above embodiment, the compound of Formula I is

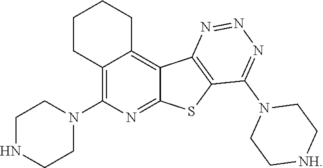 [SMILES: C1CCc2c(c(N3CCNCC3)nc3sc4c(N5CCNCC5)nnnc4c23)C1]

As in any above embodiment, the compound of Formula I is

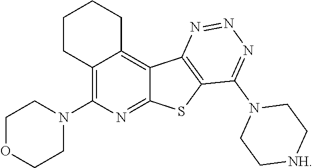 [SMILES: C1CCc2c(c(N3CCOCC3)nc3sc4c(N5CCNCC5)nnnc4c23)C1]

In any above embodiment, R₂ is —NR_{F}R_{G}, wherein
R_{F} and R_{G} are independently selected from the group consisting of H, linear or branched C₁₋₆ alkyl optionally substituted with halo, amino or hydroxyl, optionally substituted benzyl, and C₃₋₈ cycloalkyl.

In any above embodiment, R_{F} and R_{G} are independently selected from H or linear or branched C₁₋₆ alkyl optionally substituted with halo, amino or hydroxyl.

In a particular embodiment, the compound of Formula I is

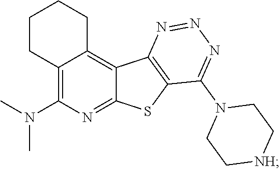 [SMILES: CN(C)c1nc2sc3c(N4CCNCC4)nnnc3c2c2c1CCCC2]

or,

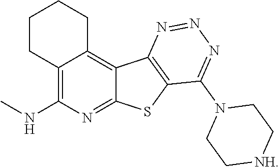 [SMILES: CNc1nc2sc3c(N4CCNCC4)nnnc3c2c2c1CCCC2]

In any above embodiment, wherein R₄ and R₅ are H.

In any above embodiment, R₁ is

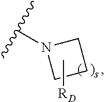

wherein
   s is 1, 2, or 3; and
   R_{D} is amino, hydroxyl, halo, or linear or branched C₁₋₆ alkyl;
or
R₁ is

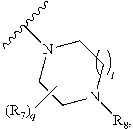

wherein
R₇ is H, linear or branched C₁₋₆ alkyl optionally substituted with halo, amino, or hydroxyl;
t is 1 or 2;
q is 1 or 2, wherein R₇ can join form a bridge; and
R₈ is H, linear or branched C₁₋₄ alkyl optionally substituted with halo, hydroxyl, or amino.

In any above embodiment, R₁ is

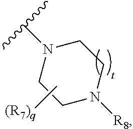

wherein
R₇ is H, linear or branched C₁₋₆ alkyl optionally substituted with halo, amino, or hydroxyl;
t is 1 or 2;
q is 1 or 2, wherein R₇ can join form a bridge; and
R₈ is H, linear or branched C₁₋₄ alkyl optionally substituted with halo, hydroxyl, or amino.

In any above embodiment, t and q are each 1.

In any above embodiment, R₇ is H.

In any above embodiment, R₁ is piperazinyl.

In any above embodiment, R₂ is H, aryl, heterocycloalkyl, or —NR_{F}R_{G}, wherein
R_{F} and R_{G} are independently selected from the group consisting of H, linear or branched C₁₋₆ alkyl, optionally substituted benzyl, and C₃₋₈ cycloalkyl.

In any above embodiment, R₂ is aryl.

In any above embodiment, R₂ is phenyl.

In any above embodiment, R₂ is heterocycloalkyl.

In any above embodiment, R₂ is a 4-8 membered ring containing 1 or 2 nitrogen atoms contained within the ring.

In any above embodiment, R₂ is chosen from the group consisting of:

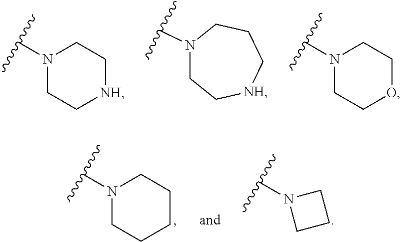 [SMILES: *N1CCNCC1 ; *N1CCCNCC1 ; *N1CCOCC1 ; *N1CCCCC1 ; *N1CCC1]

In any above embodiment, R₂ is morpholinyl.

In any above embodiment, R₂ is —NR_{F}R_{G}, wherein
R_{F} and R_{G} are independently selected from the group consisting of H, linear or branched C₁₋₆ alkyl optionally substituted with halo, amino or hydroxyl, optionally substituted benzyl, and C₃₋₈ cycloalkyl.

In any above embodiment, R_{F} and R_{G} are independently H or linear or branched C₁₋₆ alkyl optionally substituted with halo, amino or hydroxyl.

In any above embodiment, R_{F} and R_{G} are independently H or unsubstituted linear C₁₋₆ alkyl.

In a particular embodiment, the compound of Formula I is

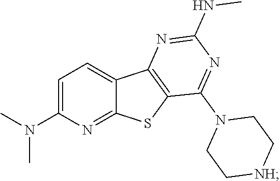 [SMILES: CNc1nc(N2CCNCC2)c2sc3nc(N(C)C)ccc3c2n1]

or,

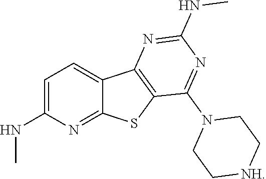 [SMILES: CNc1ccc2c(n1)sc1c(N3CCNCC3)nc(NC)nc12]

As in any above, the compound of Formula I is selected from the group consisting of:

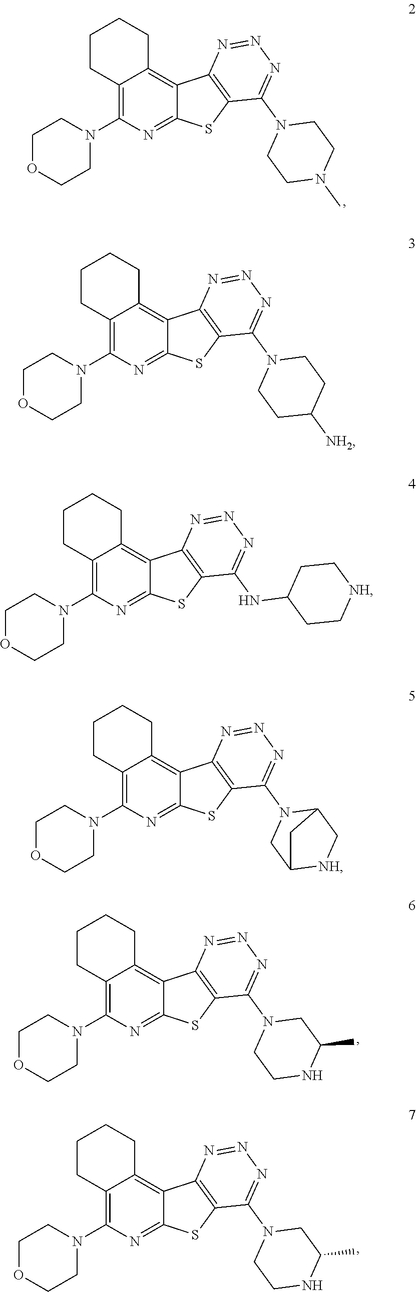 [SMILES: CN1CCN(c2nnnc3c2sc2nc(N4CCOCC4)c4c(c23)CCCC4)CC1 ; NC1CCN(c2nnnc3c2sc2nc(N4CCOCC4)c4c(c23)CCCC4)CC1 ; C1CCc2c(c(N3CCOCC3)nc3sc4c(NC5CCNCC5)nnnc4c23)C1 ; C1CCc2c(c(N3CCOCC3)nc3sc4c(N5CC6CC5CN6)nnnc4c23)C1 ; C[C@@H]1CN(c2nnnc3c2sc2nc(N4CCOCC4)c4c(c23)CCCC4)CCN1 ; C[C@H]1CN(c2nnnc3c2sc2nc(N4CCOCC4)c4c(c23)CCCC4)CCN1]

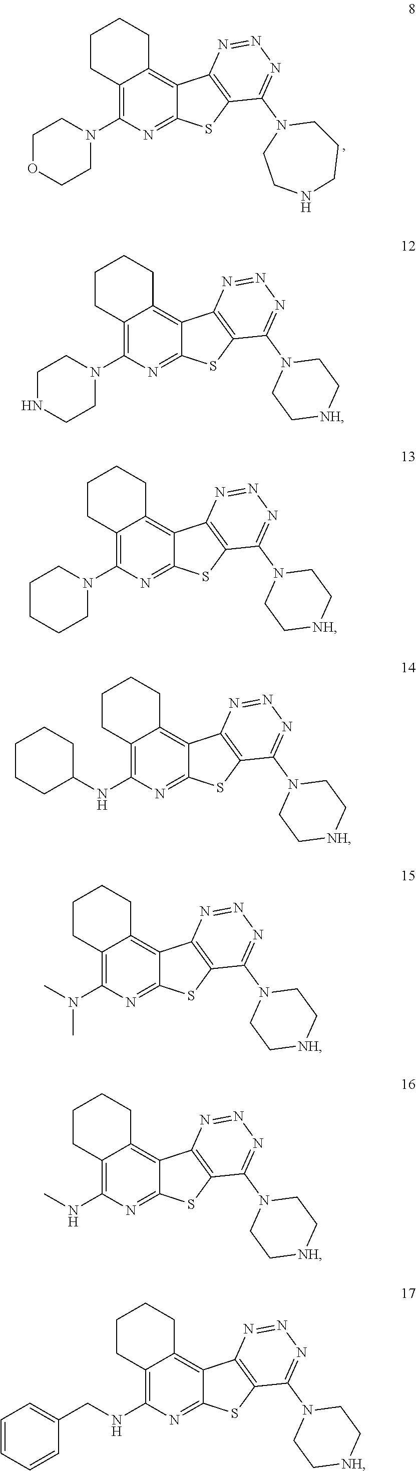

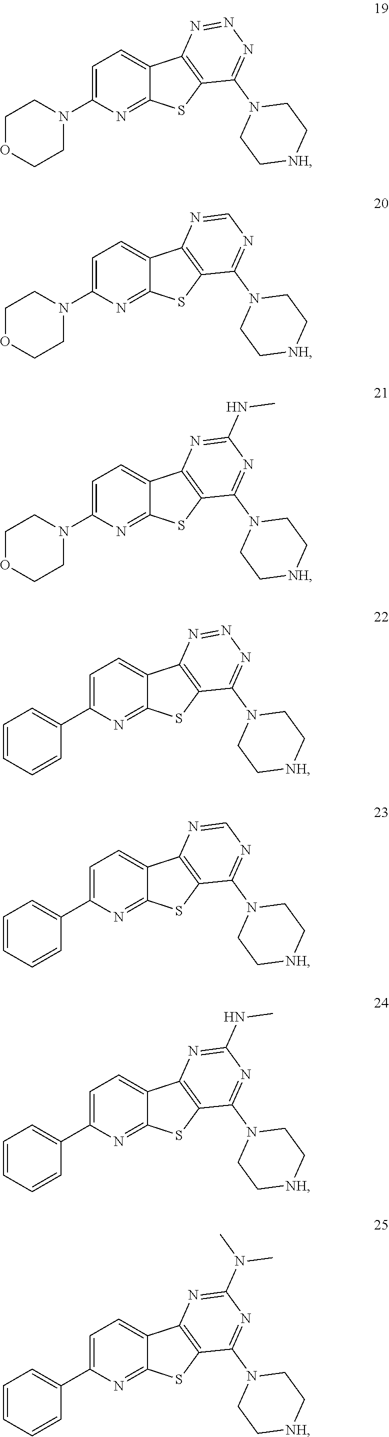

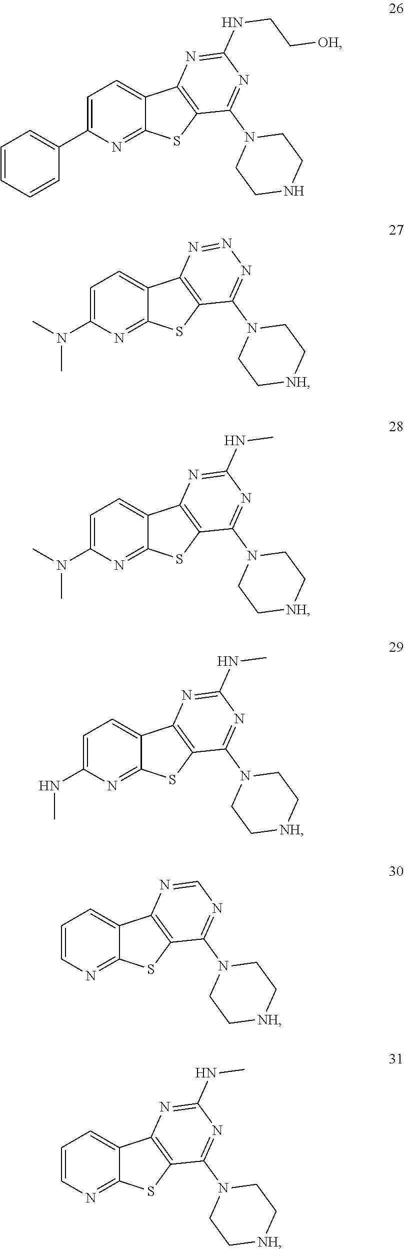

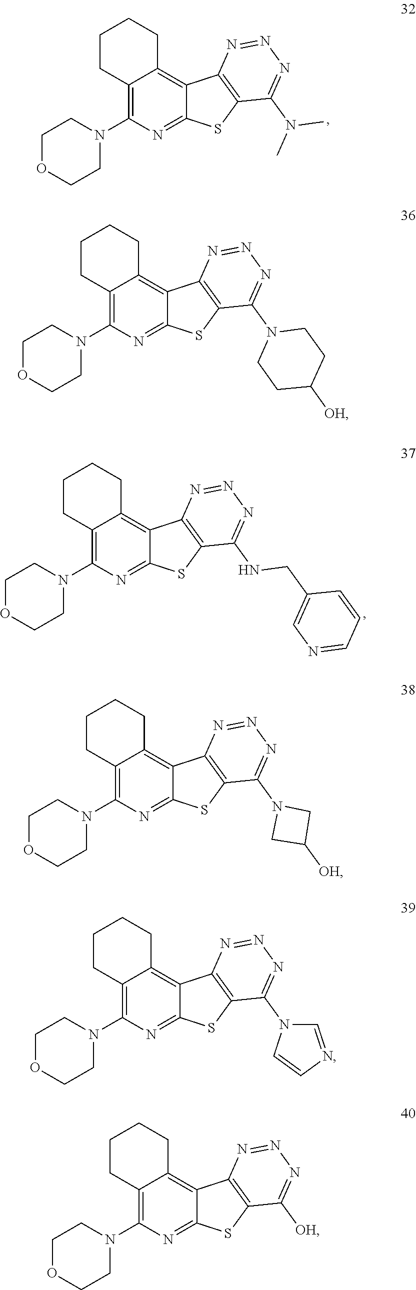 [SMILES: CN(C)c1nnnc2c1sc1nc(N3CCOCC3)c3c(c12)CCCC3 ; OC1CCN(c2nnnc3c2sc2nc(N4CCOCC4)c4c(c23)CCCC4)CC1 ; c1cncc(CNc2nnnc3c2sc2nc(N4CCOCC4)c4c(c23)CCCC4)c1 ; OC1CN(c2nnnc3c2sc2nc(N4CCOCC4)c4c(c23)CCCC4)C1 ; c1cn(-c2nnnc3c2sc2nc(N4CCOCC4)c4c(c23)CCCC4)cn1 ; Oc1nnnc2c1sc1nc(N3CCOCC3)c3c(c12)CCCC3]

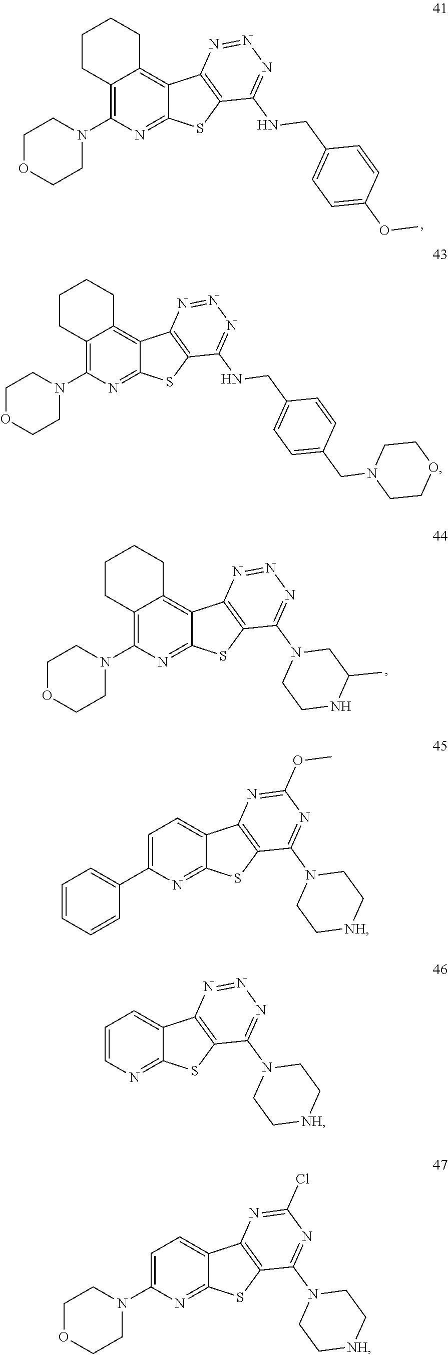 [SMILES: COc1ccc(CNc2nnnc3c2sc2nc(N4CCOCC4)c4c(c23)CCCC4)cc1 ; c1cc(CN2CCOCC2)ccc1CNc1nnnc2c1sc1nc(N3CCOCC3)c3c(c12)CCCC3 ; CC1CN(c2nnnc3c2sc2nc(N4CCOCC4)c4c(c23)CCCC4)CCN1 ; COc1nc(N2CCNCC2)c2sc3nc(-c4ccccc4)ccc3c2n1 ; c1cnc2sc3c(N4CCNCC4)nnnc3c2c1 ; Clc1nc(N2CCNCC2)c2sc3nc(N4CCOCC4)ccc3c2n1]

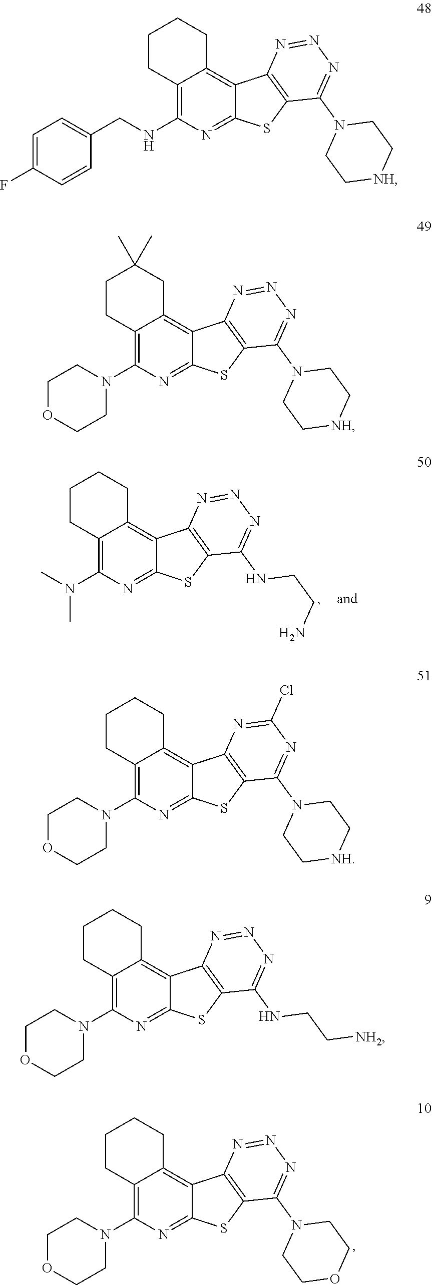

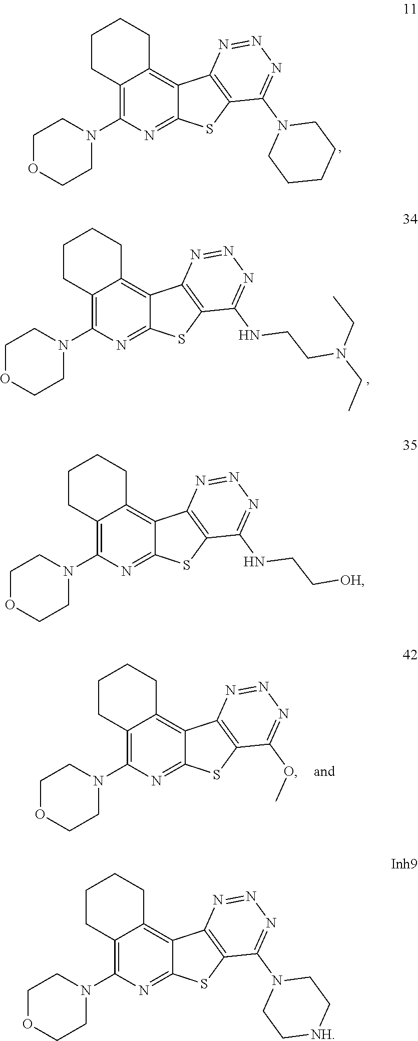 [SMILES: C1CCN(c2nnnc3c2sc2nc(N4CCOCC4)c4c(c23)CCCC4)CC1 ; CCN(CC)CCNc1nnnc2c1sc1nc(N3CCOCC3)c3c(c12)CCCC3 ; OCCNc1nnnc2c1sc1nc(N3CCOCC3)c3c(c12)CCCC3 ; COc1nnnc2c1sc1nc(N3CCOCC3)c3c(c12)CCCC3 ; C1CCc2c(c(N3CCOCC3)nc3sc4c(N5CCNCC5)nnnc4c23)C1]

In embodiments, the methods described herein are directed to treating a condition, wherein the condition is chemotherapy induced loss of body weight.

As in any embodiment above, the method wherein the loss of body weight is reduced from about 5% to about 30%.

As in any embodiment above, the method wherein the loss of body weight is reduced from about 10% to about 20%.

As in any embodiment above, the method wherein the loss of body weight is delayed by about 10% to about 30%.

As in any embodiment above, the method wherein the chemotherapy is a camptothecin derived antineoplastic agent.

As in any embodiment above, the method wherein the camptothecin derived antineoplastic agent is selected from the group consisting of camptothecin, diflomotecan, exatecan, gimatecan, irinotecan, karenitecin, lurtotecan, rubitecan, silatecan and topotecan.

As in any embodiment above, the method wherein the camptothecin derived antineoplastic agent is irinotecan.

As in any embodiment above, the method for treating a condition, wherein the condition is cancer.

As in any embodiment above, the method wherein the cancer is selected from the group consisting of melanoma, squamous cell cancer, lung cancer, cancer of the peritoneum, hepatocellular cancer, gastric or stomach cancer, pancreatic cancer, glioblastoma, glioblastoma multiforme, KRAS mutant solid tumors, indolent non-Hodgkin's lymphoma, chronic lymphocytic leukemia (CLL), diffuse large B-cell lymphoma, thyroid cancer, non-Hodgkin's lymphoma, basal cell carcinoma, hematological tumors, B-cell non-Hodgkin's lymphoma, acute myeloid leukemia (AML), cervical cancer, ovarian cancer, liver cancer, bladder cancer, hepatoma, breast cancer, colon cancer, rectal cancer, colorectal cancer, endometrial carcinoma, salivary gland carcinoma, kidney cancer, prostate cancer, vulval cancer, thyroid cancer, hepatic carcinoma, anal carcinoma, penile carcinoma, and head and neck cancer.

As in any embodiment above, the method wherein the cancer is breast cancer.

As in any embodiment above, the method wherein the treating cancer comprises slowing tumor growth.

As in any embodiment above, the method wherein the tumor growth is slowed by about 1% to about 30%.

As in any embodiment above, the method wherein the tumor growth is slowed by about 10% to about 25%.

As in any embodiment above, the method for treating a condition, wherein the condition is the presence of gut proteobacteria.

As in any embodiment above, the method wherein the amount of the gut proteobacteria is reduced by about 10% to about 50% when compared to the amount present prior to administering a compound of Formula I.

As in any embodiment above, the method wherein the amount of the gut proteobacteria is reduced by about 15% to about 40% when compared to the amount present prior to administering a compound of Formula I.

As in any embodiment above, the method wherein the administering a compound of Formula I modulates the ratio of gut proteobacteria to the remainder of gut bacteria to at least 1:5.

As in any embodiment above, the method wherein the administering a compound of Formula I modulates the ratio of gut proteobacteria to the remainder of gut bacteria to at least 1:20.

As in any embodiment above, the method wherein the administering a compound of Formula I modulates the ratio of gut proteobacteria to the remainder of gut bacteria to at least 1:50.

As in any embodiment above, the method wherein the condition is gastrointestinal distress caused by the treatment of a disease with an antineoplastic agent.

As in any embodiment above, the method wherein the antineoplastic agent is a camptothecin derived antineoplastic agent.

As in any embodiment above, the method wherein the camptothecin derived antineoplastic agent is selected from the group consisting of camptothecin, diflomotecan, exatecan, gimatecan, irinotecan, karenitecin, lurtotecan, rubitecan, silatecan and topotecan.

As in any embodiment above, the method wherein the camptothecin derived antineoplastic agent is irinotecan.

As in any embodiment above, the method wherein the compounds of Formula I improve the treatment when compared to treatment with Inh-1. In a further embodiment, the method wherein the efficacy of irinotecan is improved.

As in any embodiment above, the method further comprising administering an antineoplastic agent.

As in any embodiment above, the method wherein the administering comprises administering multiple doses or a single dose of a compound of Formula I.

As in any embodiment above, the method wherein the administering comprises administration of a single dose of a compound of Formula I.

As in any embodiment above, the method wherein the antineoplastic agent is irinotecan.

As in any embodiment above, the method wherein the method comprises administering to a subject prior to, concurrently with, or after administration of the antineoplastic agent an effective amount of a compound of Formula I.

As in any embodiment above, the method wherein the method comprises administering to a subject concurrently with the antineoplastic agent an effective amount of a compound of Formula I.

As in any embodiment above, the method wherein the antineoplastic agent may be administered in a greater number of doses when compared to administering the antineoplastic agent without administering a compound of Formula I.

As in any embodiment above, the method wherein the greater number of doses is in a range from about 50% to about 75% greater number of doses when compared to administering the antineoplastic agent without administering a compound of Formula I. In embodiments, the greater number of doses is in a range from about 5% to about 25%, from about 25% to about 50%, from about 75% to about 100%, from about 100% to about 150%. In embodiments, the greater number of doses is at least 5%, 10%, 25%, 50%, 75%, 100%, or 150% greater.

As in any embodiment above, the method wherein the greater number of doses is in a range from about 5 to about 10 more doses of antineoplastic agent. In embodiments, the number of doses is increased in the range from about 1 to about 5, from 5 to about 10, from 10 to about 15, or from about 15 to about 20 more doses. In embodiments, the number of doses is increased by at least 1, 2, 3, 4, 5, 6, 7, 8, 9, 10, 11, 12, 13, 14, 15, 16, 17, 18, 19, or 20 doses.

As in any embodiment above, the method wherein the antineoplastic agent may be administered for a greater period of time without serious side effects when compared to administering the antineoplastic agent without administering a compound of Formula I.

As in any embodiment above, the method wherein the greater period of time is in a range from about 50% to about 75% greater period of time. In embodiments, the greater period of time is in a range from about 5% to about 25%, from about 25% to about 50%, or from about 75% to about 100%. In embodiments, the greater period of time is at least 5%, 10%, 25%, 50%, 75%, or 100%.

As in any embodiment above, the method wherein the greater period of time is in a range from about 5 to about 10 more days. In embodiments, the number of days is increased in the range from about 1 to about 5, from 5 to about 10, from 10 to about 15, or from about 15 to about 20 more days. In embodiments, the number of days is increased by at least 1, 2, 3, 4, 5, 6, 7, 8, 9, 10, 11, 12, 13, 14, 15, 16, 17, 18, 19, or 20 days.

As in any embodiment above, the method wherein the method results in an increase in survival time when compared to administering the antineoplastic agent without administering a compound of Formula I.

As in any embodiment above, the method wherein the increase in survival time is in a range from about 25% to about 50% more time. In embodiments, the increase in survival time is in a range from about 1% to about 5%, from about 6% to about 10%, from about 11% to about 15%, from about 16% to about 20%, from about 21% to about 25%, from about 26% to about 30%, from about 31% to about 35%, from about 36% to about 40%, from about 41% to about 45%, about 46% to about 50%, about 51% to about 55%, from about 56% to about 60%, from about 61% to about 65%, from about 66% to about 70%, or from about 71% to about 75%. In an embodiment, the increase in survival time is an increase of at least 1%, 2%, 3%, 4%, 5%, 10%, 15%, 20%, 25%, 29% 30%, 35%, 40%, 43%, 45%, 50%, 55%, 60%, 65%, 70%, or 75%.

As in any embodiment above, the method wherein the compound of Formula I is administered at dose between about 0.001 μg/kg and about 1000 mg/kg.

In embodiments, the subject matter described herein is directed to a pharmaceutical composition comprising a compound of Formula I and a pharmaceutically acceptable carrier or excipient.

The subject matter described herein also includes pharmaceutically acceptable salts of compounds of Formula I.

If the compound of Formula I is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Illustrative examples of other suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The methods described herein provide methods for selectively inhibiting bacterial β-glucuronidases. In the methods, an effective amount of at least one selective β-glucuronidase inhibitor can be administered to a subject in need thereof. That is, a subject being treated with a chemotherapeutic agent, such as a camptothecin-derived antineoplastic agent or glucuronidase-substrate agents or compounds.

Also described herein are methods for improving efficacy of camptothecin-derived antineoplastic agents or glucuronidase-substrate agents or compounds by attenuating reactivation by bacterial β-glucuronidases of glucuronidated metabolites of camptothecin-derived antineoplastic agents or glucuronidase-substrate agents or compounds. In the methods, a therapeutically effective amount of at least one selective β-glucuronidase inhibitor can be administered to a subject having or about to have treatment with a chemotherapeutic agent, particularly a camptothecin-derived antineoplastic agent or any other glucuronidase-substrate agents or compounds.

Additionally, disclosed herein are wild-type, non-tumor bearing animal models showing that β-glucuronidase inhibitors significantly protect the GI from damage resulting from reactivation of administered irinotecan. Furthermore, the improvement in therapeutic efficacy of chemotherapeutic compounds upon concurrent β-glucuronidase inhibitor treatment is disclosed. A tumor xenograft model in athymic mice as well as a genetically engineered mouse model (GEMM) of breast cancer were employed.

The data described herein indicate that bacterial β-glucuronidase inhibition is an effective adjuvant therapy when administered in conjunction with compounds whose metabolites, when reactivated by the microbiota, inflict severe intestinal damage. Furthermore, the compounds are useful as monotherapy agents as described herein.

The compounds disclosed herein can be used in compositions and methods for inhibiting bacterial β-glucuronidases and for improving efficacy of camptothecin-derived antineoplastic agents or glucuronidase-substrate agents or compounds by attenuating the gastrointestinal distress caused by reactivation of glucuronidated metabolites of such agents. The presently disclosed compounds find use in selectively inhibiting the activity of one or more microbial beta-glucuronidase (GUS) enzymes found in the mammalian GI tract. The presently disclosed compounds reduce, inhibit, or otherwise diminish the activity of one or more GUS enzymes. The presently disclosed compounds may or may not be a specific GUS inhibitor. However, the GUS inhibitor is selective for microbial or bacterial beta-glucuronidase as compared to other non-microbial beta-glucuronidases. The methods comprise contacting GUS with an effective amount of a compound of Formula I.

The compounds and compositions can be used in methods for treating cancer to reduce the side effects of antineoplastic agents, such as camptothecin-derived antineoplastic agents. Thus, the gastrointestinal distress that typically accompanies treatment with an antineoplastic agent can be attenuated. The methods are also useful for attenuating or improving any adverse reactions associated with administration of glucuronidase-substrate agent(s) or compound(s). Methods of the present invention include administering to a subject in need thereof a therapeutically effective amount of at least one β-glucuronidase inhibiting agent that selectively inhibits bacterial β-glucuronidases from hydrolyzing glucuronides.

A method of ameliorating a side effect in a human in need thereof, comprising administering to a human an effective amount of a compound of Formula I.

A method of inhibiting a GUS comprising, contacting a GUS with a compound of Formula I.

By “contact” is intended bringing the compound within close enough proximity to the target GUS such that the compound is able to inhibit the activity of GUS. The compound can be contacted with GUS in vitro or in vivo via administration of the compound to a subject.

In embodiments, the methods are directed to:

A method for selectively inhibiting bacterial β-glucuronidases, the method comprising administering to a subject in need thereof an effective amount of at least one compound of Formula I. In embodiments, the bacteria are selected from the group consisting of a Bacteroides sp., Bifidobacterium sp., Catenabacterium sp., Clostridium sp., Corynebacterium sp., Enterococcus faecalis, Enterobacteriaceae, Lactobacillus sp., Peptostreptococcus sp., Propionibacterium sp., Proteus sp., Mycobacterium sp., Pseudomonas sp., Staphylococcus sp. and Streptococcus sp.

A method for improving camptothecin-derived antineoplastic agent efficiency, the method comprising administering to a subject prior to, concurrently with or after administration of a camptothecin-derived antineoplastic agent a therapeutically effective amount of at least one compound of Formula I.

A method for attenuating side effects in a subject being administered a camptothecin-derived antineoplastic agent, the method comprising administering prior to, concurrently with or after administration of a camptothecin-derived antineoplastic agent a therapeutically effective amount of at least one compound of Formula I.

In embodiments, the camptothecin-derived antineoplastic agent is selected from the group consisting of camptothecin, diflomotecan, exatecan, gimatecan, irinotecan, karenitecin, lurtotecan, rubitecan, silatecan and topotecan. In particular, the camptothecin-derived antineoplastic agent is irinotecan.

A method to alleviate gastrointestinal distress associated with chemotherapy comprising: a) administering to an animal an anti-cancer effective amount of a chemotherapeutic agent, and b) administering to the same animal an inhibitory effective amount of a one compound of Formula I. In an aspect of this embodiment, the chemotherapeutic active agent is a camptothecin-derived antineoplastic agent.

A method for improving the efficiency of a glucuronidase-substrate agent or compound, the method comprising administering to a subject prior to, concurrently with or after administration of an agent or compound a therapeutically effective amount of at least one compound of Formula I.

Methods for assessing β-glucuronidase activity are known in the art. See, e.g., Farnleitner et al. (2002) Water Res. 36:975-981; Fior et al. (2009) Plant Sci. 176:130-135; and Szasz (1967) Clin. Chem. 13:752-759. One of skill in the art is familiar with assays for testing the ability of an active compound for selectively inhibiting β-glucuronidases with minor or no toxicity to the bacteria that inhabit the gastrointestinal tract. β-glucuronidase activity of bacteria provided the selective β-glucuronidase inhibitor can be reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% when compared to bacteria not provided the selective β-glucuronidase inhibitor. In an embodiment, the reduction may be in a range of about 1-20%, about 21-40%, about 41-60%, about 61 to 80%, about 81-95%, or about 96-100%.

In one aspect, provided herein is a method for ameliorating the side effects, in particular the GI side effects, in the treatment of cancer in a subject in need thereof. The term “cancer” refers to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include melanoma, squamous cell cancer (e.g., epithelial squamous cell cancer), lung cancer including melanoma, multiple myeloma, small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, glioblastoma multiforme, KRAS mutant solid tumors, indolent non-Hodgkin's lymphoma, chronic lymphocytic leukemia (CLL), diffuse large B-cell lymphoma, thyroid cancer, non-Hodgkin's lymphoma, basal cell carcinoma, hematological tumors, B-cell non-Hodgkin's lymphoma, acute myeloid leukemia (AML), cervical cancer, ovarian cancer, liver cancer, bladder cancer, hepatoma, breast cancer, including triple negative breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Also included are “hematological malignancies” or “hematological cancer,” which are the types of cancer that affect blood, bone marrow, and lymph nodes. Hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin. Leukemias include Acute lymphoblastic leukemia (ALL), Acute myelogenous leukemia (AML), Chronic lymphocytic leukemia (CLL), Chronic myelogenous leukemia (CML), Acute monocytic leukemia (AMOL) and small lymphocytic lymphoma (SLL). Lymphomas include Hodgkin's lymphomas (all four subtypes) and Non-Hodgkin's lymphomas (NHL, all subtypes).

The presently disclosed compounds may be administered in any suitable manner known in the art. In some embodiments, the compound of Formula I, or a pharmaceutically acceptable salt thereof, is administered intravenously, intramuscularly, subcutaneously, topically, orally, transdermally, intraperitoneally, intraorbitally, by implantation, by inhalation, intrathecally, intraventricularly, intratumorally, or intranasally.

In some embodiments, the compound of Formula I is administered continuously. In other embodiments, the compound of Formula I is administered intermittently. Moreover, treatment of a subject with an effective amount of a compound of Formula I can include a single treatment or can include a series of treatments.

It is understood that appropriate doses of the active compound depends upon a number of factors within the knowledge of the ordinarily skilled physician or veterinarian. The dose(s) of the active compound will vary, for example, depending upon the age, body weight, general health, gender, and diet of the subject, the time of administration, the route of administration, the rate of excretion, and any drug combination.

It will also be appreciated that the effective dosage of a compound of Formula I or a pharmaceutically acceptable salt used for treatment may increase or decrease over the course of a particular treatment. Changes in dosage may result and become apparent from the results of diagnostic assays.

In some embodiments, a compound of Formula I is administered to the subject at a dose of between about 0.001 μg/kg and about 1000 mg/kg, between about 0.001 μg/kg and about 0.01 μg/kg, between about 0.01 μg/kg and about 0.1 μg/kg, between about 0.1 μg/kg and about 1 μg/kg, between about 1 μg/kg and about 10 μg/kg, between about 10 μg/kg and about 100 μg/kg, between about 100 μg/kg and about 1000 μg/kg (i.e., 1 mg/kg), between about 1 mg/kg and about 10 mg/kg, between about 10 mg/kg and about 100 mg/kg, between about 100 mg/kg and about 1000 mg/kg; including but not limited to about 0.001 μg/kg, 0.01 μg/kg, 0.05 μg/kg, 0.1 μg/kg, 0.5 μg/kg, 1 μg/kg, 10 μg/kg, 25 μg/kg, 50 μg/kg, 100 μg/kg, 250 μg/kg, 500 μg/kg, 1 mg/kg, 5 mg/kg, 10 mg/kg, 25 mg/kg, 50 mg/kg, 100 mg/kg, and 200 mg/kg.

In some embodiments, the subject that is administered a compound of Formula I is a mammal, such as domesticated animals (e.g., cows, sheep, cats, dogs, and horses), primates (e.g., humans and non-human primates such as monkeys), rabbits, and rodents (e.g., mice and rats). In some embodiments, the subject treated is a human.

In some embodiments, the methods for ameliorating the side effects or improving the efficacy of a chemotherapeutic drug comprising administering to the subject an effective amount of a compound of Formula I, or a pharmaceutically acceptable salt thereof, further comprises administering the chemotherapeutic drug. For example, a compound of Formula I and a chemotherapeutic agent may be administered sequentially (at different times) or concurrently (at the same time). A compound of Formula I and chemotherapeutic agent may be administered by the same route of administration or by different routes of administration.

The chemotherapeutic agent can be a camptothecin-derived antineoplastic agent. As used herein, “a camptothecin-derived antineoplastic agent” and the like means a cytotoxic quinoline alkaloid that inhibits the DNA enzyme topoisomerase I. Camptothecin-derived antineoplastic agents include, but are not limited to, camptothecin (i.e., (S)-4-ethyl-4-hydroxy-1H-pyrano[3′,4′:6,7]indolizino[1,2-b]quinoline-3,14-(4H,12H)-dione); diflomotecan (i.e., (5R)-5-ethyl-9,10-difluoro-1,4,5,13-tetrahydro-5-hydroxy-3H,15H-oxepino[3′,4′:6,7]indolizino[1,2-b]quinoline-3,15-dione); exatecan (i.e., (1S,9S)-1-amino-9-ethyl-5-fluoro-1,2,3,9,12,15-hexahydro-9-hydroxy-4-methyl-10H,13H-benzo(de)pyrano(3′,4′:6,7)indolizino(1,2-b)quinoline-10,13-dione); gimatecan (i.e., (4S)-11-((E)-((1,1-dimethylethoxy)imino)methyl)-4-ethyl-4-hydroxy-1,12-dihydro-14H-pyrano(3′,4′:6,7)indolizino(1,2-b)quinoline-3,14(4H)-dione); irinotecan (i.e., (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate); karenitecin (i.e., (4S)-4-ethyl-4-hydroxy-11-(2-trimethylsilyl)ethyl)-1H-pyrano[3′,4′:6,7]indolizino[1,2-b]quinoline-3,14(4H,12H)-dione); lurtotecan (i.e., 7-(4-methylpiperazinomethylene)-10,11-ethylenedioxy-20(S)-camptothecin); rubitecan (i.e., (4S)-4-ethyl-4-hydroxy-10-nitro-1H-pyrano[3′,4′:6,7]indolizino[1,2-b]quinoline-3,14(4H,12H)-dione); silatecan (i.e., 7-tert-butyldimethylsilyl-10-hydroxycamptothecin); and topotecan (i.e., (S)-10-[(dimethylamino)methyl]-4-ethyl-4,9-dihydroxy-1H-pyrano[3′,4′:6,7]indolizino[1,2-b]quinoline-3,14(4H,12H)-dione).

Of particular interest is irinotecan (CPT-11 or Camptosar®), which is a potent camptothecin-derived antineoplastic agent for treating solid malignancies of the brain, colon and lung, as well as refractory forms of leukemia and lymphoma. Irinotecan is a prodrug that must be converted into its active form, SN-38 (7-ethyl-10-hydroxy-camptothecin), to have antineoplastic activity. During its excretion, SN-38 is glucuronidated to SN-38 glucuronide (SN-38G) by phase II drug metabolizing UDP-glucuronosyltransferases.

The term “glucuronidase-substrate agent(s) or compound(s)” refers to any drug, agent or compound or, in particular, a metabolite thereof that can be a substrate for glucuronidase. Thus, in some instances, a drug, compound or agent that is not itself a substrate, but is metabolized to a substrate is encompassed by the term as used herein. Any drug, compound or agent or metabolite thereof that is glucuronidated, also referred to as glucuronides, can be a substrate for glucuronidase and is also described herein as glucuronidase-substrate agent(s) or compound(s). Many drugs, agents or compounds undergo glucuronidation at some point in their metabolism. Alternatively, the drug, agent or compound may be a glucuronide pro-drug. These glucuronides may have different properties than the parent drug, agent or compound. Glucuronidation can modulate the potency of some drugs: the 6-glucuronide of morphine is a more potent analgesic than the parent compound, whereas the 3-glucuronide is a morphine antagonist. In addition, steroid glucuronidation can produce more active or toxic metabolites under pathophysiological conditions or during steroid therapies.

Drugs, agents or compounds or metabolites thereof which are substrates for glucuronidase can have their respective properties altered by glucuronidase hydrolysis. In a specific, non-limiting example, if the drug, agent, compound or metabolite thereof has been metabolized to a glucuronide, the hydrolysis of the glucuronide can reactivate the drug, agent, compound or metabolite thereof. In many cases, this reactivation can cause adverse reactions. For example, if a glucuronide drug, agent or compound or metabolite thereof is present in the gut, glucuronidase hydrolysis in the gut can lead to gastrointestinal distress.

The methods described herein are useful for attenuating, ameliorating or improving the adverse reactions, such as gastrointestinal distress, caused by the action of glucuronidase on a drug, agent or compound or, in particular, a metabolite thereof. As described fully elsewhere herein, hydrolysis of glucuronides can lead to adverse reactions. The methods described herein inhibit or decrease the activity of β-glucuronidases. The methods can therefore be useful to attenuate, ameliorate or improve adverse reactions, such as gastrointestinal distress, associated with administering such drugs, agents or compounds. The methods can also improve the tolerance of any such drug, agent or compound or metabolite thereof that can form a glucuronide. As such, administration of a glucuronidase inhibitor can rescue or improve a treatment with any drug, agent or compound, wherein glucuronidase hydrolysis of a glucuronide related to the drug, agent, compound or metabolite thereof is causing one or more adverse reactions, particularly gastrointestinal distress or toxicity. Patient compliance and outlook would also improve with the lessening of adverse reactions.

As mentioned above, the term “glucuronidase-substrate agent(s) or compound(s)” refers to any drug, agent or compound or, in particular, a metabolite thereof that can be a substrate for glucuronidase. These can include any chemical compound useful in the treatment of disease, for example, but not limited to, cancer. Examples of such chemotherapeutic agents include NSAIDs, sorafenib, alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, and floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an anti-estrogen such as fulvestrant; EGFR inhibitor such as erlotinib or cetuximab; an anti-VEGF inhibitor such as bevacizumab; arinotecan; rmRH (e.g., ABARELIX®); 17AAG (geldanamycin derivative that is a heat shock protein (Hsp) 90 poison), and pharmaceutically acceptable salts, acids or derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifene citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); and (x) pharmaceutically acceptable salts, acids and derivatives of any of the above.

As used herein, a “chemotherapeutic regimen” is a schedule or course for administering chemotherapy agents to a cancer patient. The methods described herein may enhance a chemotherapeutic regimen.

As used herein, the terms “enhance” or “enhancing” mean to intensify, increase, or improve the quality, value, or extent of, for example, the regimen or treatment being enhanced. Enhancing may lead to the avoidance of side-effects or therapeutic failure.

As used herein, the terms “increase,” “increases,” “increased,” “increasing”, or “improve”, and similar terms indicate an elevation in the specified parameter of at least about 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more.

As used herein, the phrases “modulates the ratio of gut proteobacteria” or “modulate the amount of gut proteobacteria” refers to an increase, decrease, or other alteration in the relative amounts of bacteria. A non-limiting example of modulating the amount of gut proteobacteria is to lessen the amount of gut proteobacteria in relation to the remainder of gut bacteria.

Additional methods show that the microbiome-targeted inhibitors reduce tumor size on their own without an antineoplastic compound, such as irinotecan. In an embodiment, the tumor size may be reduced in a range by about 1-99%, 2-98%, 3-97%, 4-96%, 5-95%, 10-90%, 20-80%, 30-70%, or 40-60%.

Additionally, the inhibitors described herein may reduce the amount of chemotherapy induced loss of body weight. As used herein, the phrase “chemotherapy induced loss of body weight” refers to the amount of body weight lost in a subject following treatment with a chemotherapeutic. Such body weight loss may be in a range of about 1-40%, 2-35%, 5-30%, or 10-20%.

Additional methods may delay the chemotherapy induced body weight loss. As used herein, a “delay” in the chemotherapy induced body weight loss refers to the amount of time that the weight loss is postponed. The delay in chemotherapy induced body weight loss body weight may be by about 1-50%, 5-40%, 10-30%, or 15-25%.

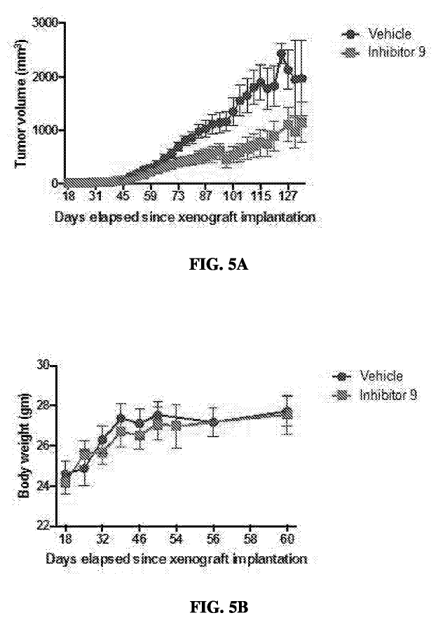

Furthermore, the inhibitors described herein may slow tumor growth, as seen, for example, in FIG. 5A. As used herein, the phrase “slow tumor growth” or “slowing tumor growth” refers to a reduction in the rate at which a tumor grows. Tumor growth rate may be reduced in a range of about 1-30%, 5-25%, or 10-20%.

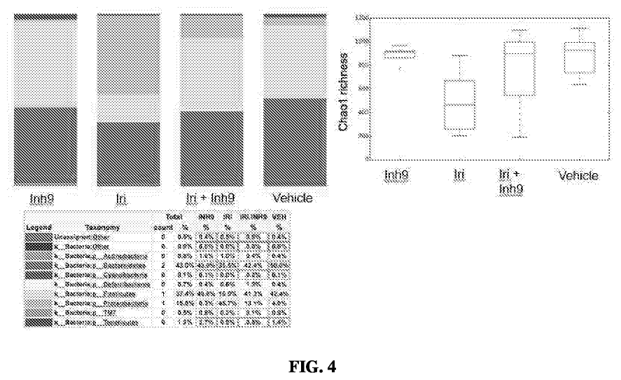

Still further methods disclose the consequences ofβ-glucuronidase inhibition on the composition of the GI microbiota in two preclinical models of human breast cancer. The impact of irinotecan with and without a β-glucuronidase inhibitor on the composition of the GI microbiota was examined using 16S rRNA sequencing, and revealed remarkable changes reflective of a specific mechanism of inhibitor action, as depicted, for example, in FIG. 4. Specifically, the inhibitors significantly change the composition of the bacteria in the intestinal tract. In an embodiment, the amount of gut proteobacteria present following administration of a compound of Formula I may be reduced when compared to the amount of gut proteobacteria present prior to administration of a compound of Formula I. The gut proteobacteria may be reduced in a range from about 1-99%, 2-98%, 3-97%, 4-96%, 5-95%, 10-90%, 20-80%, 30-70%, or 40-60%. Additionally, the ratio of gut proteobacteria to the remainder of the gut bacteria may be changed. Such a ratio may be, for gut proteobacteria to remainder of gut bacteria, in the range of 1:1, 1:2, 1:3, 1:4, 1:5, 1:10, 1:15, 1:20, 1:25, 1:30, 1:40, 1:50, 1:60, 1:70, 1:80, 1:90, 1:95, 1:96, 1:97, 1:98, or 1:99.

Thus, the inhibitors described herein have applications to treating cancer and controlling the types of bacteria in the intestines.

### III. Compositions

Compounds of Formula I can be formulated in accordance with standard pharmaceutical practice as a pharmaceutical composition. According to this aspect, there is provided a pharmaceutical composition comprising a compound of Formula I in association with a pharmaceutically acceptable excipient, such as a carrier or diluent.

A typical formulation is prepared by mixing a Formula I compound and one or more excipients. Suitable excipients are well known to those skilled in the art and include materials such as carbohydrates, waxes, water soluble and/or swellable polymers, hydrophilic or hydrophobic materials, gelatin, oils, solvents, water and the like. The particular excipients used will depend upon the means and purpose for which the compound of Formula I is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of Formula I or pharmaceutical composition thereof) or aid in the manufacturing of the pharmaceutical product (i.e., medicament).

The formulations may be prepared using conventional dissolution and mixing procedures. For example, the bulk drug substance (i.e., compound of Formula I or stabilized form of the Formula I compound) (e.g., complex with a cyclodextrin derivative or other known complexation agent) is dissolved in a suitable solvent in the presence of one or more of the excipients described above. The compound of Formula I is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. In one embodiment, the container is a blister pack.

Pharmaceutical formulations may be prepared for various routes and types of administration. For example, a compound of Formula I having the desired degree of purity may optionally be mixed with pharmaceutically acceptable excipients (Remington's Pharmaceutical Sciences (1980) 16^{th} edition, Osol, A. Ed., Mack Publishing Co., Easton, Pa.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compounds of Formula I can be sterile. In particular, formulations to be used for in vivo administration must be sterile. Such sterilization is readily accomplished by filtration through sterile filtration membranes.

The compound ordinarily can be stored as a solid composition, a lyophilized formulation or as an aqueous solution.

The pharmaceutical compositions comprising a compound of Formula I can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations. For example, dosing may be in a single dose or multiple doses. As used herein, “comprising a single dose’ is a single dose while other steps may be performed.

Acceptable excipients are nontoxic to recipients at the dosages and concentrations employed, and may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polysorbates (e.g., TWEEN™), poloxamers (e.g., PLURONICS™) or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences.

Sustained-release preparations of Formula I compounds may be prepared. Suitable examples of sustained-release preparations include semipermeable matrices of solid hydrophobic polymers containing a compound of Formula I, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as injectable microspheres composed of lactic acid-glycolic acid copolymer and leuprolide acetate (LUPRON DEPOT™) and poly-D-(−)-3-hydroxybutyric acid.

The formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound of Formula I suitable for oral administration may be prepared as discrete units such as pills, capsules, cachets or tablets each containing a predetermined amount of a compound of Formula I.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs may be prepared for oral use. Formulations of compounds of Formula I intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Aqueous suspensions of Formula I compounds contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of Formula I may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such 1,3-butanediol. The sterile injectable preparation may also be prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The subject matter further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

In particular embodiments the pharmaceutical composition comprising the presently disclosed compounds further comprise a chemotherapeutic agent.

### IV. Experimental and Discussion

Irinotecan is widely-used to treat a range of solid tumors, but its efficacy is severely limited by gastrointestinal (GI) tract damage and delayed diarrhea. GI toxicities are caused by gut microbial beta-glucuronidase (GUS) enzymes. Targeted bacterial GUS inhibitors have been shown in mice to alleviate irinotecan-induced GI tract damage and resultant diarrhea. Critical questions remain, however, including how quickly irinotecan-induced damage appears, how GUS inhibition affects irinotecan efficacy, and how irinotecan and GUS inhibitor impact gut microbial composition. Addressed herein are these questions using in vitro and ex vivo biochemistry, structural biology, mouse tumor models and microbiome sequencing. Irinotecan causes apoptotic crypt damage in the murine small intestine within 24 hours, several days before colonic injury and diarrhea are observed. In a triple-negative breast cancer xenograft model, GUS inhibition prevents intestinal toxicity but maintains the anti-tumor efficacy of irinotecan. However, in a genetically-engineered mouse model (GEMM) of breast cancer, GUS inhibition both alleviates gut damage and dramatically enhances irinotecan's ability to shrink tumors. Irinotecan induces a marked shift in the composition of the gut microbiota in the athymic xenograft mice, including a ten-fold increase in the levels of Proteobacteria; in these immune-compromised mice, these changes are effectively blunted by GUS inhibition. In immune-competent GEMM animals, however, irinotecan causes a smaller increase in Proteobacteria and GUS inhibition has no effect microbial composition. Thus, disclosed herein is that GUS inhibition is capable of dramatically enhancing the anti-tumor efficacy of irinotecan in a cancer GEMM while not altering the composition of the gut microbiota.

Irinotecan (7-ethyl-10-[4-(1-piperidino]-carbonyloxy-camptothecin, also known as CPT-11) is a first-line chemotherapeutic for colorectal and pancreatic cancers, and is frequently administered as a cocktail with 5-fluorouracil and/or oxaliplatin. Ongoing clinical trials seek to determine whether irinotecan use may be efficacious for tumors of the breast and ovaries, as well as leukemias (ClinicalTrials.gov ID NCT00005626), sarcomas, (NCT00509860), etc. Therapeutically-relevant doses of irinotecan cause both myelosuppression and gastrointestinal (GI) side effects, including mucositis and late-onset diarrhea that are often severe enough to reduce or halt therapy. In terms of GI toxicity, 88% of patients develop diarrhea of all grades, and 30% suffer experience life-threatening grade 3-4 diarrhea (1-3). Although anti-motility drugs like loperamide can limit some gut toxicity, irinotecan-induced diarrhea is often refractory and is an unmet clinical need.

The irinotecan prodrug is converted by non-specific esterases into SN-38, a potent topoisomerase I inhibitor. Topoisomerase I is critical for resolving DNA superhelical tension during DNA replication, and is highly expressed in rapidly proliferating cells such as those in tumors and the intestinal epithelium, which self-renews approximately once every five days. SN-38 is detoxified through addition of glucuronic acid (GA) by UDP-glucuronosyltransferases to form SN-38-G, a compound that is marked for elimination via the gastrointestinal (GI) tract. The gut microbiota, however, express β-glucuronidase (GUS) enzymes, glycosyl hydrolases that remove GA from a range of endogenous and xenobiotic glucuronides, including SN-38-G. Bacteria use the Entner-Doudoroff pathway to convert GA into pyruvate, which then enters the TCA cycle to form ATP; thus, for the microbiota, GA serves as an energetically-useful source of carbon (4).

Reactivation of SN-38 in the gut damages the intestinal epithelium, causing bleeding diarrhea and acute weight loss in animal models (5). In humans, broad-spectrum antibiotics diminish the GI side effects of irinotecan, a result that implicated the intestinal microbiota in inflicting gut toxicity. Studies in germ-free rats further supported a role for the microbiota in mediating the GI side effects of irinotecan (5, 6). Moreover, irinotecan was shown to alter the composition of the gut microbiota in rats GI system (7-9). Depleting the microbiota with antibiotics has deleterious consequences including impaired immune function and reduced metabolic capacity (10). Furthermore, maintenance of microbial diversity has beneficial effects, including reduced incidence of colorectal tumors in a genotoxin model (11, 12); enhanced ability to fend off influenza virus (11), improved insulin sensitivity (13), and reduced incidence of asthma (14). Thus, a need is to to alleviate irinotecan-induced gut toxicity without the use of antibiotics.

As disclosed herein, irinotecan toxicity could be alleviated with non-lethal compounds that inhibit gut microbial GUS enzymes without affecting the mammalian GUS ortholog. Human GUS is essential, as mutations in this gene cause the lethal lysosomal disease Sly Syndrome (REF). Chemotypes were identified with high-throughput screening that serve as potent microbial GUS inhibitors, are selective for bacterial GUS enzyme, and are non-lethal to cultured microbial and mammalian cells. Naïve Balb/c mice treated with therapeutically-relevant doses of irinotecan exhibited lethal intestinal toxicity within 10 days, while co-treatment with an initial GUS-targeted compound, Inhibitor 1, alleviated gut toxicity and diarrhea. A second chemotype, Inhibitor 5, achieved similar effects in the same mouse model (Roberts, et al, Redinbo, Mol. Pharm. 2013). Kong et al (15) have replicated our findings for both Inhibitor 1 and using amoxapine, an FDA-approved tricyclic antidepressant used for major depressive disorder and has properties resembling atypical antipsychotics. However, recently, Yang (16, 17) demonstrated that amoxapine incompletely blocks the GUS activity in human fecal extracts. Furthermore, amoxapine reduced the viability of bacteria cultured in vitro, further underscoring the need for potent GUS inhibitors that maintain microbial diversity within the gut. Beyond irinotecan, the intestinal toxicity of NSAIDs, which are also glucuronidated and reactivated by GUS enzymes in the gut, have also been alleviated using targeted microbial GUS inhibitors (LoGuidice et al, 2012 PMID:22328575; Saitta et al, 2014 PMID 23829165; Mani et al, 2014 PMID 24160697; Boelsterli et al, 2013 PMID 23091168) and mechanisms of intestinal NSAID toxicity has been clarified (Bhatt et al, 2017). Furthermore, Inhibitor 1 has been shown in rats to reduce NSAID-induced intestinal anastomosis after surgical resection (Yauw et al., 2018). As such, inhibiting gut microbial GUS enzymes offers potential health benefits beyond alleviating chemotherapy-induced diarrhea.

As disclosed herein is an understanding of structural features critical for specificity and potency of microbial β-glucuronidase inhibitors (18-20). Also disclosed is that GUS does not change the plasma concentrations of irinotecan or its metabolites (20). Further shown is a collection of the first atlas of β-glucuronidase enzymes found in the human gut microbiome, showing that the 279 unique enzymes identified from extant Human Microbiome Project data can be sorted into six structural categories based on active site features (16). Representative enzymes from each category, which span the dominant gut microbial phyla, were cloned and purified and the resulting data showed that structurally-distinct GUS enzymes process chemically-distinct GUS substrates (16). Also disclosed herein is an examination of what GUS enzymes efficiently process SN-38-G and how these enzymes are inhibited by Inhibitor 1 and a different chemotype, represented by Inh9.

Also explored was the potential for significantly improving the efficacy of irinotecan in mouse models of cancer. The models used are breast cancer models, in particular the highly aggressive, hormone-insensitive triple negative breast cancer (TNBC). Although irinotecan is currently used for pancreatic and colorectal cancer, breast cancer models were chosen for several reasons. First, irinotecan is currently in clinical trials to treat metastatic breast cancers (ClinicalTrials.gov ID NCT00003351). Second, breast tumors are palpable through the course of the animal study, providing information on tumor growth without requiring animal sacrifice. Third, it has been established that the tumor microenvironment in the intestine significantly reshapes the microbiota relative to the healthy GI tract (reviewed in (21). The inflammatory environment facilitates the growth of facultative anaerobes in the tumor-ridden colon, in contrast to allowing the persistence of strict anaerobes in the healthy colon (REFs). Therefore, it was tested whether GUS inhibitor co-treatment would alter the efficacy of irinotecan treatment in mouse models of human TNBC.

Several issues were explored related to the in vivo use of microbiome-targeted therapeutics to address irinotecan efficacy and toxicity. First, it was examined how quickly irinotecan toxicity appears in the murine GI tract, and it was established that GUS-targeted inhibitors alleviate even early gut epithelial damage. Second, the degree to which GUS-targeted inhibitors could enhance the antitumor effects of irinotecan was assessed in two mouse models of breast cancer. Third, it was determined how irinotecan and GUS-targeted inhibitors impact the composition of the gut microbiota in treated mice. As disclosed herein, it is established that GUS-targeted inhibitors can dramatically enhance irinotecan's efficacy without altering the composition of the gut microbiota, paving the way toward the translation of this approach to human studies.

A. Preparation of Formula I Compounds

Compounds of Formula I may be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g. Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Synthetic chemistry transformations and protecting group methodologies (protection and deprotection) useful in synthesizing Formula I compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds of Formula I may be prepared singly or as compound libraries comprising at least 2, for example 5 to 1,000 compounds, or 10 to 100 compounds. Libraries of compounds of Formula I may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or pharmaceutically acceptable salts thereof.

The General Procedures and Examples provide exemplary methods for preparing Formula I compounds. Those skilled in the art will appreciate that other synthetic routes may be used to synthesize the Formula I compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art.

In preparing compounds of Formulas I, protection of remote functionality (e.g., primary or secondary amine) of intermediates may be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz or CBZ) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

The following examples are offered by way of illustration and not by way of limitation.

### EXAMPLES

### Example 1

### Synthesis of Inh9 and Compounds 2-8

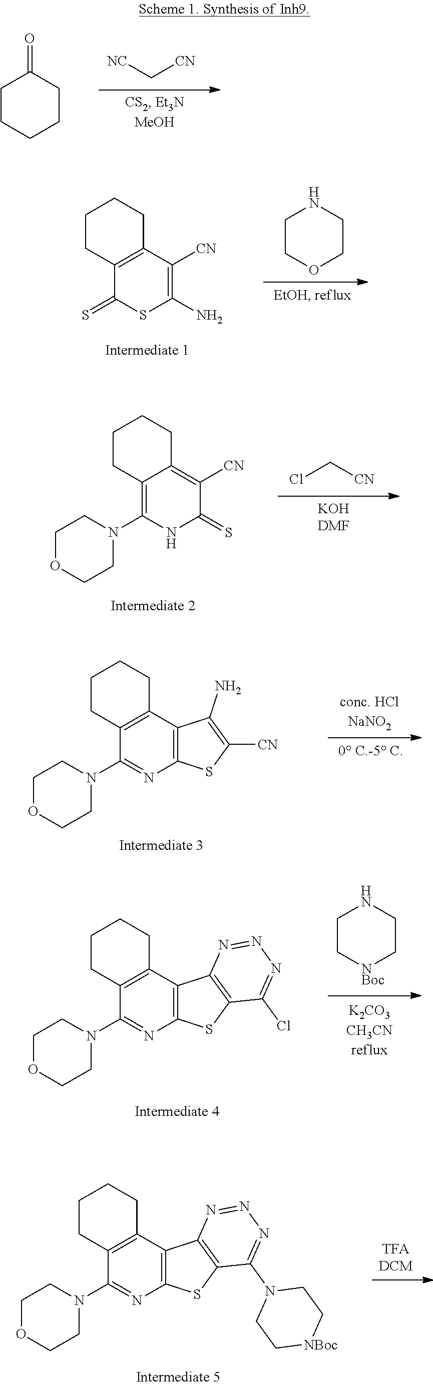

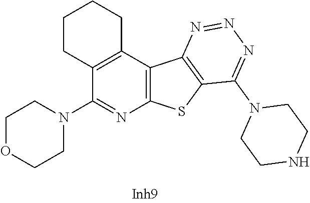 [SMILES: C1CCc2c(c(N3CCOCC3)nc3sc4c(N5CCNCC5)nnnc4c23)C1]

Compounds 2-8 in Table 1 were prepared by a similar method as Inh9 from intermediate 4.

### Synthesis of Intermediate 1

To a solution of cyclohexanone (5.0 g, 5.3 mL, 51 mmol, 1.0 eq.) in 16 mL of methanol was slowly added carbon disulfide (7.8 g, 6.1 mL, 102 mmol, 2.0 eq.) and malononitrile (3.4 g, 51 mmol, 1.0 eq.), and the mixture was stirred for 5 minutes while maintaining the temperature below 20° C. Triethylamine (2.5 mL) was added, and the reaction mixture was stirred overnight at rt. Precipitated product was filtered, washed with methanol and vacuum dried to provide intermediate 1 as an orange solid (4.3 g, 40%).

¹H NMR (400 MHz, DMSO-d₆) δ 8.69 (s, 2H), 2.68 (m, 2H), 2.59 (m, 2H), 1.68 (m, 4H)

LC-MS (λ=254 nm): 99%, t_{R}=5.9 min. MS (ESI+): 223 [M+H]⁺

### Synthesis of Intermediate 2

To a suspension of intermediate 1 (4.0 g, 18 mmol, 1.0 eq.) in ethanol (15 mL) was added morpholine (8 mL, 90 mmol, 5 eq.) and the mixture was heated under reflux overnight. Precipitated product was cooled to rt, degassed with nitrogen, filtered, washed with ethanol and vacuum dried to obtain intermediate 2 as an orange solid (3.8 g, 76%).

¹H NMR (400 MHz, DMSO-d₆) δ 4.33-4.27 (m, 1H), 3.74 (m, 4H), 3.67 (m, 4H), 3.55-3.50 (m, 1H), 2.61 (t, J=5.8 Hz, 2H), 2.41 (t, J=5.8 Hz, 2H), 1.77-1.65 (m, 2H), 1.62-1.52 (m, 2H)

LC-MS (λ=254 nm): 99%, t_{R}=4.9 min. MS (ESI+): 276 [M+H]⁺

### Synthesis of Intermediate 3

To a solution of intermediate 2 (3.0 g, 11 mmol, 1.0 eq.) in 22 mL of DMF was added 2-chloroacetonitrile (0.8 mL, 11.99 mmol, 1.1 eq.) and stirred at rt for an hour. Then the first portion of aq. KOH (10% w/v, 5.5 mL) was added to the reaction mixture and it was stirred at rt overnight, after which a second portion of aq. KOH (10% w/v, 5.5 mL) was added to the reaction mixture and stirred for another 4 hours at rt. Water (50 mL) was added to the precipitated solid product after which it was filtered and vacuum dried to obtain intermediate 3 as a pinkish yellow solid (2.36 g, 69%).

¹H NMR (400 MHz, DMSO-d₆) δ 6.35 (s, 2H), 3.84-3.70 (m, 4H), 3.27 (t, J=6.5 Hz, 2H), 3.19-3.10 (m, 4H), 2.64 (t, J=5.7 Hz, 2H), 1.88-1.75 (m, 2H), 1.70-1.58 (m, 2H)

LC-MS (λ=254 nm): 99%, t_{R}=5.9 min. MS (ESI+): 315 [M+H]⁺

### Synthesis of Intermediate 4

A solution of sodium nitrite (1.5 g, 22 mmol, 3.0 eq.) in water (7.3 mL) was added dropwise, over 30 minutes to a suspension of intermediate 3 (2.3 g, 7.3 mmol, 1.0 eq.) in conc. HCl acid (15 mL) at 0-5° C. The mixture was stirred for an hour at 0-5° C. and then allowed to stir at rt overnight. Water (100 mL) was added to the precipitated product after which it was filtered, washed with water, and vacuum dried to obtain intermediate 4 as a yellow solid (2.4 g, 92%).

¹H NMR (400 MHz, Chloroform-d) δ 3.93-3.85 (m, 2H), 3.75 (t, J=6.6 Hz, 1H), 3.48-3.41 (m, 2H), 2.74 (t, J=5.9 Hz, 1H), 2.08-1.97 (m, 1H), 1.88-1.77 (m, 1H)

LC-MS (λ=254 nm): 99%, t_{R}=6.4 min. MS (ESI+): 362 [M+H]⁺

### Synthesis of Intermediate 5

To a solution of intermediate 4 (85 mg, 0.23 mmol, 1.0 eq.) in CH₃CN (2 mL) was added K₂CO₃ (300 mg) and 1-Boc-piperazine (110 mg, 0.59 mmol, 2.5 eq.) and the reaction mixture was heated under reflux overnight. Upon completion, the reaction was quenched by addition of 20 mL of sat. NaHCO₃ solution. The aqueous phase was extracted with CH₂Cl₂ (3×20 mL) and the organic layers were combined, washed with brine, dried over anhydrous Na₂SO₄, and filtered. Solvent was removed by rotary evaporation to obtain a dark brown crude material. The crude material was adsorbed onto silica gel and purified by normal phase automated Teledyne Isco chromatography using a CH₂Cl₂/MeOH/NH₃ solvent system. Intermediate 5 was obtained as a pale yellow solid (50 mg, 43%).

¹H NMR (400 MHz, Chloroform-d) δ 4.08-4.02 (m, 4H), 3.92-3.83 (m, 4H), 3.77 (t, J=6.7 Hz, 2H), 3.68-3.60 (m, 4H), 3.38-3.30 (m, 4H), 2.73 (t, J=5.8 Hz, 2H), 2.03-1.94 (m, 2H), 1.85-1.76 (m, 2H), 1.49 (s, 9H)

LC-MS (λ=254 nm): 99%, t_{R}=6.5 min. MS (ESI+): 512 [M+H]⁺

### Synthesis of Inh9

To a solution of intermediate 5 (50 mg, 0.098 mmol) in CH₂Cl₂ (1 mL) at 0° C. was added trifluoroacetic acid (1 mL) and the reaction mixture was stirred until completion at room temperature. The solution was washed with saturated NaHCO₃ solution, dried over anhydrous Na₂SO₄, filtered, and vacuum concentrated to obtain Inh9 as a yellow solid (40 mg, 99%).

¹H NMR (400 MHz, Chloroform-d) δ 9.91 (s, 1H), 4.36 (s, 4H), 3.92-3.79 (m, 4H), 3.68 (t, J=6.2 Hz, 2H), 3.48 (s, 3H), 3.40-3.31 (m, 4H), 2.71 (t, J=5.6 Hz, 2H), 1.97 (m, 2H), 1.79 (m, 2H)

LC-MS (λ=254 nm): 99%, t_{R}=5.0 min. MS (ESI+): 412 [M+H]⁺

### Example 2

### Synthesis of Compounds 12-17

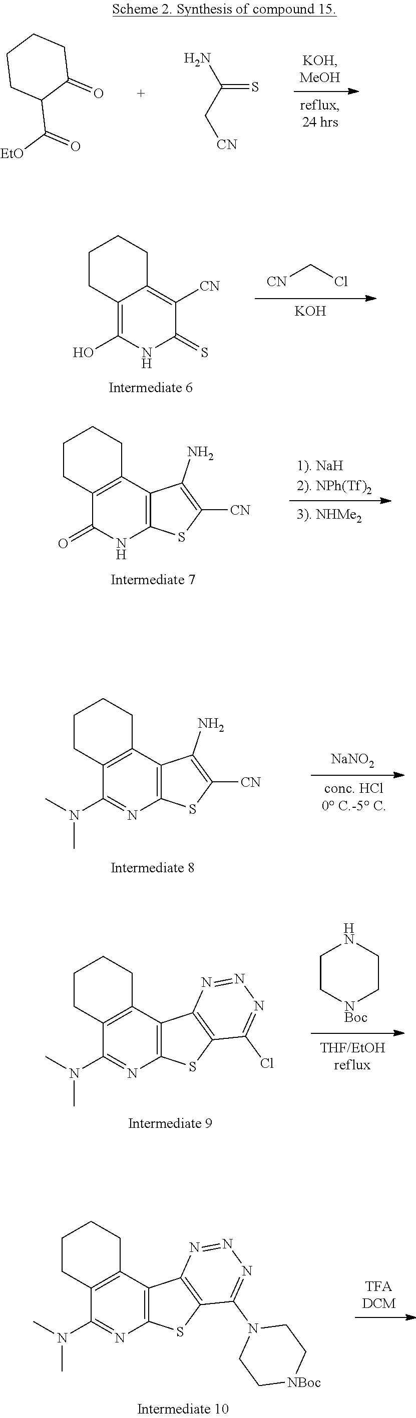

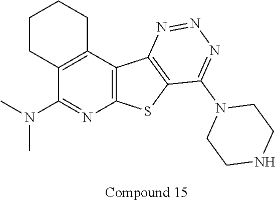 [SMILES: CN(C)c1nc2sc3c(N4CCNCC4)nnnc3c2c2c1CCCC2]

Intermediates 6 and 7 were synthesized according to the literature (J. C. A. Hunt et al. Bioorg. Med. Chem. Lett. 17 (2007) 5222-5226).

Compounds 12-14 and 16-17 in Table 1 were prepared by a similar method as compound 15 from intermediate 7.

### Synthesis of Intermediate 8

Intermediate 7 (500 mg, 2.04 mmol, 1.0 eq.) was added portion-wise to a stirred suspension of sodium hydride (60% dispersion in mineral oil, 85 mg, 2.12 mmol, 1.1 eq.) in DMF (10.3 mL) under nitrogen at 0° C. The reaction mixture was stirred at room temperature for 35-40 min. To the resultant solution was added N-phenyl-trifluoromethanesulfonimide (729 mg, 2.04 mmol, 1.0 eq.) in one portion. After 20 min., dimethylamine (1.06 mL, 2.12 mmol, 1.1 eq.) was added to the reaction mixture and stirred overnight. The reaction was slowly quenched with water (5 mL) and extracted with ethyl acetate (3×20 mL). The combined organic fractions were washed with saturated NaHCO₃ solution, dried over anhydrous Na₂SO₄, filtered, concentrated under reduced pressure, and purified by reverse phase automated Teledyne Isco chromatography using a MeOH/H₂O/0.1% acetic acid solvent system. Intermediate 8 was obtained as pale yellow solid (255 mg, 46%).

¹H NMR (400 MHz, DMSO-d₆) δ 3.22 (t, J=6.5 Hz, 2H), 2.85 (s, 6H), 2.59 (t, J=5.8 Hz, 2H), 1.84-1.72 (m, 2H), 1.60 (m, 2H)

LC-MS (λ=254 nm): 99%, t_{R}=6.1 min. MS (ESI+): 273 [M+H]⁺

### Synthesis of Intermediate 9

A solution of sodium nitrite (266 mg, 3.9 mmol, 3.0 eq.) in water (2 mL) was added dropwise over 30 minutes to a suspension of intermediate 8 (350 mg, 1.3 mmol, 1.0 eq.) in conc. HCl acid (10 mL) at 0-5° C. The mixture was stirred for an hour at 0-5° C. and then allowed to stir at rt overnight. Water (100 mL) was added to the precipitated product after which it was filtered, washed with water, and vacuum dried to obtain intermediate 9 as a pale yellow solid (291 g, 71%).

¹H NMR (400 MHz, Chloroform-d) δ 3.68 (t, J=6.6 Hz, 2H), 3.09 (s, 4H), 2.72 (t, J=5.9 Hz, 2H), 2.04-1.93 (m, 2H), 1.81-1.72 (m, 2H)

LC-MS (λ=254 nm): 99%, t_{R}=6.8 min. MS (ESI+): 320 [M+H]⁺

### Synthesis of Intermediate 10

To a solution of intermediate 4 (150 mg, 0.47 mmol, 1.0 eq.) in CH₃CN (4 mL) was added K₂CO₃ (300 mg) and 1-Boc-piperazine (219 mg, 1.17 mmol, 2.5 eq.) and the reaction mixture was heated under reflux overnight. Upon completion, the reaction was quenched by addition of 20 mL of sat. NaHCO₃ solution. The aqueous phase was extracted with CH₂Cl₂ (3×20 mL) and the organic layers were combined, washed with brine, dried over anhydrous Na₂SO₄, filtered, and concentrated under reduced pressure to obtain a dark brown crude material. The crude material was adsorbed onto silica gel, and purified by normal phase automated Teledyne Isco chromatography using a CH₂Cl₂/MeOH/NH₃ solvent system. Intermediate 10 was obtained as a white solid (50 mg, 23%).

¹H NMR (400 MHz, Chloroform-d) δ 4.07-3.98 (m, 4H), 3.72 (t, J=6.6 Hz, 2H), 3.62 (dd, J=6.1, 4.3 Hz, 4H), 3.01 (s, 6H), 2.71 (t, J=5.9 Hz, 2H), 2.01-1.89 (m, 2H), 1.76 (m, 2H), 1.48 (s, 9H)

LC-MS (λ=254 nm): 99%, t_{R}=6.9 min. MS (ESI+): 470 [M+H]⁺

### Synthesis of Compound 15

To a solution of intermediate 10 (50 mg, 0.106 mmol) in CH₂Cl₂ (1 mL) at 0° C. was added trifluoroacetic acid (1 mL), and the reaction mixture was stirred until completion at room temperature. The solution was washed with saturated NaHCO₃ solution, dried over anhydrous Na₂SO₄, filtered, and vacuum concentrated to obtain compound 15 as a white solid (35 mg, 90%).

¹H NMR (400 MHz, DMSO-d₆) δ 4.15-4.06 (m, 4H), 3.59 (t, J=6.5 Hz, 2H), 3.29-3.21 (m, 4H), 3.00 (s, 6H), 2.73 (t, J=5.8 Hz, 2H), 1.98-1.87 (m, 2H), 1.77-1.64 (m, 2H)

LC-MS (λ=254 nm): 99%, t_{R}=5.6 min. MS (ESI+): 370 [M+H]⁺

### Example 3

### Synthesis of Compound 18

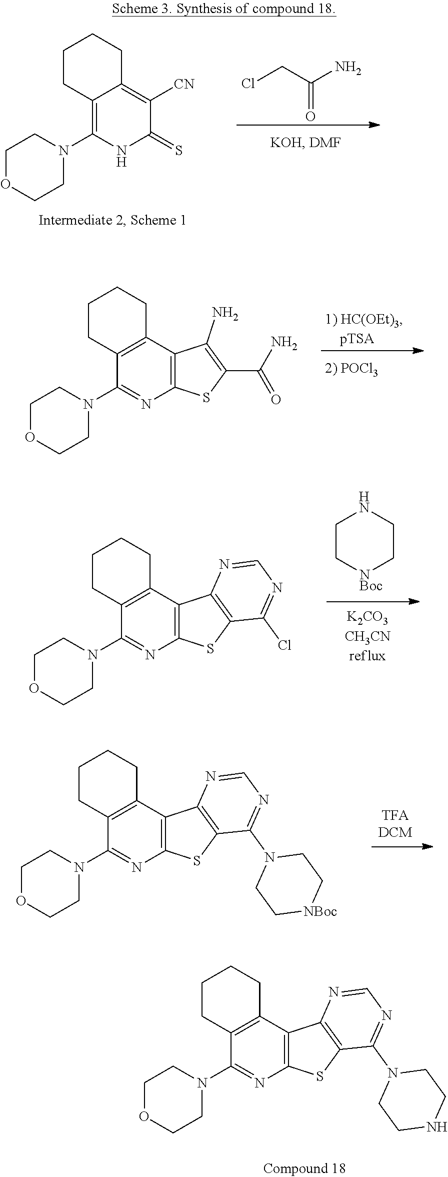

### Example 4

### Synthesis of Compounds 21, 28 and 29

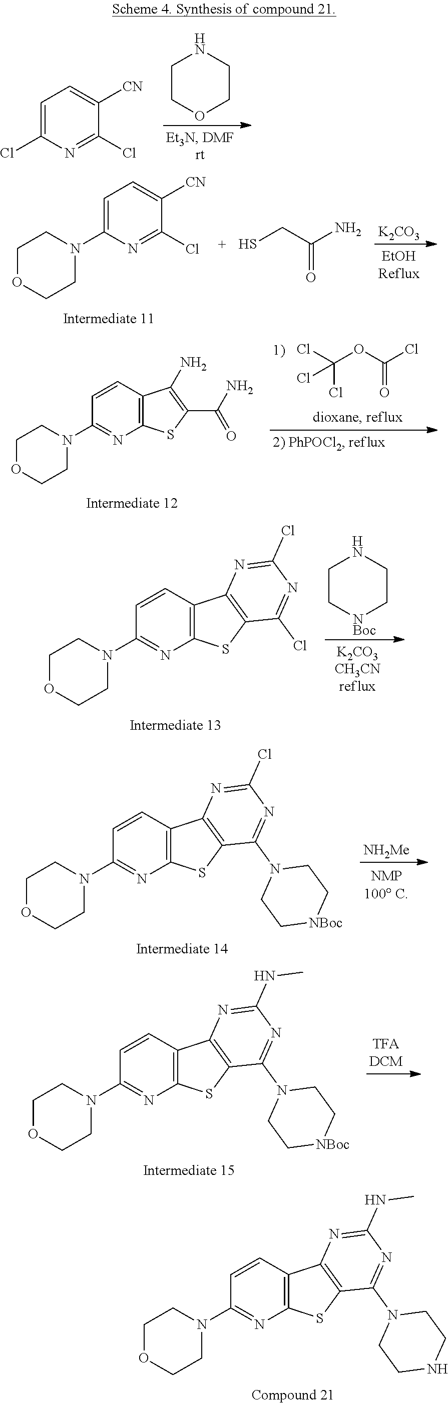

Compounds 28 and 29 in Table 1 were prepared by a similar method as compound 21.

### Synthesis of Intermediate 11

2,6-Dichloropyridine-3-carbonitrile (300 mg, 1.73 mmol, 1.0 eq.) was dissolved in DMF (2 mL) in a flame dried round bottom flask under nitrogen atmosphere. To the resultant solution was added morpholine (0.15 mL, 1.73 mmol, 1.0 eq.), and triethylamine (0.5 mL, 3.46 mmol, 2.0 eq.). The reaction was stirred overnight at room temperature under nitrogen. The reaction was quenched with saturated aqueous NH₄Cl solution (10 mL). The aqueous phase was separated and extracted with EtOAc (5×10 mL). All combined organic layers were dried over anhydrous Na₂SO₄, filtered, and concentrated under reduced pressure to obtain a white crude material. ¹H NMR and LC-MS of the crude material showed the presence both mono- and di-amination products (6:1, same t_{R}). The crude material was used in the next reaction without further purification (350 mg).

### Synthesis of Intermediate 12

Crude intermediate 11 (350 mg) was dissolved in ethanol (9 mL) in a flame dried round bottom flask under an atmosphere of nitrogen. To the resultant solution was added 2-mercaptoacetamide (100 mg/1 mL in methanolic ammonia solution, 1.71 mL, 1.88 mmol) and anhydrous K₂CO₃ (521 mg, 3.8 mmol). The reaction was refluxed overnight at 100° C. under nitrogen. The reaction was quenched with water (50 mL) which precipitated the desired product. The precipitated product was filtered, washed with water (50 mL), and concentrated under reduced pressure to obtain intermediate 12 as a pale yellow solid (299 mg, 68% over two steps).

¹H NMR (400 MHz, DMSO-d₆) δ 8.11 (d, J=9.1 Hz, 1H), 7.03 (br s, 2H), 6.93 (d, J=9.1 Hz, 1H), 6.82 (br s, 2H), 3.73-3.67 (m, 4H), 3.59-3.53 (m, 4H)

LC-MS (λ=254 nm): 99%, t_{R}=4.6 min. MS (ESI+): 279 [M+H]⁺

### Synthesis of Intermediate 13

Intermediate 12 (150 mg, 1.56 mmol, 1.0 eq.) was dissolved in dioxane (2 mL) in a flame dried round bottom flask under nitrogen atmosphere. To the resultant solution was added trichloromethyl chloroformate (0.07 mL, 0.61 mmol, 1.1 eq.), and the reaction was refluxed for 3 hours at 102° C. under nitrogen. The reaction was slowly quenched with water (10 mL). The precipitated product was filtered, washed with water (50 mL), and concentrated under reduced pressure to obtain a yellow solid (118 mg, 69%).

¹H NMR (400 MHz, DMSO-d₆) δ 12.03 (br s, 1H), 11.29 (br s, 1H), 8.33 (d, J=9.3 Hz, 1H), 7.10 (d, J=9.3 Hz, 1H), 3.70 (m, 4H), 3.68-3.60 (m, 4H)

LC-MS (λ=254 nm): 99%, t_{R}=5.0 min. MS (ESI+): 305 [M+H]⁺

The above yellow solid (110 mg, 0.36 mmol, 1.0 eq.) was added to phenylphosphonic dichloride (4 mL) in a flame dried round bottom flask under nitrogen atmosphere. The reaction was refluxed for 2 hours at 170° C. under nitrogen. The reaction was slowly quenched with water (10 mL) which precipitated the desired product. The precipitated product was filtered, washed with more water (30 mL), and concentrated under reduced pressure to obtain intermediate 13 as a yellow solid (>30 mg, >25%).

¹H NMR (400 MHz, Chloroform-d) δ 8.34 (d, J=9.1 Hz, 1H), 6.80 (d, J=9.1 Hz, 1H), 3.85-3.75 (m, 8H)

LC-MS (λ=254 nm): 99%, t_{R}=5.6 min. MS (ESI+): 342 [M+H]⁺

### Synthesis of Intermediate 14

To a solution of intermediate 13 (90 mg, 0.088 mmol, 1.0 eq.) in CH₃CN (2 mL) was added K₂CO₃ (300 mg) and 1-Boc-piperazine (82 mg, 0.43 mmol, 5.0 eq.), and the reaction mixture was heated under reflux overnight. Upon completion, the reaction was quenched by addition of 20 mL of sat. NaHCO₃ solution. The aqueous phase was extracted with CH₂Cl₂ (3×20 mL) and the organic layers were combined, washed with brine, dried over anhydrous Na₂SO₄, filtered, and concentrated under reduced pressure to obtain a dark brown crude material. The crude material was adsorbed onto silica gel and purified by normal phase automated Teledyne Isco chromatography using a CH₂Cl₂/MeOH/NH₃ solvent system. Intermediate 14 was obtained as a pale yellow solid (40 mg, 93%).

¹H NMR (400 MHz, Chloroform-d) δ 8.31 (d, J=9.0 Hz, 1H), 6.76 (d, J=9.1 Hz, 1H), 3.97-3.90 (m, 4H), 3.85-3.77 (m, 4H), 3.73-3.65 (m, 4H), 3.58 (m, 4H), 1.47 (s, 9H)

LC-MS (λ=254 nm): 99%, t_{R}=6.6 min. MS (ESI+): 492 [M+H]⁺

### Synthesis of Intermediate 15

Intermediate 14 (110 mg, 0.36 mmol, 1.0 eq.) was added to methylamine (2M in THF, 5 mL) in a flame dried sealed tube under nitrogen atmosphere. The reaction was heated for 5 days at 100° C. The reaction was then quenched with water (15 mL) and extracted with ethyl acetate (3×20 mL). The combined organic fractions were washed with sat. NaHCO₃ solution, dried over anhydrous Na₂SO₄, filtered, concentrated under reduced pressure, and purified by reverse phase automated Teledyne Isco chromatography using MeOH/H₂O/0.1% acetic acid solvent system. Intermediate 15 was obtained as an orange solid (74 mg, 54%).

¹H NMR (400 MHz, Chloroform-d) δ 9.50 (br s, 1H), 8.61 (d, J=8.4 Hz, 1H), 6.82 (d, J=9.3 Hz, 1H), 4.05 (dd, J=6.1, 4.3 Hz, 4H), 3.83-3.78 (m, 4H), 3.73-3.69 (m, 4H), 3.65-3.60 (m, 4H), 2.99 (d, J=3.5 Hz, 3H), 1.23 (s, 9H)

LC-MS (λ=254 nm): 99%, t_{R}=5.6 min. MS (ESI+): 487 [M+H]⁺

### Synthesis of Compound 21

To a solution of intermediate 15 (30 mg, 0.06 mmol) in CH₂Cl₂ (2 mL) at 0° C. was added trifluoroacetic acid (2 mL), and the reaction mixture was stirred until completion at room temperature. The solution was washed with saturated NaHCO₃ solution, dried over anhydrous Na₂SO₄, filtered, and vacuum concentrated to obtain compound 21 as a white solid (10 mg, 10%).

¹H NMR (400 MHz, DMSO-d₆) δ 8.93 (br s, 1H), 8.23 (d, J=9.1 Hz, 1H), 7.08 (d, J=9.1 Hz, 1H), 4.02 (s, 4H), 3.68 (m, 8H), 3.29 (s, 4H), 2.89 (s, 3H)

LC-MS (λ=254 nm): 99%, t_{R}=4.2 min. MS (ESI+): 387 [M+H]⁺

### Example 5

### Synthesis of Compound 20

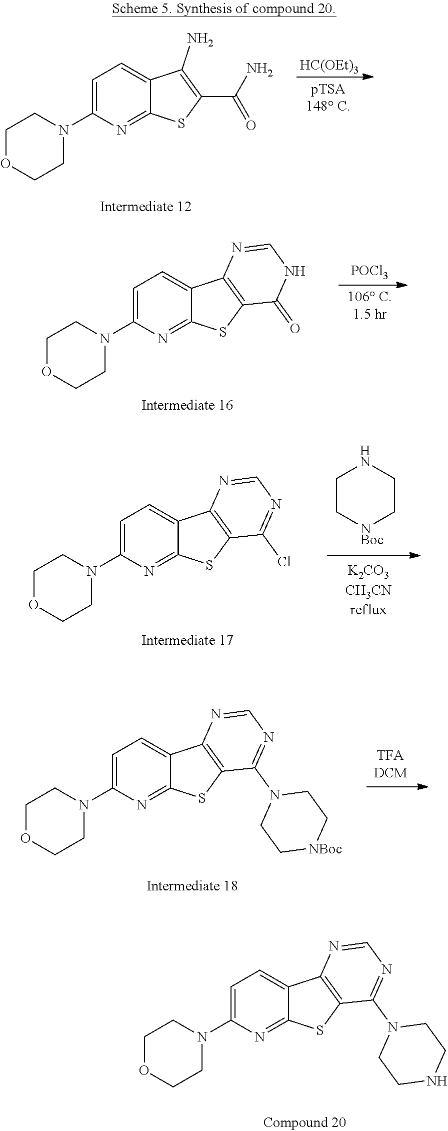

### Example 6

### Synthesis of Compound 19

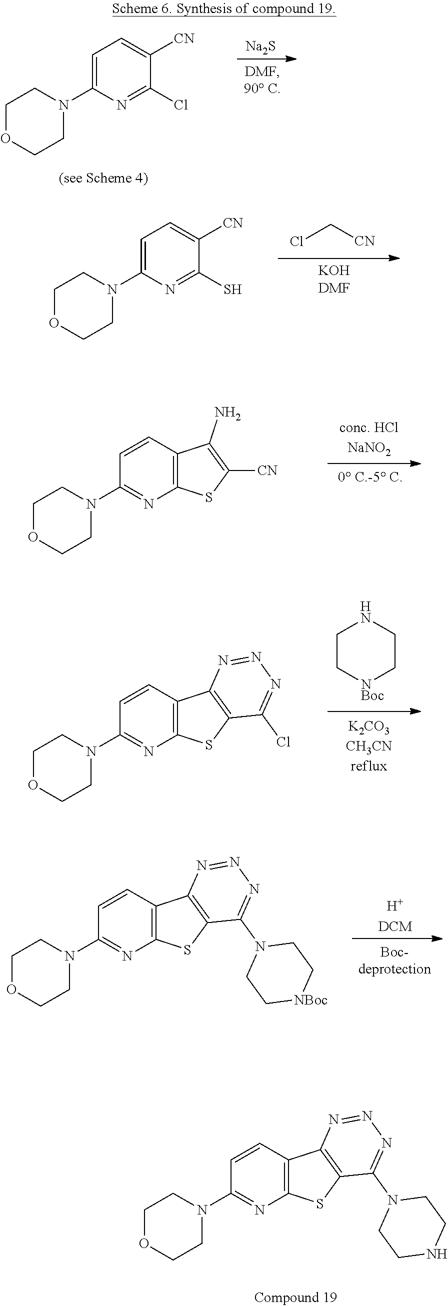

Compound 27 in Table 1 was prepared by a similar method as compound 19.

### Synthesis of Intermediate 16

Intermediate 12 (150 mg, 0.54 mmol, 1.0 eq.) was added to triethyl orthoformate (5 mL, excess) followed by p-toluenesulfonic acid monohydrate (10.2 mg, 0.05 mmol, 0.1 eq.) in a flame dried round bottom flask under nitrogen atmosphere. The reaction was refluxed overnight at 148° C. under nitrogen. The reaction was quenched with water (15 mL) and extracted with ethyl acetate (3×20 mL). The combined organic fractions were washed with saturated NaHCO₃ solution, dried over anhydrous Na₂SO₄, filtered, and concentrated under reduced pressure to obtain a yellow product which was used in the next reaction without further purification (155 mg, quantitative).

¹H NMR (400 MHz, DMSO-d₆) δ 8.25 (s, 1H), 8.22 (d, J=9.1 Hz, 1H), 7.12 (d, J=9.1 Hz, 1H), 3.69 (m, 8H)

LC-MS (λ=254 nm): 99%, t_{R}=5.1 min. MS (ESI+): 289 [M+H]⁺

### Synthesis of Intermediate 17

Intermediate 16 (150 mg, 0.52 mmol, 1.0 eq.) was added to phosphoryl chloride (3 mL, excess) in a flame dried round bottom flask under nitrogen atmosphere. The reaction was refluxed for 90 minutes at 106° C. The reaction was cooled and concentrated under reduced pressure to obtain a dark black crude material. This crude material was used in the next reaction without further purification (158 mg, quantitative).

¹H NMR (400 MHz, Chloroform-d) δ 8.95 (s, 1H), 8.47 (d, J=9.1 Hz, 1H), 6.85 (d, J=9.1 Hz, 1H), 3.88-3.79 (m, 8H)

LC-MS (λ=254 nm): 99%, t_{R}=6.1 min. MS (ESI+): 308 [M+H]⁺

### Synthesis of Intermediate 18

To a solution of intermediate 17 (150 mg, 0.5 mmol, 1.0 eq.) in CH₃CN (7 mL) was added K₂CO₃ (300 mg) and 1-Boc-piperazine (455 mg, 2.4 mmol, 5.0 eq.) and the reaction mixture was heated under reflux overnight. Upon completion, the reaction was quenched by addition of 20 mL of sat. NaHCO₃ solution. The aqueous phase was extracted with CH₂Cl₂ (3×20 mL) and the organic layers were combined, washed with brine, dried over anhydrous Na₂SO₄, filtered, and concentrated under reduced pressure to obtain a dark brown crude material. The crude material was adsorbed onto silica gel and purified by normal phase automated Teledyne Isco chromatography using a CH₂Cl₂/MeOH/NH₃ solvent system. Intermediate 18 was obtained as a yellow solid (39 mg, 17%).

¹H NMR (400 MHz, Chloroform-d) δ 8.61 (s, 1H), 8.32 (d, J=9.0 Hz, 1H), 6.77 (d, J=9.0 Hz, 1H), 3.95-3.88 (m, 4H), 3.84-3.66 (m, 8H), 3.61-3.54 (m, 4H), 1.47 (s, 9H)

LC-MS (λ=254 nm): 99%, t_{R}=6.3 min. MS (ESI+): 457 [M+H]⁺

### Synthesis of Compound 20

To a solution of intermediate 18 (39 mg, 0.08 mmol) in CH₂Cl₂ (2 mL) at 0° C. was added trifluoroacetic acid (2 mL) and the reaction mixture was stirred until completion at room temperature. The solution was washed with saturated NaHCO₃ solution, dried over anhydrous Na₂SO₄, filtered, and vacuum concentrated to obtain compound 20 as a pale yellow solid (29 mg, 99%).

¹H NMR (400 MHz, DMSO-d₆) δ 8.92 (br s, 1H), 8.67 (s, 1H), 8.34 (d, J=9.1 Hz, 1 H), 7.14 (d, J=9.1 Hz, 1H), 4.11-4.05 (m, 4H), 3.75-3.67 (m, 8H), 3.30 (s, 4H)

LC-MS (λ=254 nm): 99%, t_{R}=6.6 min. MS (ESI+): 357 [M+H]⁺

### Example 7

### Synthesis of Compound 22

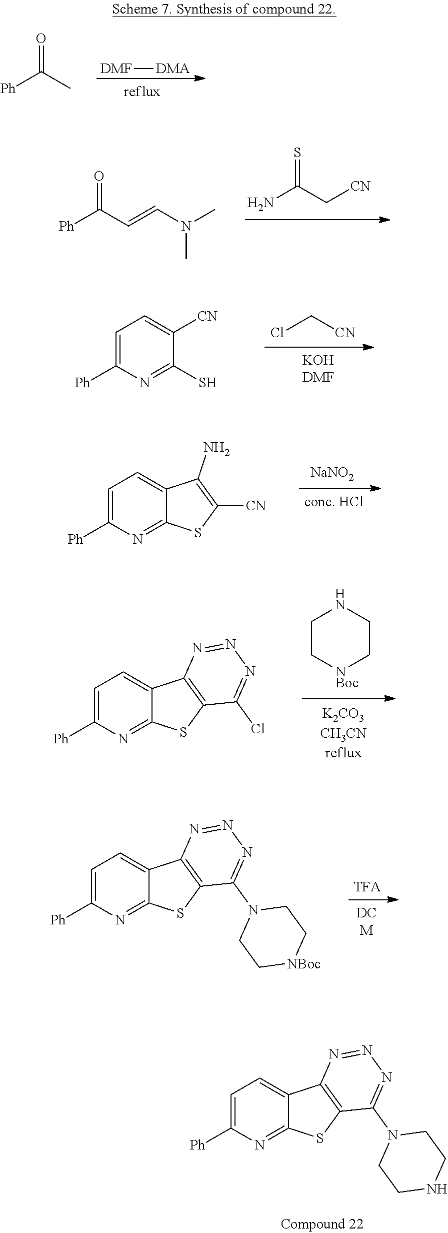

### Example 8

### Synthesis of Compounds 24-26

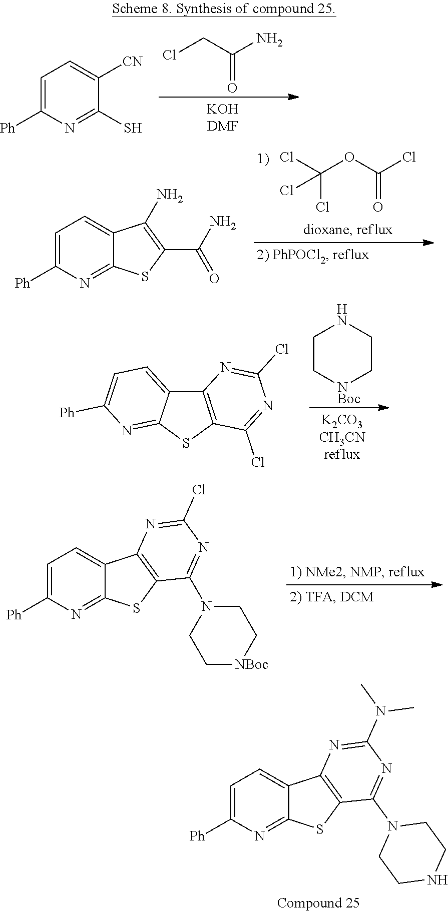

Compounds 24 and 26 in Table 1 were prepared by a similar method as compound 25.

### Example 9

### Synthesis of Compound 23

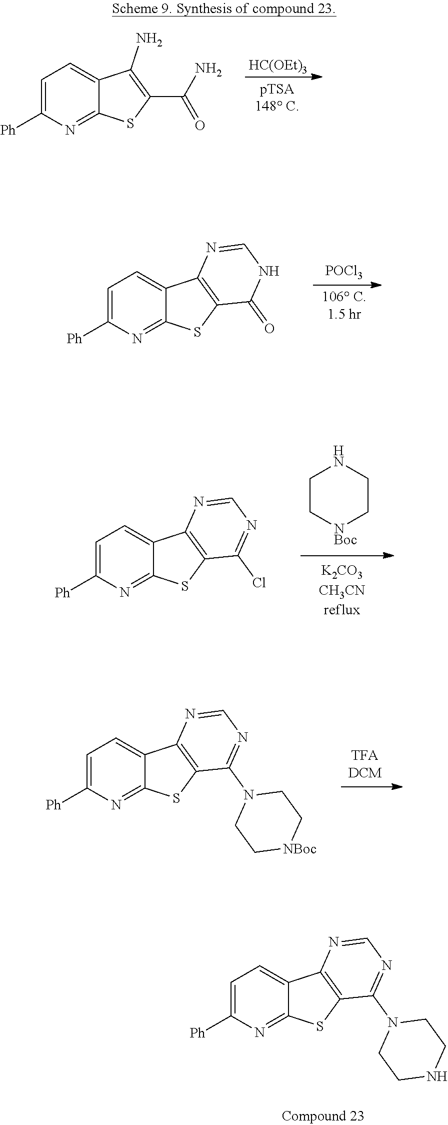

### Example 10

### Synthesis of Compound 30

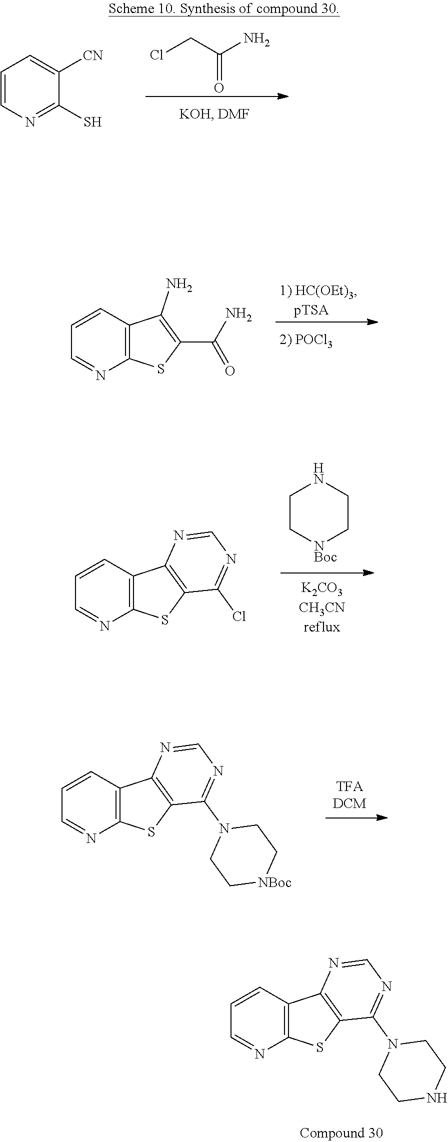

### Example 11

### Synthesis of Compound 31

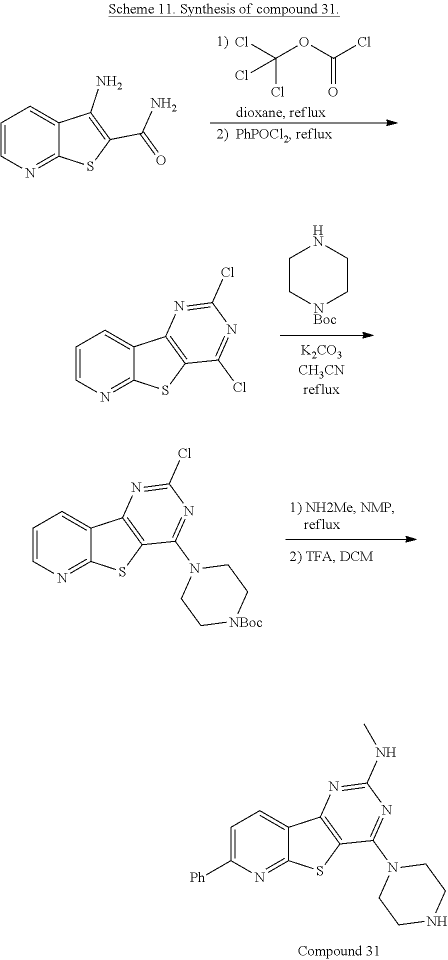 [SMILES: NC(=O)c1sc2ncccc2c1N ; O=C(Cl)OC(Cl)(Cl)Cl ; Clc1nc(Cl)c2sc3ncccc3c2n1 ; CC(C)(C)OC(=O)N1CCNCC1 ; CC(C)(C)OC(=O)N1CCN(c2nc(Cl)nc3c2sc2ncccc23)CC1 ; CNc1nc(N2CCNCC2)c2sc3nc(-c4ccccc4)ccc3c2n1]

B. Results of Formula I Compounds

### Example 12

### In Vitro Binding Assay

To assess GUS inhibition, cleavage of p-nitrophenyl-β-D-glucuronide (PNPG) by purified E. coli GUS in the presence of inhibitor was measured. The reaction was performed in 96-well clear-bottom plates at a volume of 50 μL. Each reaction contained 5 μL of 100 nM E. coli GUS, 5 μL of inhibitor at various concentrations, 10 μL of 250 mM HEPES and 250 mM NaCl assay buffer, and 30 μL of 1.5 mM PNPG. The reaction was initiated by addition of PNPG and incubated at 37° C. for 2 hours. End point absorbance values at 410 nm were measured using a PHERAstar Plus microplate reader. The resulting data were fit with a four-parameter logistic log function in SigmaPlot 13.0 to determine IC₅₀ values. IC₅₀ was defined as the concentration of inhibitor that yields a 50% reduction in product formation at equilibrium.

TABLE 1
Data for in vitro potency against E. coli (EcGUS).

| Compound | |
|---|---|
| No. | EcGUS IC₅₀ (nM) |
| 2 | 37600 ± 700 |
| 3 | 5000 ± 500 |
| 4 | 30000 ± 10000 |
| 5 | 9000 ± 2000 |
| 6 | 2400 ± 400 |
| 7 | 2400 ± 300 |
| 8 | 9000 ± 2000 |
| 9 | 5600 ± 500 |
| 10 | NI |
| 11 | NI |
| 12 | 130 ± 10 |
| 13 | 500 ± 200 |
| 14 | 800 ± 200 |
| 15 | 130 ± 10 |
| 16 | 70 ± 10 |
| 17 | 190 ± 40 |
| 19 | 170 ± 40 |
| 20 | 130 ± 10 |
| 21 | 100 ± 20 |
| 22 | 150 ± 50 |
| 23 | 130 ± 30 |
| 24 | 180 ± 90 |
| 25 | 190 ± 90 |
| 26 | 120 ± 20 |
| 27 | 80 ± 30 |
| 28 | 42 ± 6 |
| 29 | 40 ± 8 |
| 30 | 80 ± 20 |
| 31 | 36 ± 2 |
| 32 | NI |
| 34 | NI |
| 35 | NI |
| 36 | NI |
| 37 | NI |
| 38 | NI |
| 39 | NI |
| 40 | NI |
| 41 | NI |
| 42 | NI |
| 43 | NI |
| 44 | 5400 ± 600 |
| 45 | 140 ± 8 |
| 46 | 1400 ± 40 |
| 47 | 30 ± 3 |
| 48 | 208 ± 3 |
| 49 | 620 ± 50 |
| 50 | 12000 ± 2000 |
| 51 | 900 ± 60 |

NI = no inhibition; Reference compound Inh9 = IC50 of 380 ± 50.

### Example 13

### Determination of how β-glucuronidase Inhibitors Impact Chemotherapeutic Efficacy—Approach 1

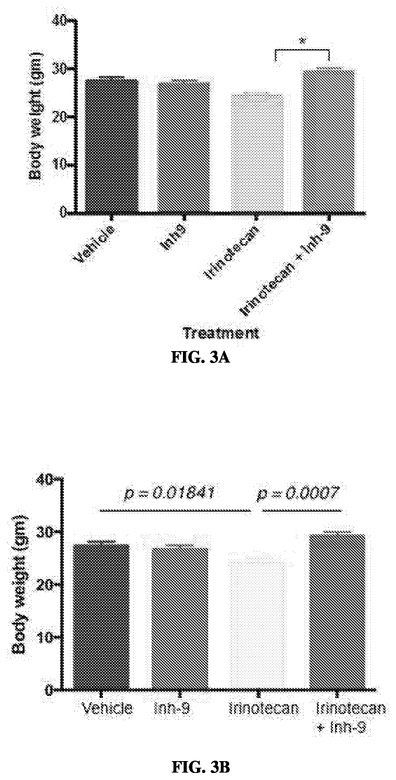

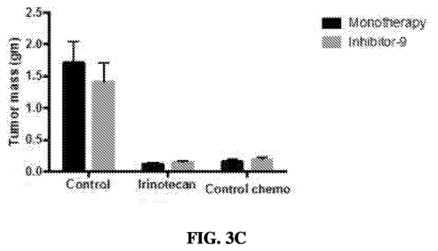

The approach uses xenografts in athymic nude mice and the results are shown in FIGS. 3A-C, 4, and 5A-B. The steps consist of:
Sum149 breast cancer lines are cultured.
The cells are injected subcutaneously in the hind flank of athymic nude mice.
One group of mice is treated only with chemotherapy (irinotecan).
Another group of mice is treated with chemotherapy and Inh9.
The groups of mice are monitored regularly for GI symptoms.
One alternate days:

Fecal samples collected alternate days

Tumor volumes measured alternate days
On sac day:

measure tumors

excise tumors, preserve

measure spleen weight, preserve

excise large intestine; swiss roll tissue prep

collect fecal & cecal contents; preserve cecum

histopathological analysis after tissue fixation and relevant staining

### Example 14

### Determination of how β-glucuronidase Inhibitors Impact Chemotherapeutic Efficacy—Approach 2

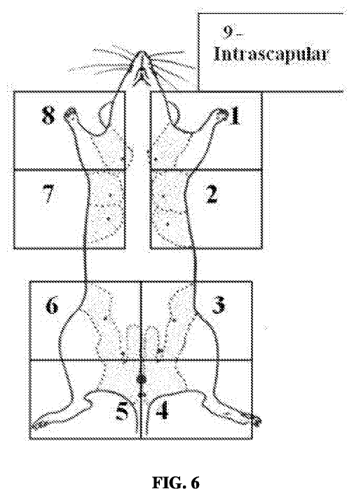

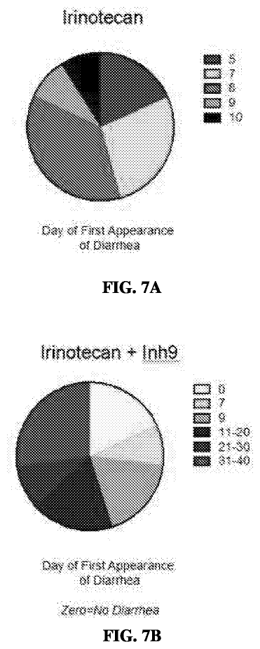

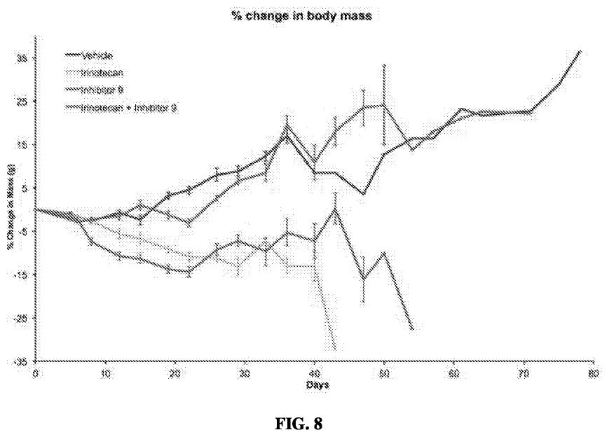

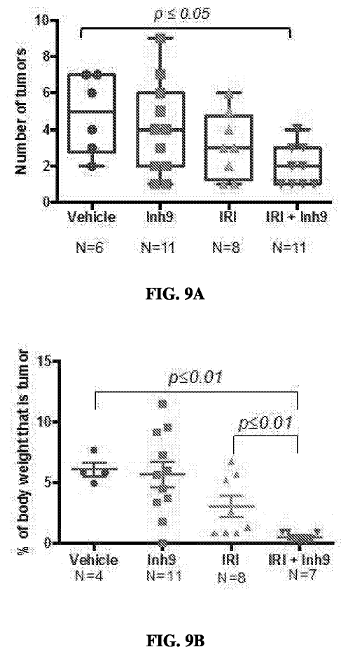

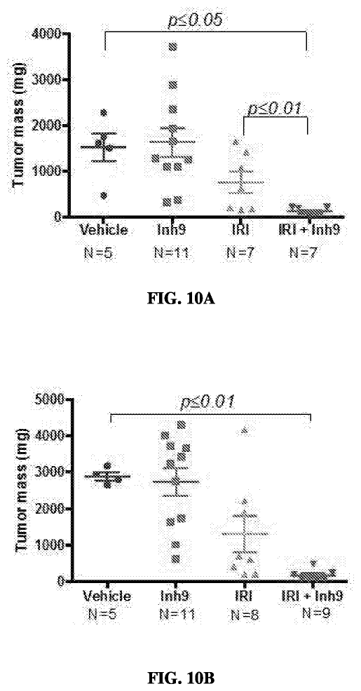

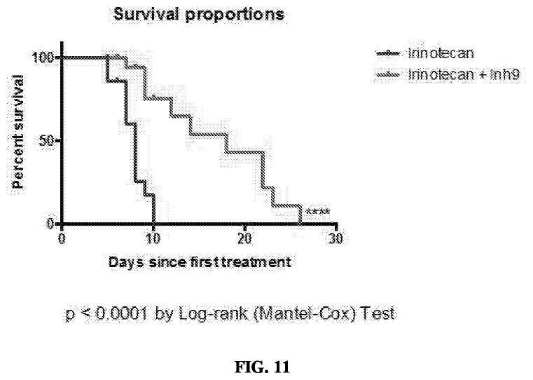

The approach uses immune competent genetically engineered mouse models and the results are disclosed in FIGS. 7A-B, 8, 9A-B, 10A-B and 11. Also, the regions of possible tumors in the C3-Tag mouse model (female mice) are disclosed in FIG. 6.

### C3-Tag Mice

Strain of Origin: FVB/N
Promoter: C3(1) Prostatic binding protein C3 promoter
Gene inserted: SV40 large T antigen
Tumors develop in mammary tissue of female mice

### Dosing Scheme:

Week 1: 2 treatments per week
Week 2: 3 treatments per week
Week 3: 3 treatments per week
Week 4: 5 treatments per week

### Doses:

Vehicle→saline, saline+DMSO
Inhibitor-9→10 ug p.o. (in saline) b.i.d, weekdays only
Irinotecan→50 mg/kg i.p., q.d., weekdays only
Animals housed 2 per cage, with 5 cages per treatment group.

### Example 15

### GUS Loop Architecture Dictates SN-38-G Processing Efficiency

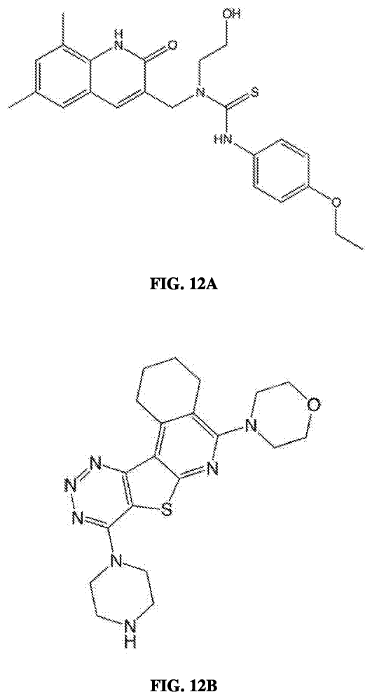

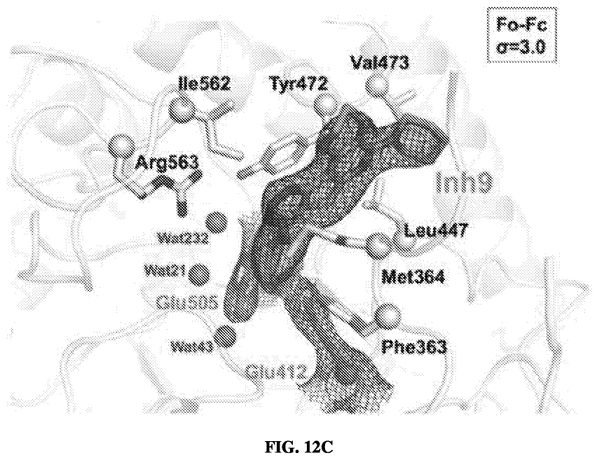

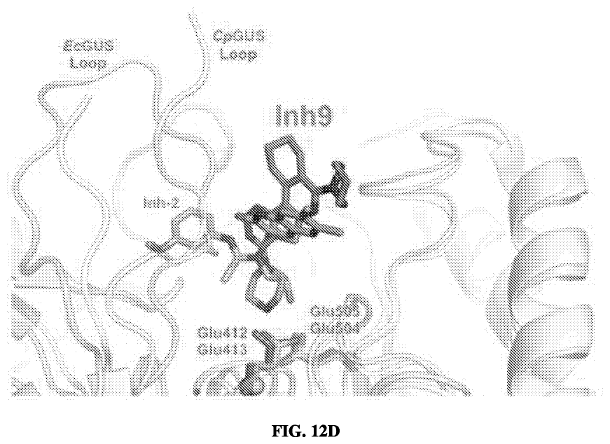

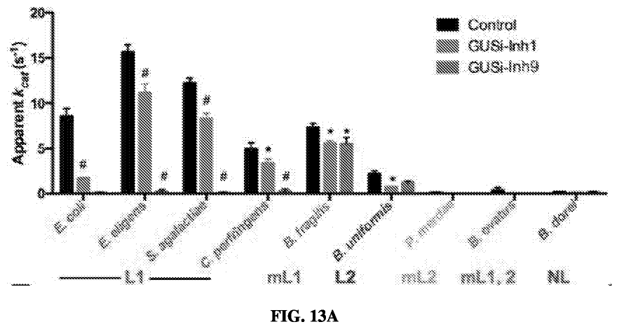

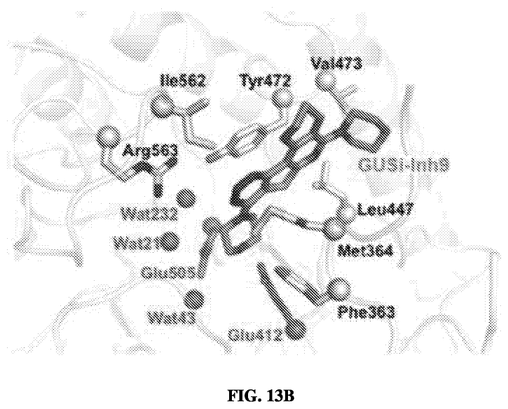

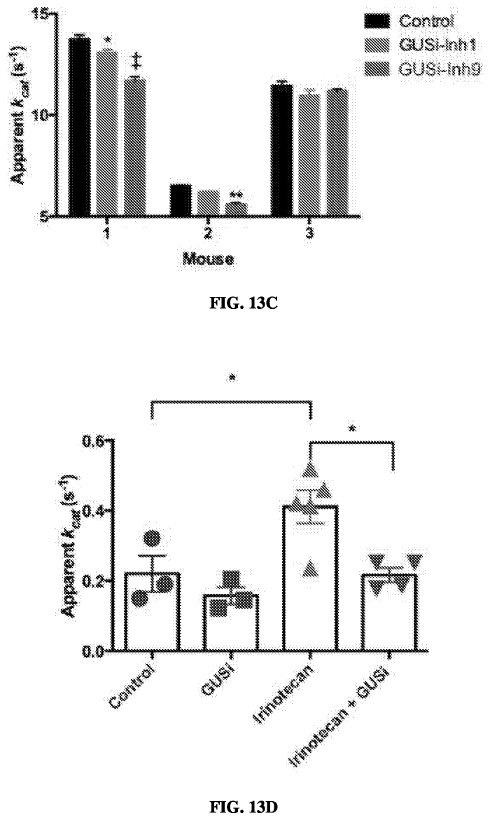

Studies were performed on the SN-38-G cleavage ability of a panel of purified GUS enzymes present in the human gut microbiota, and the inhibition of these proteins by two potent, selective and non-lethal chemotypes: Inh1 (FIG. 12A), which has been employed in a range of in vitro and animal studies (Wallace 2010, Ahmad 2011 PMC3106358, Roberts 2013 PMC3716326; et al, 2014 PMID 24160697; Boelsterli et al, 2013 PMID 23091168), and Inh9, a distinct chemotype (FIG. 12B). Without inhibitors, it was found that of the panel of enzymes examined, Loop 1 (L1) GUS enzymes process SN-38-G most efficiently, particularly those from the Proteobacterium E. coli and the Firmicutes Eubacterium eligens and Streptococcus agalactiae (FIG. 13A). It was also found that the Mini-Loop 1 (mL1) GUS from Bacteroides fragilis exhibited SN-38-G processing activity, as did the Loop 2 (L2) enzyme from Bacteroides uniformis, although to a lesser degree (FIG. 13A). The remainder of the GUS enzymes examined did not exhibit SN-38-G processing activities (FIG. 13A). These data correlate with our previous results showing that L1 GUS proteins most efficiently process the small, standard GUS substrate p-nitrophenyl-glucuronide (PNPG) substrate, and that mL1 and L2 GUS enzymes are also able to perform this reaction (Pollet et al., Structure, 2017). By contrast, the remainder of the GUS enzymes tested, mL2, mL1,2 and NL, do not process SN-38-G efficiently (FIG. 13A), similar to previous data using PNPG.

Inh9 was found to be superior to Inh1 in inhibiting GUS-mediated reactivation of SN-38-G in vitro (FIG. 13A). Inh1 demonstrated strong activity against E. coli GUS, but weak effects on other GUS enzymes, including others from the L1 category of proteins (FIG. 13A). Inh9 efficiently reduced apparent SN-38-G processing kcat values of the four L1 enzymes tested, those from E. coli, E. eligens, S. agalactiae and C. perfringens. This inhibition includes the enzymes found to process SN-38-G most efficiently. While Inh9 did not inhibit the B. fragilis mL1 or B. uniformis L2 proteins (FIG. 13A), Inh9 potently reduced in vitro SN-38-G reactivation by gut microbial L1 GUS enzymes.

### Example 16

### Crystal Structure of Inh9-GUS Complex

To understand the structural basis of the inhibition of gut microbial GUS enzymes by Inh9, a 2.7 Å resolution crystal structure of this compound bound to the L1 GUS from C. perfringens was generated. Inh9 binds to the active site of the enzyme and directly contacts the catalytic and negatively-charged active site residues E412 and E505 using its positively-charged piperizine secondary amine (FIG. 12C; FIG. 13B). The planar and aromatic region of Inh9 forms a π-stacking interaction with the aromatic side chain of Y572, and hydrophobic contacts with F363, M364, L447, M448, V473, and I562 (FIG. 13B). The catalytic residues and the adjacent tyrosine are completely conserved in microbial GUS enzymes (Wallace et al 2016; Pollet et al 2017). Inh9 binds in a similar location in C. perfringens GUS to that observed for the Inh1 analog Inh2 when bound to E. coli GUS, but employs an electrostatic contact with the anionic catalytic residues that is missing with the Inh1 scaffold (FIG. 12D). Thus, without being bound by theory, it is believed that Inh9 binds at the active site of microbial GUS enzymes using a combination of electrostatic and aromatic contacts conserved in GUS proteins from the gut microbiome.

### Example 17

### SN-38-G Processing by In Fimo Samples

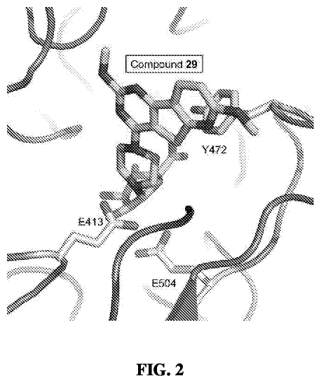

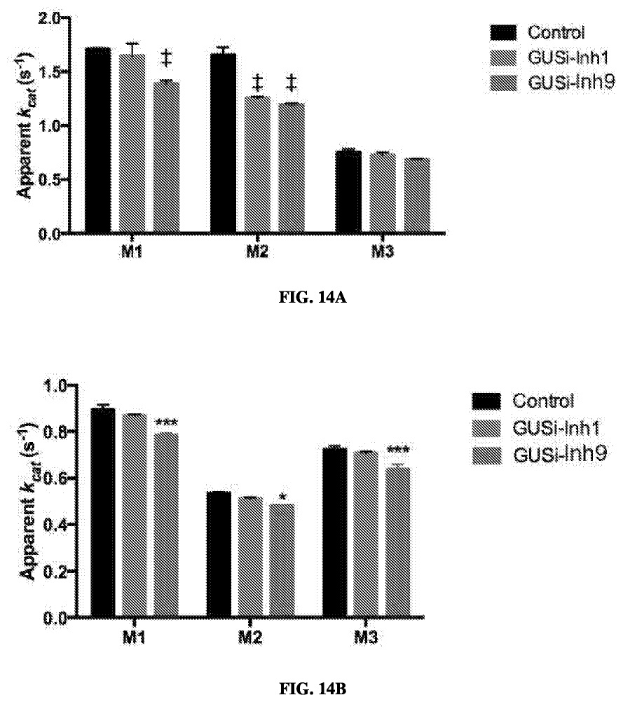

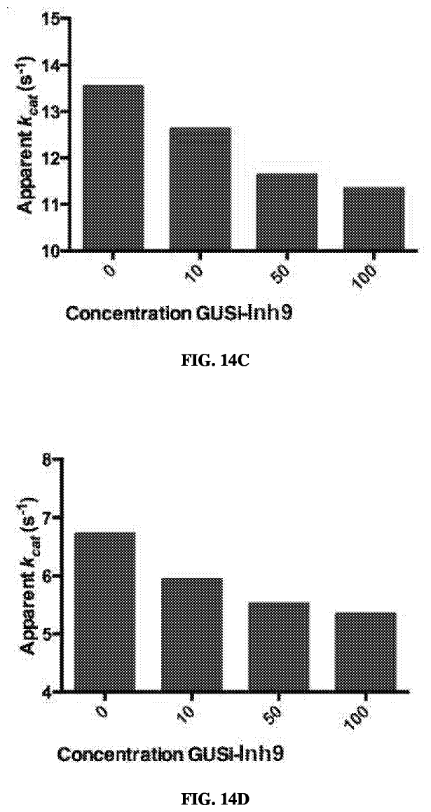

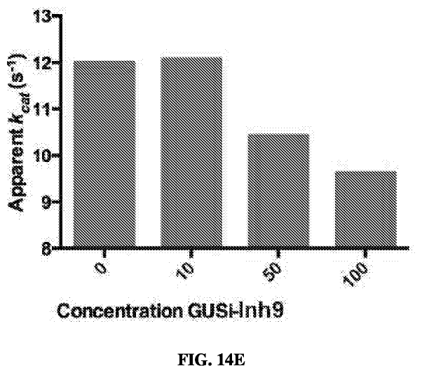

To examine the activity of a more relevant sample of gut microbial GUS proteins, intestinal contents from naïve FVB mice in SPF conditions were obtained. The active enzymes mixtures were extracted from these samples and the ability of these mixtures to cleave SN-38-G were tested, as well as the inhibition of this cleavage using Inh1 or Inh9. “Feces” entered English usage in the 17^{th} century and arose from the Latin word faex, meaning “dregs”. As used herein, the ex vivo intestinal samples examined scientifically are to be termed “in fimo” from “fimus”, used by Virgil and in formal Latin to mean “excrement”. The in fimo studies were conducted and revealed that the enzymes present in intestinal contents are able to process SN-38-G, albeit less robustly than purified proteins. In vitro, apparent k_{cat} values of 5-15 sec⁻¹ were observed (FIG. 13A), while the in fimo samples from the cecum, proximal colon and distal colon exhibited apparent k_{cat} values of 6-12 sec⁻¹ (cecal), 0.7-1.6 sec⁻¹, and 0.5-0.9 sec⁻¹, respectively (FIG. 13C; FIGS. 14A and 14B). More robust SN-38-G processing was observed in the cecum, followed by the proximal colon, while the distal colon exhibited the weakest processing of this substrate. It was found that GUS inhibition was more effective in samples from gut compartments that showed the greatest SN-38-G cleavage ability (FIG. 13C; FIGS. 14A and 14B). Akin to the in vitro results presented above, Inh9 was superior to Inh1 in reducing SN-38-G processing in fimo (FIG. 13C; FIGS. 14A and 14B). Inh9 at 50 and 100 μM were found to significantly reduce SN-38-G reactivation by cecal contents (FIGS. 2C-2E). These results indicate that complex protein mixtures obtained from mouse intestinal contents show moderate SN-38-G reactivation capabilities that differ depending on intestinal location. They further show that the Loop 1-targeted GUS inhibitors employed are able to reduce SN-38-G reactivation, with Inh9 exhibiting superior efficacy than Inh1.

The FVB mice were treated with irinotecan, the Inh9, or both irinotecan and Inh9, and it was determined how these treatments affected SN-38-G reactivation in in fimo samples collected after 24 hours. It was foung found that a single 50 mg/kg ip irinotecan treatment significantly increased SN-38-G processing activity in the mouse cecum (FIG. 13D), indicating that irinotecan causes an increase in gut microbial enzyme activity in vivo. It was found that 20 μg po of Inh9 alone did not alter the ability of in fimo samples to reactivate SN-38-G (FIG. 13D). However, Inh9 plus irinotecan significantly reduced the level of SN-38-G reactivation by in fimo samples compared to irinotecan treatment alone, with Inh9 reducing it to levels seen in naïve mice (FIG. 13D). A similar study was conducted in which samples were collected at 120 hours; at this five-day timepoint, no difference was found between the four treatment groups (data not shown). Taken together, these results indicate that irinotecan increases the activity of SN-38-G reactivation by gut microbial enzymes in vivo at 24 hours, and that Inh9 is effectively able to blunt this increased reactivation. By 120 hours, the effects of irinotecan and Inh9 are no longer detected.

### Example 18

### GUS Inhibition Protects Against Gut Damage In Vivo

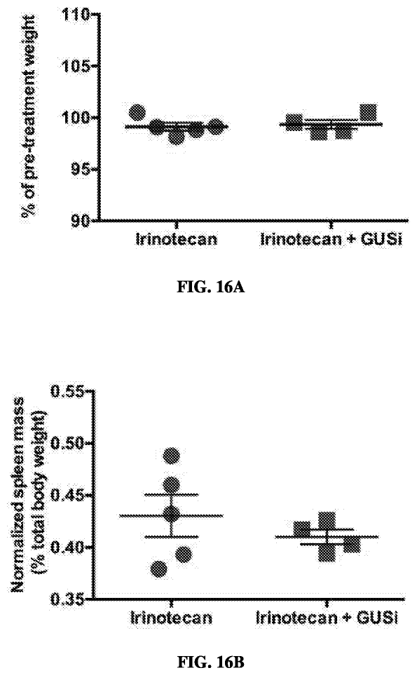

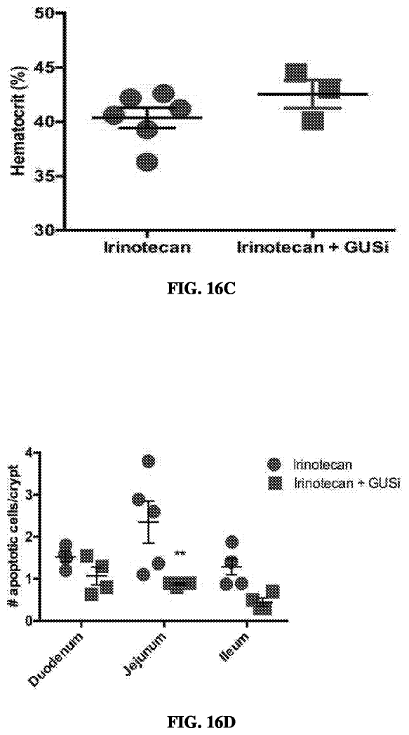

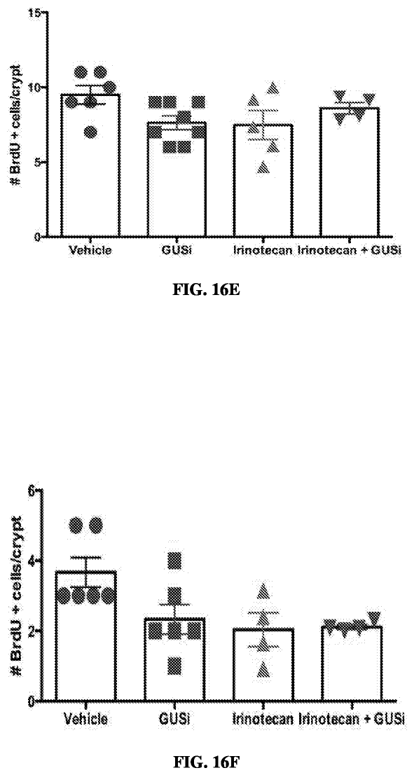

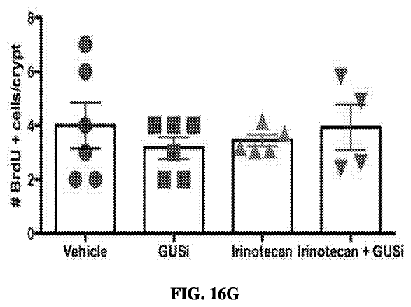

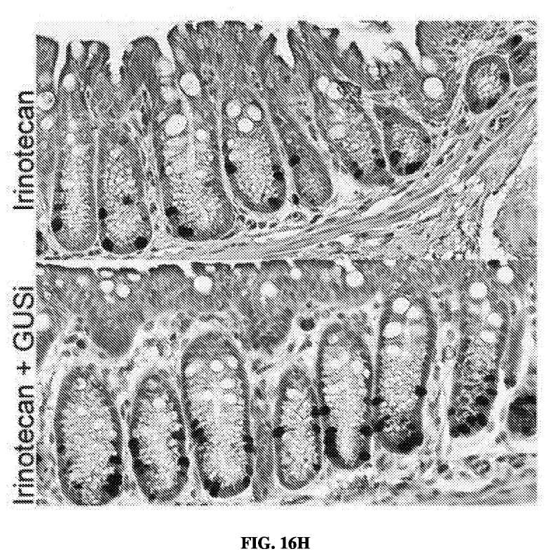

It has been previously reported that irinotecan induces apoptosis and reduces the number of mitotic structures in the rat intestinal epithelium (Takasuna 1996). To assess whether Inh9 impacts cell proliferation in the murine intestinal epithelium, we undertook two time-course studies. Four groups of age-, weight- and litter-matched female FVB mice were treated with a single treatment of vehicle, Inh9 (1 mg/kg p.o.), irinotecan (50 mg/kg i.p.) or irinotecan and Inh9. One set of animals were euthanized after 24 hours, and a second set after 120 hours. To assess overall effects on the animals, changes in body weight, spleen mass and hematocrit were measured. In the GI tract, levels of apoptotic cells and proliferative cells were measured and epithelial samples were examined by H&E staining. At 24 hours, no overall changes were observed in body weight, spleen mass or hematocrit (FIGS. 16A-16C). While Inh9 afforded a modest protection against increases in apoptotic cells per crypt at 24 hours (FIG. 16D), no change in numbers of proliferative cells in the GI tract, as assessed using immunohistochemical detection of BrdU injected 30 min prior to euthanasia, were observed between treatment groups at 24 hours (FIGS. 16E-16H).

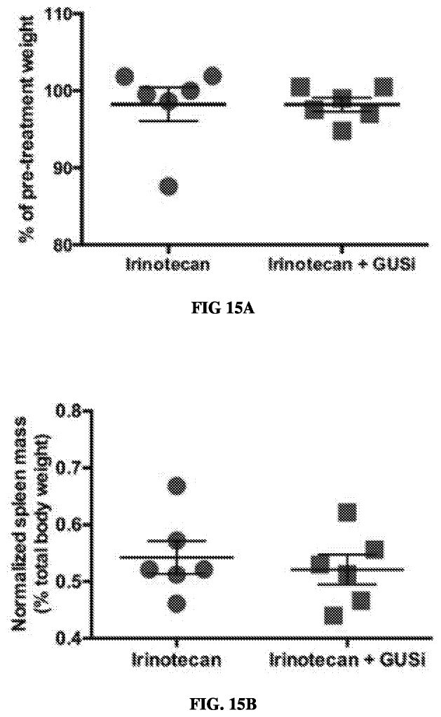

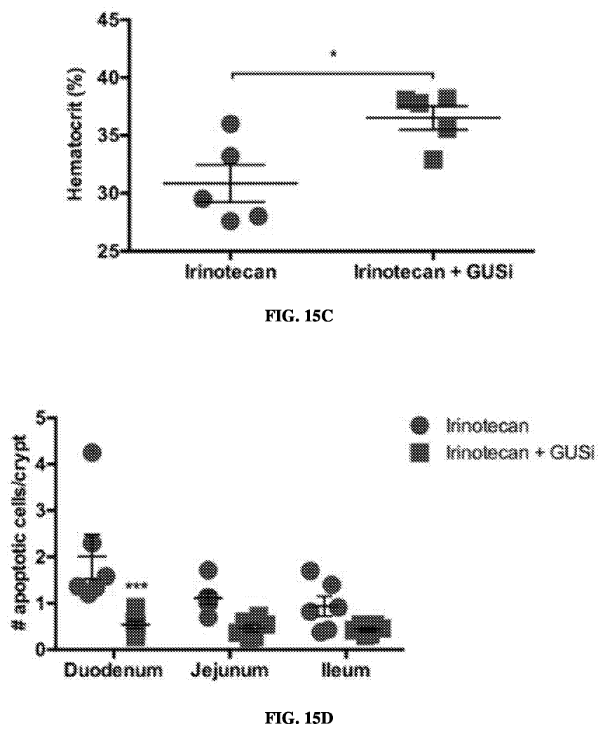

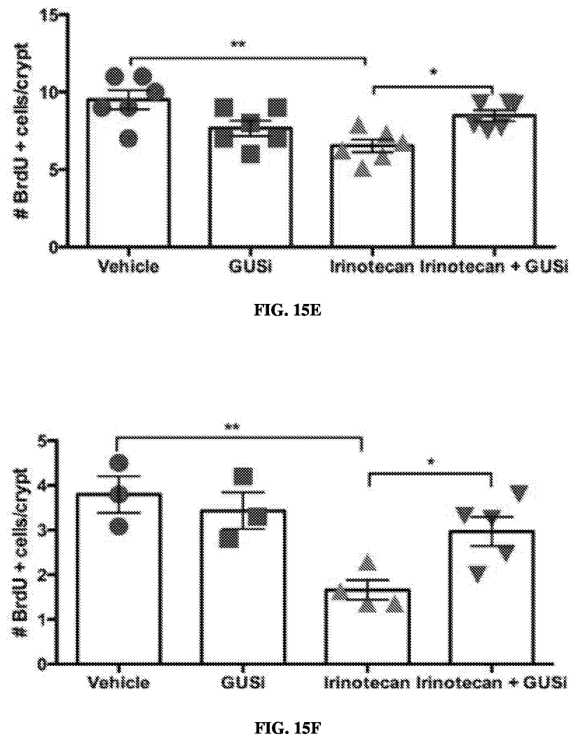

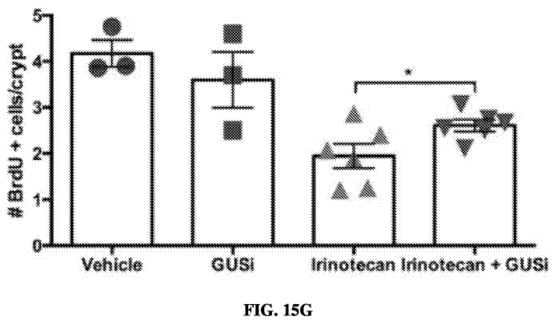

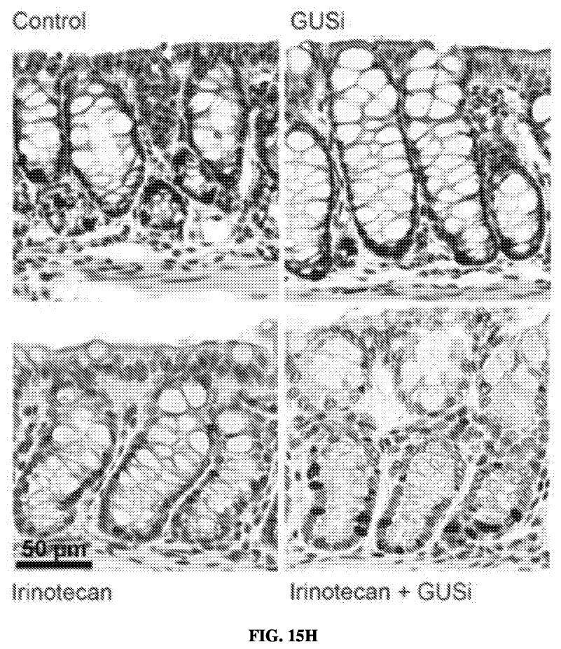

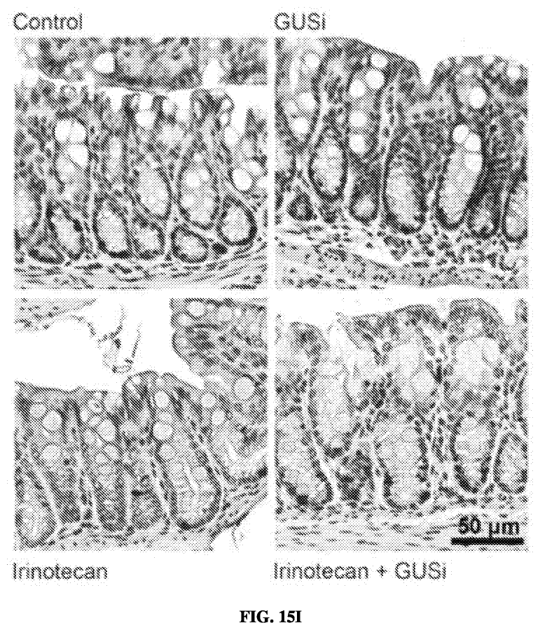

Five days (120 hours) after the single treatments outlined above, significant differences were observed with respect to the GI tract damage. First, at 120 hours, no changes between irinotecan alone and irinotecan plus Inh9 were observed in body weight or spleen mass (FIGS. 15A and 15B). In contrast to the 24-hour timepoint, though, a significant increase in hematocrit was observed in mice that also received Inh9 (FIG. 15C). Thus, at five days, Inh9 appears to protect this measure of animal health. Second, similar to the 24 timepoint, modest protection against increases in apoptotic cells per crypt were observed at 120 hours (FIG. 15D). Third, the levels of proliferative (BrdU+) cells per crypt were significantly decreased in the ilieum (FIG. 15E), proximal colon (FIG. 15F) and distal colon (FIG. 15G) of mice treated with irinotecan compared to vehicle or Inh9. These data indicate that irinotecan-induced intestinal damage take several days to appear in the GI tracts of treated animals. Fourth, co-treatment of irinotecan with Inh9 significantly increased the number of proliferative cells per crypt in the ilieum, proximal colon, and distal colon of mice after five days, as indicated by BrdU-positive staining (FIG. 15E-15G) and colon histology (FIGS. 15H and 15I). Thus, at 120 hours, a single Inh9 treatment is capable of significantly protecting the number of proliferative cells in the mouse GI tract five days after irinotecan dosing. This in vivo result is intriguing because, as outlined above, in fimo studies revealed that irinotecan and Inh9 impact gut microbial enzyme function at 24 hours, but not at 120 hours. As such, the protection afforded at 24 hours by Inh9 is sufficient to improve subsequent gut epithelial outcomes in mice several days after administration.

### Example 19

### GUS Inhibition Alleviates Irinotecan Toxicity in Tumor Xenograft Mice

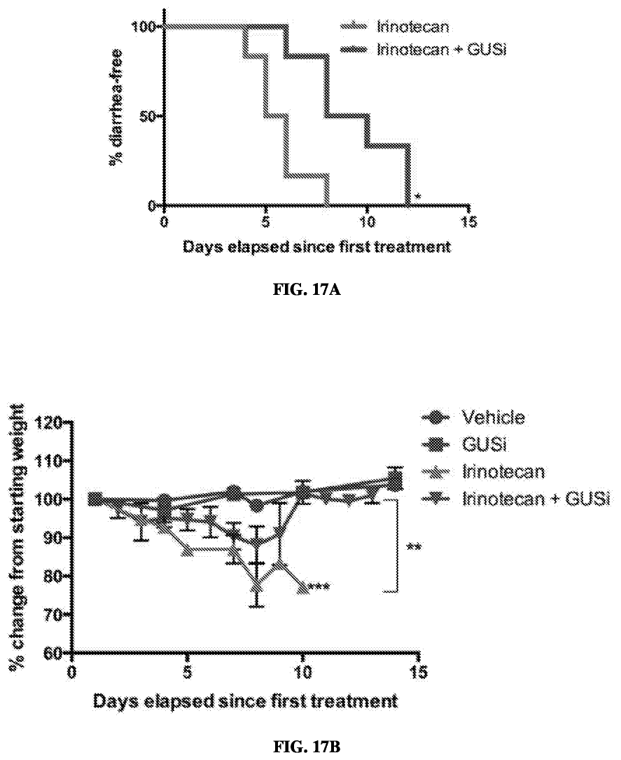

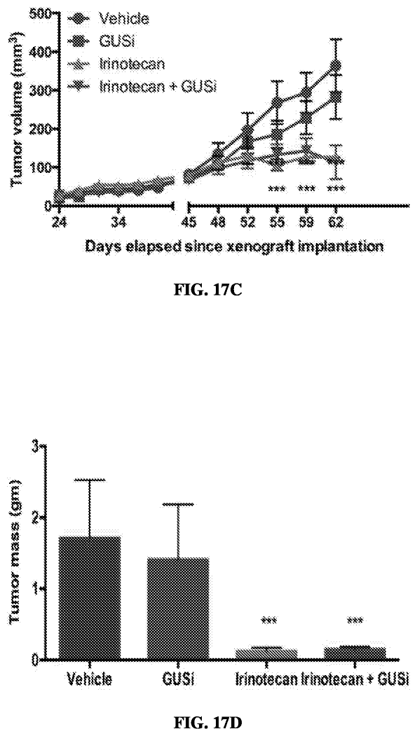

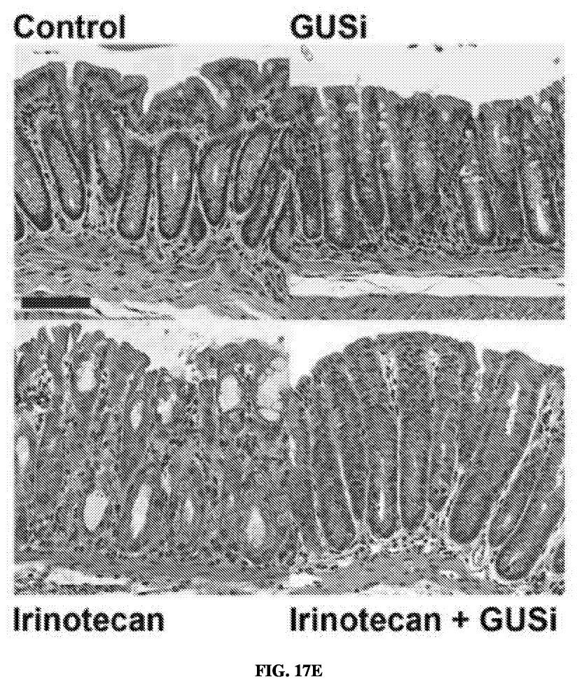

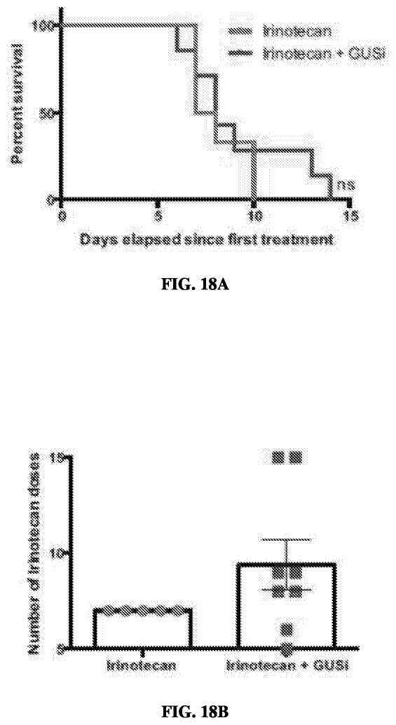

The in vitro and in fimo assays showed that Inh9 inhibited microbial GUS enzymes responsible for SN38-G reactivation. Thus, its effects were investigated on the antineoplastic efficacy of irinotecan in vivo. The well-described xenograft model of triple negative inflammatory breast cancer was used. SUM149 cells, derived from a primary inflammatory ductal carcinoma of the breast, are representative of triple negative breast cancer (ER−, PR−, Her2+) and readily form tumors when subcutaneously injected in immune-deficient mice. Once palpable tumors were detected, mice were randomized into four groups: control, Inh9 (1 mg/kg delivered as 10 μg twice daily p.o.), daily injections of 50 mg/kg irinotecan i.p. (IRI), and IRI+Inh9. Irinotecan induced early-onset diarrhea within 12 hours in both treatment groups, a phenomenon attributed to the cholinergic effects of the drug. However, 50% of mice receiving irinotecan alone developed late-onset, delayed diarrhea five days following the first treatment (FIG. 17A). Diarrhea was watery and, in many instances, perianal fur was tinged with blood. However, animals treated with both irinotecan and Inh9 showed significantly less diarrhea than those receiving irinotecan alone (FIG. 17A). Animals were euthanized when ≥20% reduction in body weight resulting from diarrhea was observed; 10 days following the first treatment, the entire irinotecan-treated cohort required euthanasia (FIG. 17B). In contrast, mice that received both irinotecan and Inh9 were significantly protected against irinotecan-induced weight loss (FIG. 17B). Furthermore, tumor-xenograft mice receiving irinotecan and Inh9 trended toward living longer than mice receiving irinotecan alone (FIG. 17B; FIG. 18A) but did not receive more doses of irinotecan than mice that did not receive Inh9 (FIG. 18B). These data indicate that Inh9 alleviates irinotecan-induced diarrhea and weight loss in mice bearing triple negative breast tumor xenografts.

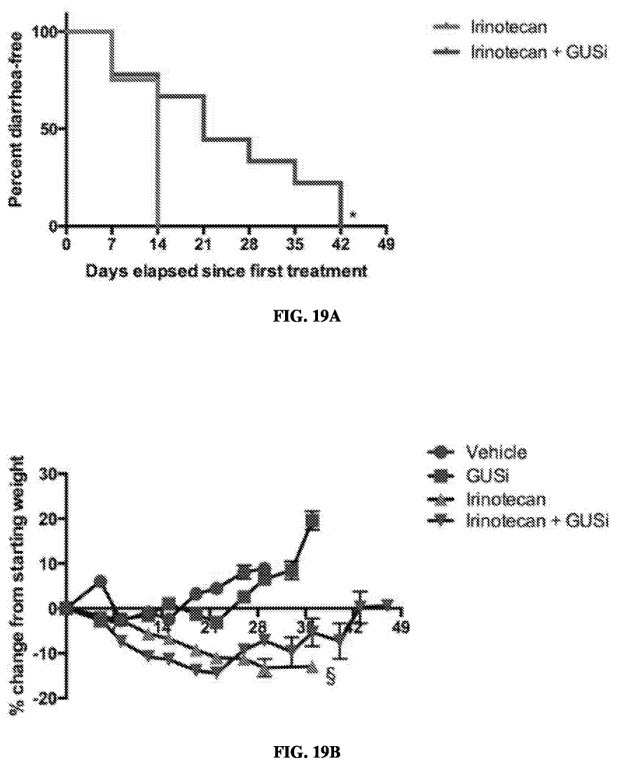

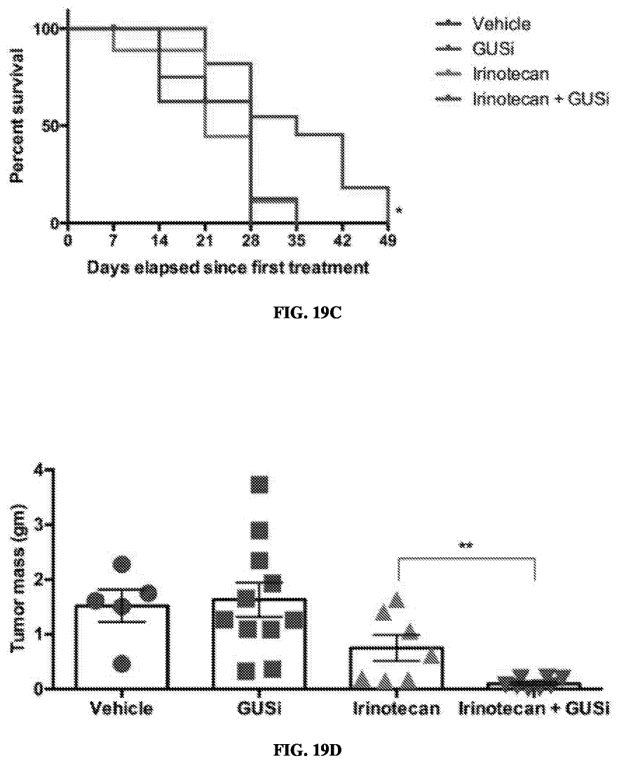

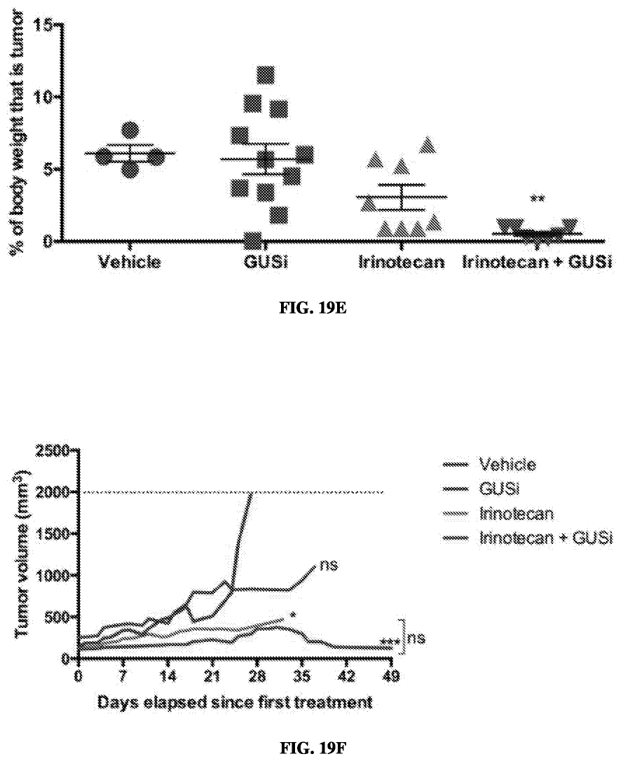

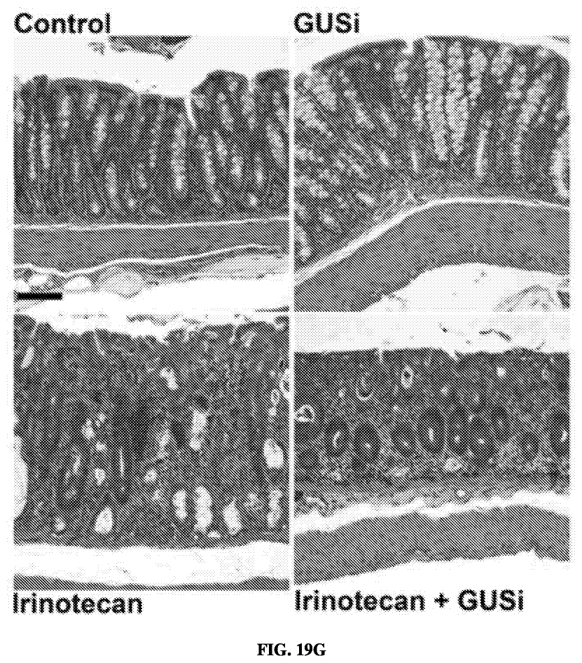

Tumor volumes were measured regularly throughout the experimental and revealed that both the irinotecan and irinotecan-plus-Inh9 cohorts displayed significantly reduced tumor volumes compared to the vehicle and Inh9 cohorts (FIG. 19C). Similarly, the terminal tumor masses measured upon animal sacrifice revealed that both irinotecan and irinotecan-plus-Inh9 animals showed significantly reduced tumors (FIG. 19D). Finally, post-sacrifice colon histological examination showed dramatic irinotecan-induced epithelial damage, and marked protection against this damage by Inh9 (FIG. 19E). Together, these results demonstrate that Inh9 alleviate irinotecan-induced diarrhea, weight loss and gut damage but does not impede the anti-tumor efficacy of irinotecan in a mouse tumor xenograft model of triple negative breast cancer.

### Example 20

### GUS Inhibition Dramatically Improves Irinotecan Efficacy in Tumor-Bearing GEMM Animals

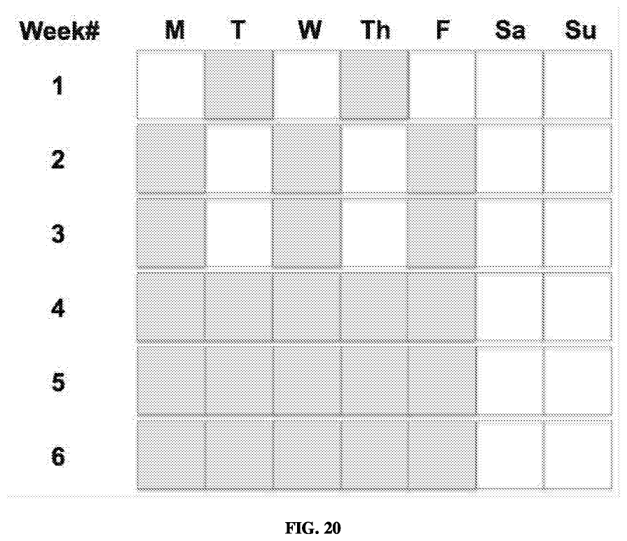

Also examined were irinotecan and GUS inhibitor in the C3-Tag genetically engineered mouse model (GEMM) of triple-negative breast cancer. Unlike the athymic xenograft mice above, these GEMM animals have an intact immune system. The 5′ flanking region of the C3(1) component of the rat prostatic steroid binding promoter drives the tissue-specific expression of the murine SV40 polyomavirus large T antigen, resulting in mammary tumors that histologically resemble human disease. Mice were maintained in specific pathogen-free housing and regularly monitored for mammary tumor development. Once palpable tumors were detected (˜100 mm³), mice were randomized into one of four groups: vehicle, Inh9, irinotecan, or irinotecan+Inh9. Irinotecan was provided at 50 mg/kg i.p. in a dose-intensification strategy, starting with two doses per week for one week, then three doses per week for two weeks, and finally five doses per week in all subsequent weeks (FIG. 20). Inh9 provided at 1 mg/kg per day p.o. (delivered as 10 μg twice daily) on irinotecan-do sing days. In this study, all animals treated with irinotecan alone developed diarrhea within 14 days, while animals treated with IRI+Inh9 experienced significantly reduced diarrhea, with some animals remaining diarrhea-free for 42 days (FIG. 19A). Thus, Inh9 effectively alleviates irinotecan-induced diarrhea in this breast cancer GEMM even under the dose-intensification regimen employed in this study.

Vehicle- and Inh9-treated control animals did not develop diarrhea, and were followed for 70-80 days until tumor size necessitated euthanasia (FIG. 19B). Due to tumor growth, vehicle- and Inh9-treated animals gained weight over the course of the study (FIG. 19B). Mice receiving irinotecan, though, consistently lost weight until euthanasia was required for all IRI animals 28 days after the initiation of therapy (FIG. 19B). In contrast, IRI+Inh9 animals lost more weight than the IRI treatment group in the first 21 days of the study, but then the IRI+Inh9 mice rebounded and recovered their lost weight and allowing the study to continue to day 49 (FIG. 19B). Indeed, Kaplan-Meier survival curves reveal that the IRI+Inh9 treatment lived significantly longer than each of the other treatment groups: 43% longer than IRI, and 29% longer than Veh and Inh9 (FIG. 19C). Thus, when used in conjunction with irinotecan, Inh9 significantly protects against animal weight loss and significantly improves animal survival in the C3Tag breast cancer GEMM model.

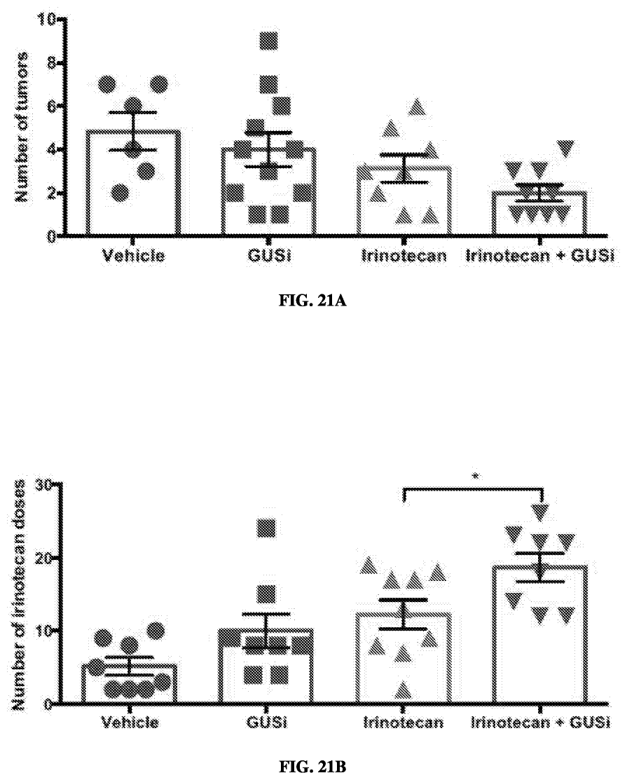

C3-Tag triple-negative breast cancer GEMM animals exhibit tumors at approximately eight weeks of age and, as stated above, when these tumors reached ˜100 mm³, treatment and control groups were established. Over the course of the study, the number of tumors observed in the animals in each treatment group did not differ, because neither IRI nor Inh9 would affect large T antigen-driven tumorigenesis in this GEMM (FIG. 21A). Upon sacrifice, the primary tumors in the vehicle and Inh9 groups averaged 1.5 g in mass and the accounted for more than 5% of the animals' body weight (FIGS. 19D and 19E). Mice receiving irinotecan alone showed trends toward smaller primary tumors (˜1 g) accounting for only 3% body weight, but these values were not statistically different than the vehicle and Inh9 groups (FIGS. 19D and 19E). By contrast, mice in the IRI+Inh9 group exhibited dramatically reduced primary tumor masses, uniformly less than 100 mg, that accounted for markedly less of the animals' body weight, below 1% (FIGS. 19D and 19E). Tumor volume measures collected throughout the course of the study also establish that irinotecan plus GUS inhibition produce significantly smaller tumors that allow the animals to live significantly longer than the irinotecan, vehicle or Inh9 groups (FIG. 19F). While Inh9 alone reduced tumor volumes as measured during the study (FIG. 19F), significant effects of Inh9 alone were not observed in primary tumor masses upon sacrifice (FIGS. 19D and 19E). These results demonstrate that GUS inhibition dramatically enhances the antitumor efficacy of irinotecan in this breast cancer GEMM.

Colon tissue examined by H&E staining revealed marked protection of irinotecan-induced gut damage by GUS inhibition. The vehicle and Inh9 treatment groups showed healthy colonic histology, with intact epithelial layers and well-formed glandular structure (FIG. 19G). By contrast, the irinotecan treatment group animals exhibited almost completely destroyed glandular structure, an infiltration of inflammatory cells, and a severely compromised epithelial layer (FIG. 19G). The IRI+Inh9 treatment animals, however, maintained a properly assembled colonic glandular structure, with reduced inflammatory infiltrates and a more intact epithelial layer (FIG. 19G). Indeed, in spite of the irinotecan dose-intensification regimen, mice receiving IRI+Inh9 maintained a healthier gut epithelium and an improved body weight; together, these factors allowed the IRI+Inh9 mice to receive more doses of irinotecan than the mice receiving IRI alone (FIG. 21B). Thus, Inh9 allows increased irinotecan dosing without the associated gut damage and other injuries, and facilitates dramatic reduction in tumors and increased survival.

### Example 21

### GUS Inhibitor Protects Against Gut Proteobacteria Expansion in Athymic Mice

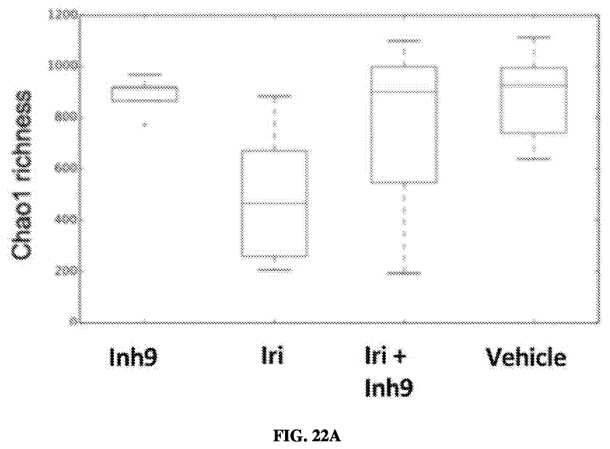

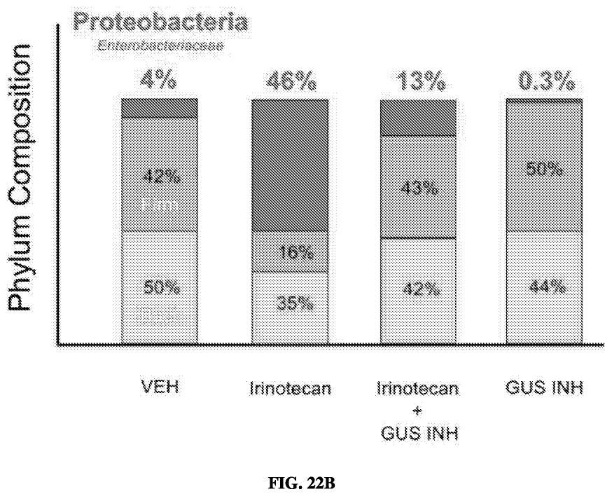

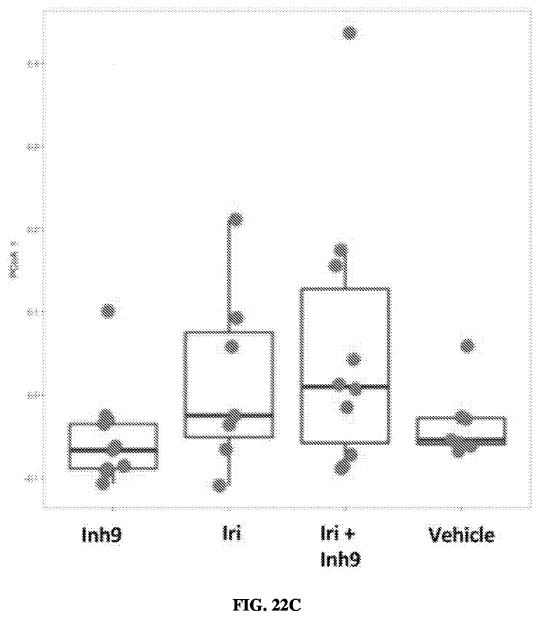

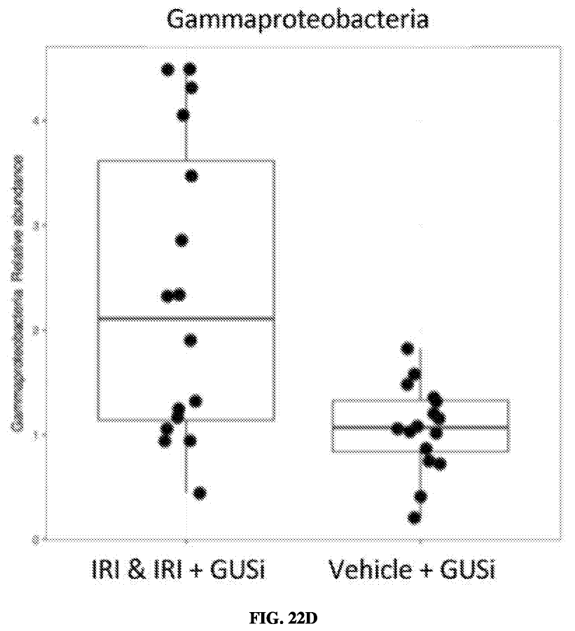

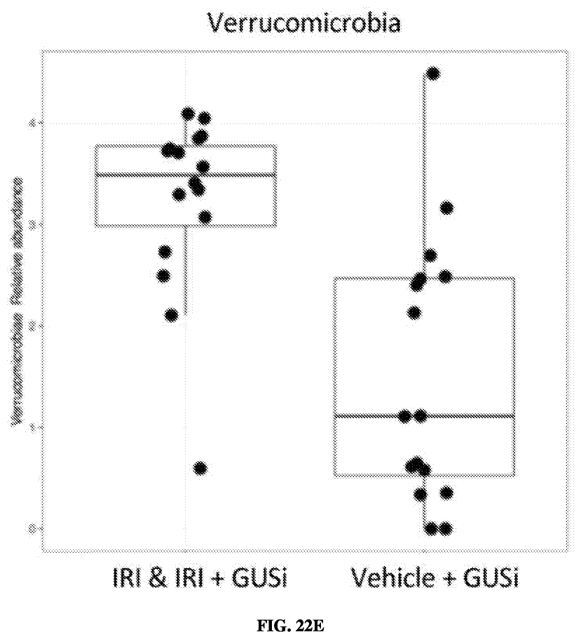

A metataxonomic analysis was performed of the luminal contents of athymic mice bearing Sum149 triple-negative breast cancer xenograft tumors by sequencing the V3-V4 region of the bacterial 16s rRNA gene. A significant drop was observed in species diversity, as assessed by Chao1 richness, in animals treated with irinotecan (FIG. 22A). Mice treated with IRI+Inh9, however, maintained species diversity to levels similar to that of vehicle controls (FIG. 22A). Inh9 treatment alone reduced variability in Chao1 between mice, and uniformly maximized species richness in that treatment group (FIG. 22A). Gut microbial diversity is associated with animal and human health, while reduced diversity is associated with disease in both model organisms and humans (REFs). Thus, Inh9 blunts the negative effects of irinotecan on gut microbial diversity in athymic tumor xenograft mice, and is capable of increases species richness in these animals.

Metataxonomic sequencing revealed that irinotecan produces a marked expansion of gut microbial Proteobacteria in athymic tumor xenograft mice. At the phylum level, the lumenal contents of vehicle-treated mice contained 50% Bacteroidetes, 42% Firmicutes, and 4% Proteobacteria (FIG. 22B). By contrast, irinotecan-treated mice exhibited a dramatic increase in the levels of Proteobacteria, up to 46%, and decreases in both Firmicutes (down to 16%) and Bacterodetes (35%) (FIG. 22B). In mice treated with both irinotecan and Inh9, however, the expansion of Proteobacteria was markedly reduced, down to only 13%, while maintaining near vehicle-treatment levels of Firmicutes (43%) and Bacteroidetes (42%) (FIG. 22B). Finally, we found that GUS inhibitor alone is able to reduce the levels of Proteobacteria in the mouse GI tract more than 10-fold, down to 0.3% compared to 4% observed in vehicle treated mice (FIG. 22B). Thus, in immune-deficient mice, Inh9 markedly protects gut microbial composition in athymic mice, increasing diversity and reducing irinotecan-induced increases in Proteobacteria.

Family-level taxonomic changes reveal that the Proteobacterial expansions observed with irinotecan arose from the Enterobacteriaceae, specifically the Salmonella, Escherichia, Shigella, Klebsiella and Yersinia (FIG. 22B). These opportunistic facultative aerobic pathogens can flourish in inflamed sites of epithelial damage that leak oxygen into the intestinal lumen. Indeed, the Enterobacteriaceae are the only intestinal taxa with a GUS operon containing the gus gene as well as glucuronide transporters. The operon is under the control of a glucuronide-sensing repressor, GusR, that up-regulates the expression of these glucuronide processing genes when a ligand containing glucuronic acid is detected (Little et al, 2018). This arrangement likely gives these relatively trace Enterobacteriaceae taxa an ability to utilize glucuronic acid as a carbon source, and to compete with the more abundant Firmicutes and Bacteroides in the gut. Thus, the increase in Enterobacteriaceae in irinotecan-treated mice may also be driven by SN-38-G's ability to provide a growth advantage to these microbial species, and that GUS inhibition blunts this outgrowth by blocking the ability of these bacterial from utilizing SN-38-G as a carbon source. In support of this theory, the Enterobacteriaceae contain only Loop 1 GUS enzymes, those that process SN-38-G best and are also most potently inhibited by GUS inhibitors, including Inh9. Together, these results in athymic mice may have important implications for patients with altered immune function.

### Example 22

### GUS Inhibition Does Not Alter Gut Microbial Composition in the Immune-Competent GEMM

Finally, we examined the effects of irinotecan and GUS inhibitor on the composition of the gut microbiota in C3Tag triple-negative breast cancer GEMM animals. In contrast to the results in the Sum149 triple-negative breast tumor xenografts in the immune-compromised athymic mice outlined above, we found that irinotecan was the sole driver of changes in microbiota composition in these GEMM mice with intact immune systems. While the vehicle and Inh9 treatment groups appeared similar by PCoA1 analysis, both the irinotecan and IRI+Inh9 groups were similar to each other and distinct from the non-irinotecan mice (FIG. 22C). However, unlike the xenograft mice, irinotecan induces much smaller changes in the composition of the gut microbiota in these GEMM animals. At the phylum and class levels, we found that irinotecan, either alone or in combination with GUS inhibitor, lead to significant increases Proteobacteria and Gammaproteobacteria, as well as Verrucomicrobia and Verrocumicrobiae compared to groups receiving no irinotecan (FIG. 22D). These changes were not as dramatic as those observed with the athymic Sum149 xenograft mice presented above. They indicate that GUS inhibition does not prevent even modest increases bacteria of the Gammaproteobacteria and Verrucomicrobiae, which include E. coli and Akkermansia mucinophila, respectively, in mice treated with irinotecan. In these GEMM animals, though, no change in Enterobacteriaceae or other Gammaproteobacteria families was observed; thus, the taxa that drive the increases in Proteobacteria are not known in these immune-competent mice. The increase in Verrucomicrobiae is associated with a significant increase in Akkermansia mucinophila, a microbe typically associated with a healthy gut and the processing of host mucins. Taken together, these results indicate that, in mice with an intact immune system, GUS inhibition does not change the composition of the gut microbiota.

All technical and scientific terms used herein have the same meaning. Efforts have been made to ensure accuracy with respect to numbers used (e.g. amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

### BIBLIOGRAPHY

1. T. Conroy, F. Desseigne, M. Ychou, O. Bouche, R. Guimbaud, Y. Becouarn, A. Adenis, J. L. Raoul, S. Gourgou-Bourgade, C. de la Fouchardiere, J. Bennouna, J. B. Bachet, F. Khemissa-Akouz, D. Pere-Verge, C. Delbaldo, E. Assenat, B. Chauffert, P. Michel, C. Montoto-Grillot, M. Ducreux, U. Groupe Tumeurs Digestives of, P. Intergroup, FOLFIRINOX versus gemcitabine for metastatic pancreatic cancer. N Engl J Med 364, 1817-1825 (2011).
2. Y. Pommier, Topoisomerase I inhibitors: camptothecins and beyond. Nat Rev Cancer 6, 789-802 (2006).
3. P. F. Peddi, S. Lubner, R. McWilliams, B. R. Tan, J. Picus, S. M. Sorscher, R. Suresh, A. C. Lockhart, J. Wang, C. Menias, F. Gao, D. Linehan, A. Wang-Gillam, Multi-institutional experience with FOLFIRINOX in pancreatic adenocarcinoma. JOP 13, 497-501 (2012).
4. N. Peekhaus, T. Conway, What's for dinner?: Entner-Doudoroff metabolism in Escherichia coli. J Bacteriol 180, 3495-3502 (1998).
5. K. Takasuna, T. Hagiwara, M. Hirohashi, M. Kato, M. Nomura, E. Nagai, T. Yokoi, T. Kamataki, Involvement of beta-glucuronidase in intestinal microflora in the intestinal toxicity of the antitumor camptothecin derivative irinotecan hydrochloride (CPT-11) in rats. Cancer Res 56, 3752-3757 (1996).
6. S. H. Pedroso, A. T. Vieira, R. W. Bastos, J. S. Oliveira, C. T. Cartelle, R. M. Arantes, P. M. Soares, S. V. Generoso, V. N. Cardoso, M. M. Teixeira, J. R. Nicoli, F. S. Martins, Evaluation of mucositis induced by irinotecan after microbial colonization in germ-free mice. Microbiology 161, 1950-1960 (2015).
7. A. M. Stringer, R. J. Gibson, J. M. Bowen, R. M. Logan, K. Ashton, A. S. Yeoh, N. Al-Dasooqi, D. M. Keefe, Irinotecan-induced mucositis manifesting as diarrhoea corresponds with an amended intestinal flora and mucin profile. Int J Exp Pathol 90, 489-499 (2009).
8. X. B. Lin, L. A. Dieleman, A. Ketabi, I. Bibova, M. B. Sawyer, H. Xue, C. J. Field, V. E. Baracos, M. G. Ganzle, Irinotecan (CPT-11) chemotherapy alters intestinal microbiota in tumour bearing rats. PLoS One 7, e39764 (2012).
9. R. A. Forsgard, V. G. Marrachelli, K. Korpela, R. Frias, M. C. Collado, R. Korpela, D. Monleon, T. Spillmann, P. Osterlund, Chemotherapy-induced gastrointestinal toxicity is associated with changes in serum and urine metabolome and fecal microbiota in male Sprague-Dawley rats. Cancer Chemother Pharmacol 80, 317-332 (2017).
10. M. J. Blaser, The theory of disappearing microbiota and the epidemics of chronic diseases. Nat Rev Immunol 17, 461-463 (2017).
11. S. P. Rosshart, B. G. Vassallo, D. Angeletti, D. S. Hutchinson, A. P. Morgan, K. Takeda, H. D. Hickman, J. A. McCulloch, J. H. Badger, N. J. Ajami, G. Trinchieri, F. Pardo-Manuel de Villena, J. W. Yewdell, B. Rehermann, Wild Mouse Gut Microbiota Promotes Host Fitness and Improves Disease Resistance. Cell, (2017).
12. J. C. Arthur, R. Z. Gharaibeh, J. M. Uronis, E. Perez-Chanona, W. Sha, S. Tomkovich, M. Muhlbauer, A. A. Fodor, C. Jobin, VSL #3 probiotic modifies mucosal microbial composition but does not reduce colitis-associated colorectal cancer. Sci Rep 3, 2868 (2013).
13. R. S. Kootte, E. Levin, J. Salojarvi, L. P. Smits, A. V. Hartstra, S. D. Udayappan, G. Hermes, K. E. Bouter, A. M. Koopen, J. J. Holst, F. K. Knop, E. E. Blaak, J. Zhao, H. Smidt, A. C. Harms, T. Hankemeijer, J. Bergman, H. A. Romijn, F. G. Schaap, S. W. M. Olde Damink, M. T. Ackermans, G. M. Dallinga-Thie, E. Zoetendal, W. M. de Vos, M. J. Serlie, E. S. G. Stroes, A. K. Groen, M. Nieuwdorp, Improvement of Insulin Sensitivity after Lean Donor Feces in Metabolic Syndrome Is Driven by Baseline Intestinal Microbiota Composition. Cell Metab 26, 611-619 e616 (2017).
14. T. R. Abrahamsson, H. E. Jakobsson, A. F. Andersson, B. Bjorksten, L. Engstrand, M. C. Jenmalm, Low gut microbiota diversity in early infancy precedes asthma at school age. Clin Exp Allergy 44, 842-850 (2014).
15. R. Kong, T. Liu, X. Zhu, S. Ahmad, A. L. Williams, A. T. Phan, H. Zhao, J. E. Scott, L. A. Yeh, S. T. Wong, Old drug new use—amoxapine and its metabolites as potent bacterial beta-glucuronidase inhibitors for alleviating cancer drug toxicity. Clin Cancer Res 20, 3521-3530 (2014).
16. R. M. Pollet, E. H. D'Agostino, W. G. Walton, Y. Xu, M. S. Little, K. A. Biernat, S. J. Pellock, L. M. Patterson, B. C. Creekmore, H. N. Isenberg, R. R. Bahethi, A. P. Bhatt, J. Liu, R. Z. Gharaibeh, M. R. Redinbo, An Atlas of beta-Glucuronidases in the Human Intestinal Microbiome. Structure 25, 967-977 e965 (2017).
17. W. Yang, B. Wei, R. Yan, Amoxapine Demonstrates Incomplete Inhibition of beta-Glucuronidase Activity from Human Gut Microbiota. SLAS Discov, 2472555217725264 (2017).
18. B. D. Wallace, H. Wang, K. T. Lane, J. E. Scott, J. Orans, J. S. Koo, M. Venkatesh, C. Jobin, L. A. Yeh, S. Mani, M. R. Redinbo, Alleviating cancer drug toxicity by inhibiting a bacterial enzyme. Science 330, 831-835 (2010).
19. A. B. Roberts, B. D. Wallace, M. K. Venkatesh, S. Mani, M. R. Redinbo, Molecular insights into microbial beta-glucuronidase inhibition to abrogate CPT-11 toxicity. Mol Pharmacol 84, 208-217 (2013).
20. B. D. Wallace, A. B. Roberts, R. M. Pollet, J. D. Ingle, K. A. Biernat, S. J. Pellock, M. K. Venkatesh, L. Guthrie, S. K. O'Neal, S. J. Robinson, M. Dollinger, E. Figueroa, S. R. McShane, R. D. Cohen, J. Jin, S. V. Frye, W. C. Zamboni, C. Pepe-Ranney, S. Mani, L. Kelly, M. R. Redinbo, Structure and Inhibition of Microbiome beta-Glucuronidases Essential to the Alleviation of Cancer Drug Toxicity. Chem Biol 22, 1238-1249 (2015).
21. A. P. Bhatt, M. R. Redinbo, S. J. Bultman, The role of the microbiome in cancer development and therapy. CA Cancer J Clin 67, 326-344 (2017).

That which is claimed:

1. A method for treating a condition or enhancing a chemotherapeutic regimen, comprising administering a compound of Formula I: 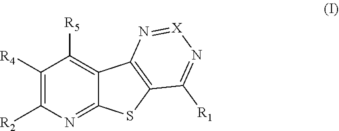
wherein,
R₁ is selected from the group consisting of:
i. —OR_{A}, wherein R_{A} is selected from the group consisting of H, CF₃, optionally substituted linear or branched C₂₋₆ alkyl, optionally substituted benzyl, and C₃₋₈ cycloalkyl;
ii. —NR_{B}R_{C}, wherein R_{B} and R_{C} are each independently selected from the group consisting of H, linear or branched C₁₋₆ alkyl optionally substituted with amino, optionally substituted benzyl, C₂₋₅ heteroaryl, C₂₋₅ heteroalkyl, and C₃₋₈ cycloalkyl;
iii. C₂₋₅ heteroaryl optionally substituted with hydroxyl, halo, or amino;
iv. —N(CH₂)ₚ—Z, wherein p is 1 or 2; and Z is heteroaryl or heterocycloalkyl optionally substituted with hydroxyl, halo, or amino;
v. 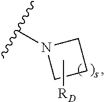
wherein s is 1, 2, or 3; and R_{D} is amino, hydroxyl, halo, or linear or branched C₁₋₆ alkyl; and
vi. 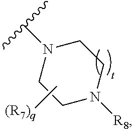
wherein
R₇ is H, linear or branched C₁₋₆ alkyl optionally substituted with halo, amino, or hydroxyl;
t is 1 or 2;
q is 1 or 2, wherein R₇ can join form a bridge; and
R₈ is H, linear or branched C₁₋₄ alkyl optionally substituted with halo, hydroxyl, or amino;
R₂ is H, aryl, heterocycloalkyl, or —NR_{F}R_{G}, wherein
R_{F} and R_{G} are independently selected from the group consisting of H, linear or branched C₁₋₆ alkyl optionally substituted with halo, amino or hydroxyl, optionally substituted benzyl, and C₃₋₈ cycloalkyl;
X is N or —CR₃, wherein
R₃ is selected from the group consisting of H, halogen, —OR_{I}, and —NR_{I}R_{J}, wherein
R_{I} and R_{J} are each independently selected from H and linear or branched C₁₋₆ alkyl, wherein said alkyl can be optionally substituted with hydroxyl; and
R₄ and R₅ are H or are taken together with the carbon to which each is attached to form an optionally substituted C₅₋₇ membered ring.

2. The method of claim 1, wherein R₄ and R₅ are taken together with the carbon to which each is attached to form an optionally substituted C₅₋₇ membered ring.

3. The method of claim 2, wherein X is N.

4. The method of claim 3, wherein R₂ is heterocycloalkyl.

5. The method of claim 4, wherein R₂ is a 4-8 membered ring containing 1 or 2 nitrogen atoms.

6. The method of claim 5, wherein R₂ is chosen from the group consisting of:

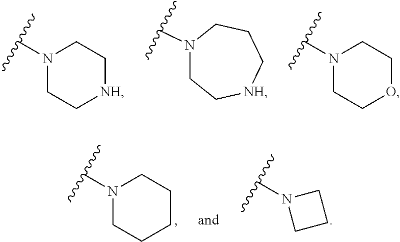

7. The method of claim 6, wherein R₂ is morpholinyl.

8. The method of claim 7, wherein R₁ is
—NR_{B}R_{C}, wherein R_{B} and R_{C} are each independently selected from the group consisting of H, linear or branched C₁₋₆ alkyl optionally substituted with amino, optionally substituted benzyl, C₂₋₅ heteroaryl, C₂₋₅ heteroalkyl, and C₃₋₈ cycloalkyl;
R₁ is C₂₋₅ heteroaryl optionally substituted with hydroxyl, halo, or amino; or
R₁ is 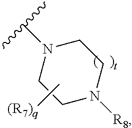
wherein
R₇ is H, linear or branched C₁₋₆ alkyl optionally substituted with halo, amino, or hydroxyl;
t is 1 or 2;
q is 1 or 2 and when q is 2 the two R₇ groups may join to form a bridged compound; and
R₈ is H, linear or branched C₁₋₄ alkyl optionally substituted with halo, hydroxyl, or amino.

9. The method of claim 8, wherein R₁ is 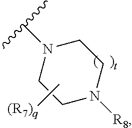
wherein R₇ is H, linear or branched C₁₋₆ alkyl optionally substituted with halo, amino, or hydroxyl;
t is 1 or 2;
q is 1 or 2, wherein R₇ can join form a bridge; and
R₈ is H, linear or branched C₁₋₄ alkyl optionally substituted with halo, hydroxyl, or amino.

10. The method of claim 9, wherein t and q are each 1.

11. The method of claim 10, wherein R₈ is H.

12. The method of claim 11, wherein R₇ is methyl.

13. The method of claim 3, wherein R₁ is 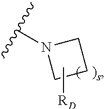
wherein
s is 1, 2, or 3; and
R_{D} is amino, hydroxyl, halo, or linear or branched C₁₋₆ alkyl;
or
R₁ is 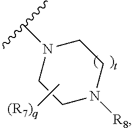
wherein
R₇ is H, linear or branched C₁₋₆ alkyl optionally substituted with halo, amino, or hydroxyl;
t is 1 or 2;
q is 1 or 2, wherein R₇ can join form a bridge; and
R₈ is H, linear or branched C₁₋₄ alkyl optionally substituted with halo, hydroxyl, or amino.

14. The method of claim 13, wherein R₁ is 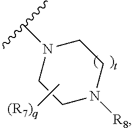
wherein
R₇ is H, linear or branched C₁₋₆ alkyl optionally substituted with halo, amino, or hydroxyl;
t is 1 or 2;
q is 1 or 2, wherein R₇ can join form a bridge; and
R₈ is H, linear or branched C₁₋₄ alkyl optionally substituted with halo, hydroxyl, or amino.

15. The method of claim 14, wherein t and q are each 1.

16. The method of claim 15, wherein R₇ is H.

17. The method of claim 16, wherein R₈ is H.

18. The method of claim 17, wherein R₂ is H, heterocycloalkyl, or —NR_{F}R_{G}, wherein
R_{F} and R_{G} are independently selected from the group consisting of H, linear or branched C₁₋₆ alkyl optionally substituted with halo, amino or hydroxyl, optionally substituted benzyl, and C₃₋₈ cycloalkyl.

19. The method of claim 18, wherein R₂ is heterocycloalkyl.

20. The method of claim 19, wherein the compound of Formula I is:

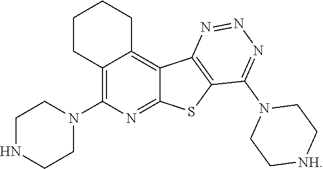 [SMILES: C1CCc2c(c(N3CCNCC3)nc3sc4c(N5CCNCC5)nnnc4c23)C1]

21. The method of claim 19, wherein the compound of Formula I is:

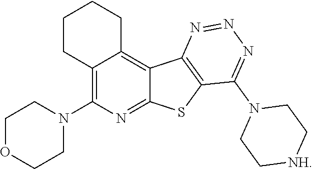 [SMILES: C1CCc2c(c(N3CCOCC3)nc3sc4c(N5CCNCC5)nnnc4c23)C1]

22. The method of claim 18, wherein R₂ is —NR_{F}R_{G}, wherein
R_{F} and R_{G} are independently selected from the group consisting of H, linear or branched C₁₋₆ alkyl optionally substituted with halo, amino or hydroxyl, optionally substituted benzyl, and C₃₋₈ cycloalkyl.

23. The method of claim 22, wherein R_{F} and R_{G} are independently selected from H or linear or branched C₁₋₆ alkyl optionally substituted with halo, amino or hydroxyl.

24. The method of claim 23, wherein the compound of Formula I is

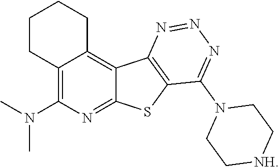 [SMILES: CN(C)c1nc2sc3c(N4CCNCC4)nnnc3c2c2c1CCCC2]

25. The method of claim 23, wherein the compound of Formula I is

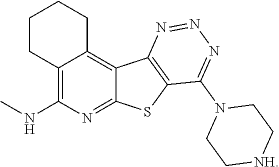 [SMILES: CNc1nc2sc3c(N4CCNCC4)nnnc3c2c2c1CCCC2]

26. The method of claim 1, wherein R₄ and R₅ are H.

27. The method of claim 26, wherein R₁ is 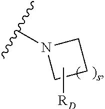
wherein
s is 1, 2, or 3; and
R_{D} is amino, hydroxyl, halo, or linear or branched C₁₋₆ alkyl;
or
R₁ is 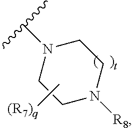
wherein
R₇ is H, linear or branched C₁₋₆ alkyl optionally substituted with halo, amino, or hydroxyl;
t is 1 or 2;
q is 1 or 2, wherein R₇ can join form a bridge; and
R₈ is H, linear or branched C₁₋₄ alkyl optionally substituted with halo, hydroxyl, or amino.

28. The method of claim 27, wherein R₁ is 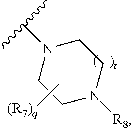
wherein
R₇ is H, linear or branched C₁₋₆ alkyl optionally substituted with halo, amino, or hydroxyl;
t is 1 or 2;
q is 1 or 2, wherein R₇ can join form a bridge; and
R₈ is H, linear or branched C₁₋₄ alkyl optionally substituted with halo, hydroxyl, or amino.

29. The method of claim 28, wherein t and q are each 1.

30. The method of claim 29, wherein R₇ is H.

31. The method of claim 30, wherein R₁ is piperazinyl.

32. The method of claim 31, wherein R₂ is H, aryl, heterocycloalkyl, or —NR_{F}R_{G},
wherein
R_{F} and R_{G} are independently selected from the group consisting of H, linear or branched C₁₋₆ alkyl, optionally substituted benzyl, and C₃₋₈ cycloalkyl.

33. The method of claim 32, wherein R₂ is aryl.

34. The method of claim 33, wherein R₂ is phenyl.

35. The method of claim 32, wherein R₂ is heterocycloalkyl.

36. The method of claim 35, wherein R₂ is a 4-8 membered ring containing 1 or 2 nitrogen atoms contained within the ring.

37. The method of claim 36, wherein R₂ is chosen from the group consisting of:

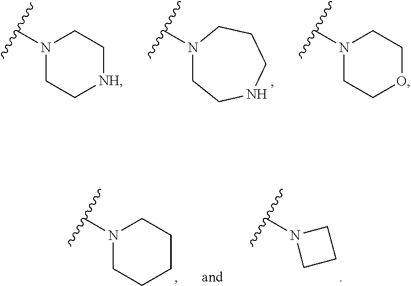 [SMILES: *N1CCNCC1 ; *N1CCCNCC1 ; *N1CCOCC1 ; *N1CCCCC1 ; *N1CCC1]

38. The method of claim 37, wherein R₂ is morpholinyl.

39. The method of claim 32, wherein R₂ is —NR_{F}R_{G}, wherein
R_{F} and R_{G} are independently selected from the group consisting of H, linear or branched C₁₋₆ alkyl optionally substituted with halo, amino or hydroxyl, optionally substituted benzyl, and C₃₋₈ cycloalkyl.

40. The method of claim 39, wherein R_{F} and R_{G} are independently H or linear or branched C₁₋₆ alkyl optionally substituted with halo, amino or hydroxyl.

41. The method of claim 40, wherein R_{F} and R_{G} are independently H or unsubstituted linear C₁₋₆ alkyl.

42. The method of claim 41, wherein the compound of Formula I is

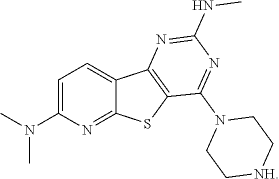 [SMILES: CNc1nc(N2CCNCC2)c2sc3nc(N(C)C)ccc3c2n1]

43. The method of claim 42, wherein the compound of Formula I is

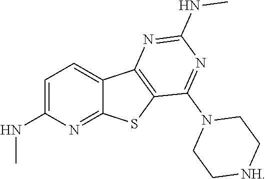 [SMILES: CNc1ccc2c(n1)sc1c(N3CCNCC3)nc(NC)nc12]

44. The method of claim 1, wherein the compound of Formula I has one of the following structures:

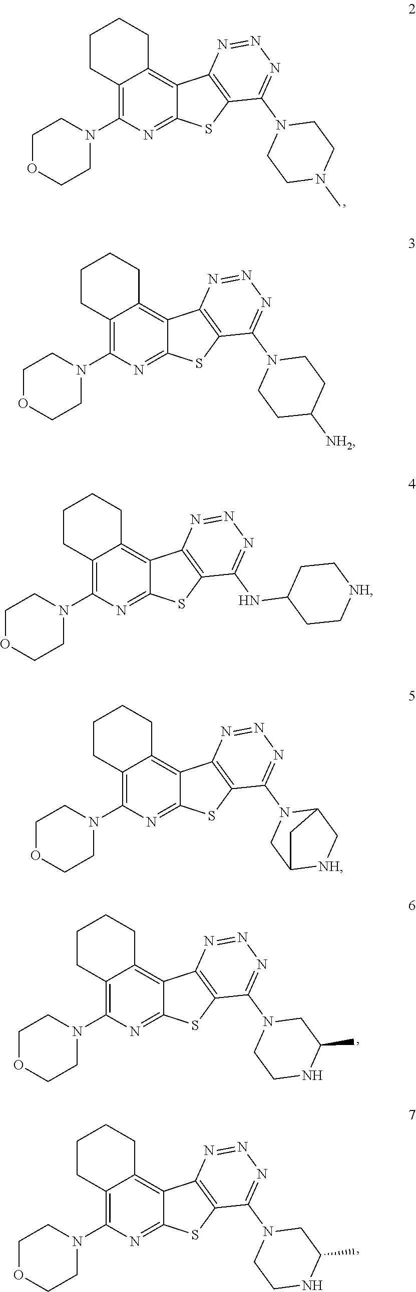 [SMILES: CN1CCN(c2nnnc3c2sc2nc(N4CCOCC4)c4c(c23)CCCC4)CC1 ; NC1CCN(c2nnnc3c2sc2nc(N4CCOCC4)c4c(c23)CCCC4)CC1 ; C1CCc2c(c(N3CCOCC3)nc3sc4c(NC5CCNCC5)nnnc4c23)C1 ; C1CCc2c(c(N3CCOCC3)nc3sc4c(N5CC6CC5CN6)nnnc4c23)C1 ; C[C@@H]1CN(c2nnnc3c2sc2nc(N4CCOCC4)c4c(c23)CCCC4)CCN1 ; C[C@H]1CN(c2nnnc3c2sc2nc(N4CCOCC4)c4c(c23)CCCC4)CCN1]

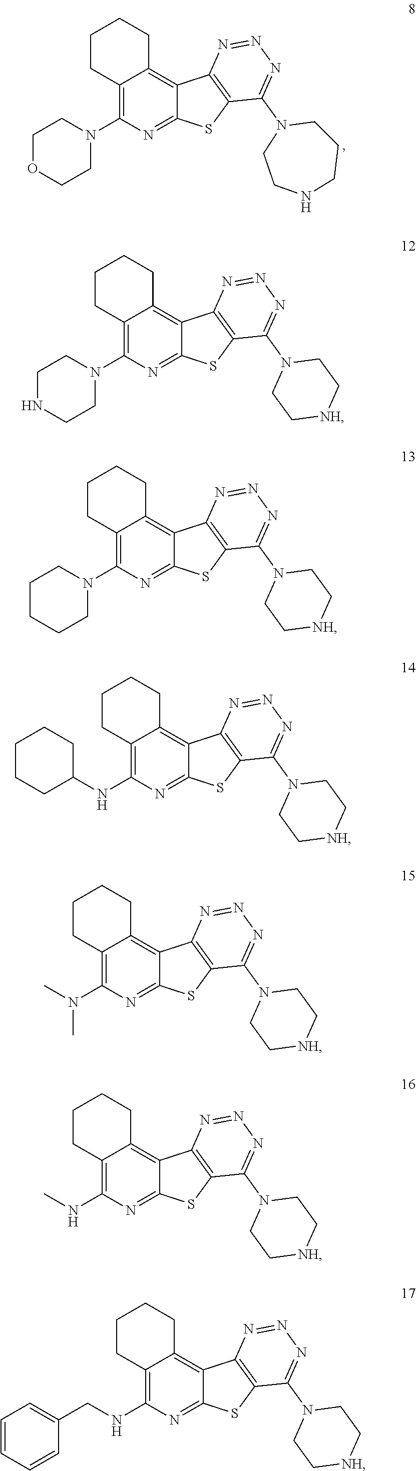

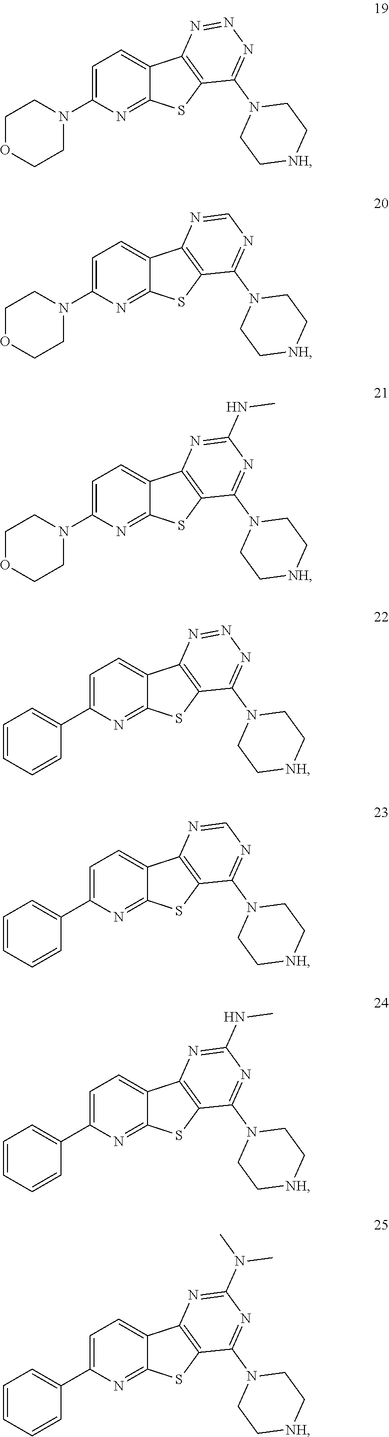

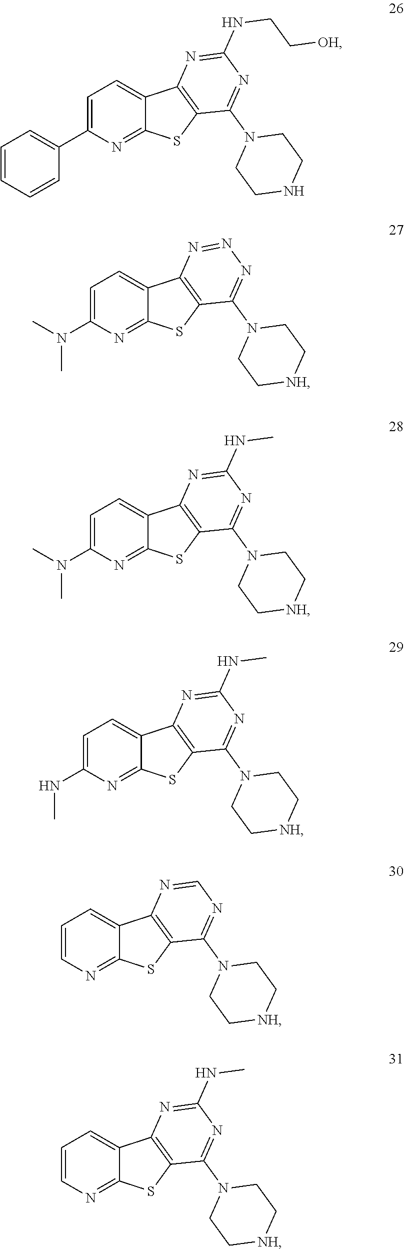

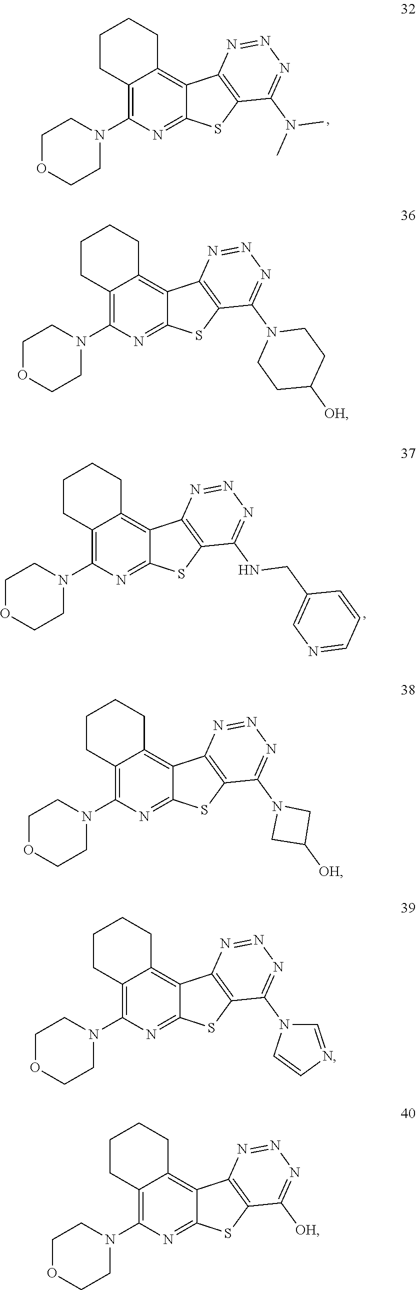 [SMILES: CN(C)c1nnnc2c1sc1nc(N3CCOCC3)c3c(c12)CCCC3 ; OC1CCN(c2nnnc3c2sc2nc(N4CCOCC4)c4c(c23)CCCC4)CC1 ; c1cncc(CNc2nnnc3c2sc2nc(N4CCOCC4)c4c(c23)CCCC4)c1 ; OC1CN(c2nnnc3c2sc2nc(N4CCOCC4)c4c(c23)CCCC4)C1 ; c1cn(-c2nnnc3c2sc2nc(N4CCOCC4)c4c(c23)CCCC4)cn1 ; Oc1nnnc2c1sc1nc(N3CCOCC3)c3c(c12)CCCC3]

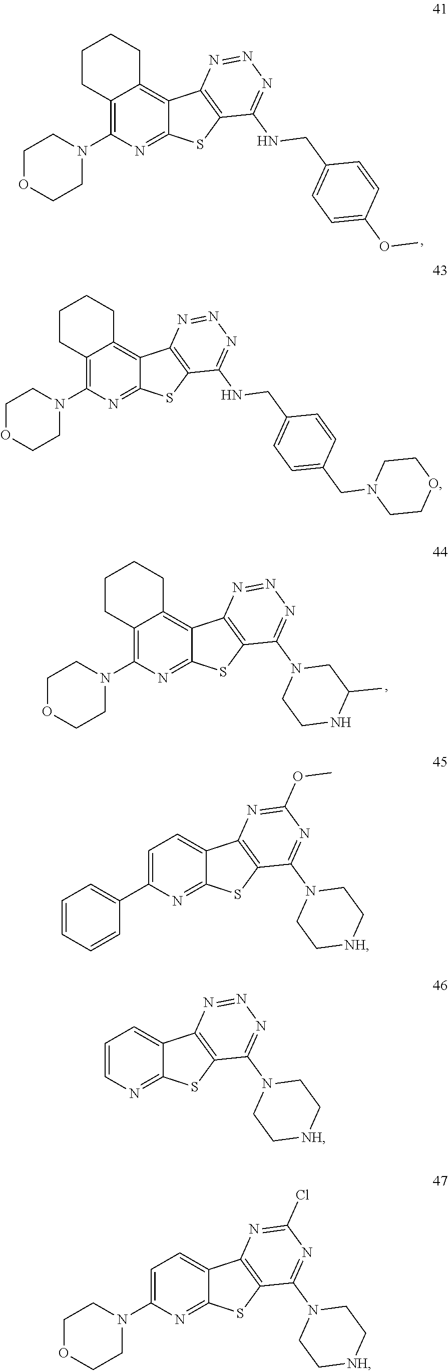 [SMILES: COc1ccc(CNc2nnnc3c2sc2nc(N4CCOCC4)c4c(c23)CCCC4)cc1 ; c1cc(CN2CCOCC2)ccc1CNc1nnnc2c1sc1nc(N3CCOCC3)c3c(c12)CCCC3 ; CC1CN(c2nnnc3c2sc2nc(N4CCOCC4)c4c(c23)CCCC4)CCN1 ; COc1nc(N2CCNCC2)c2sc3nc(-c4ccccc4)ccc3c2n1 ; c1cnc2sc3c(N4CCNCC4)nnnc3c2c1 ; Clc1nc(N2CCNCC2)c2sc3nc(N4CCOCC4)ccc3c2n1]

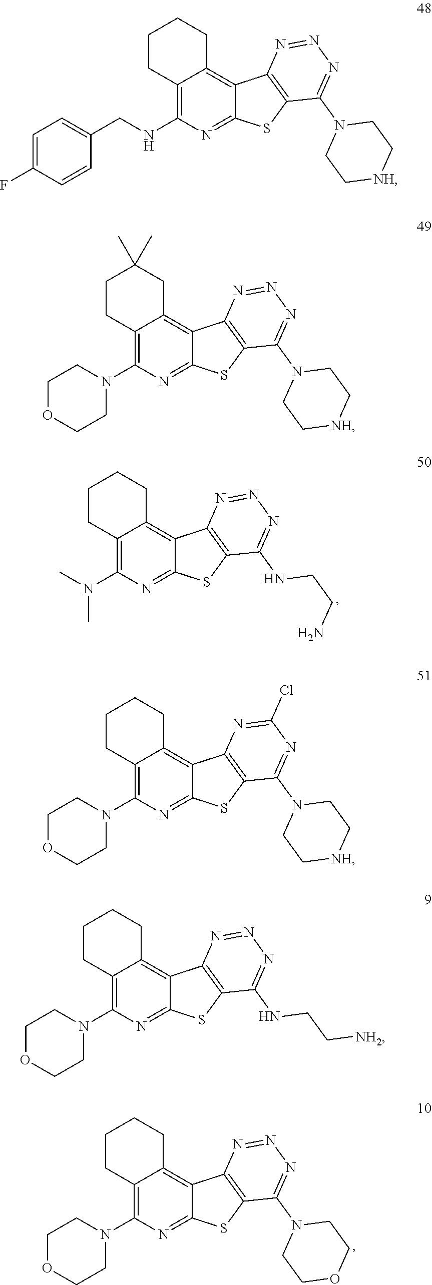 [SMILES: Fc1ccc(CNc2nc3sc4c(N5CCNCC5)nnnc4c3c3c2CCCC3)cc1 ; CC1(C)CCc2c(N3CCOCC3)nc3sc4c(N5CCNCC5)nnnc4c3c2C1 ; CN(C)c1nc2sc3c(NCCN)nnnc3c2c2c1CCCC2 ; Clc1nc(N2CCNCC2)c2sc3nc(N4CCOCC4)c4c(c3c2n1)CCCC4 ; NCCNc1nnnc2c1sc1nc(N3CCOCC3)c3c(c12)CCCC3 ; C1CCc2c(c(N3CCOCC3)nc3sc4c(N5CCOCC5)nnnc4c23)C1]

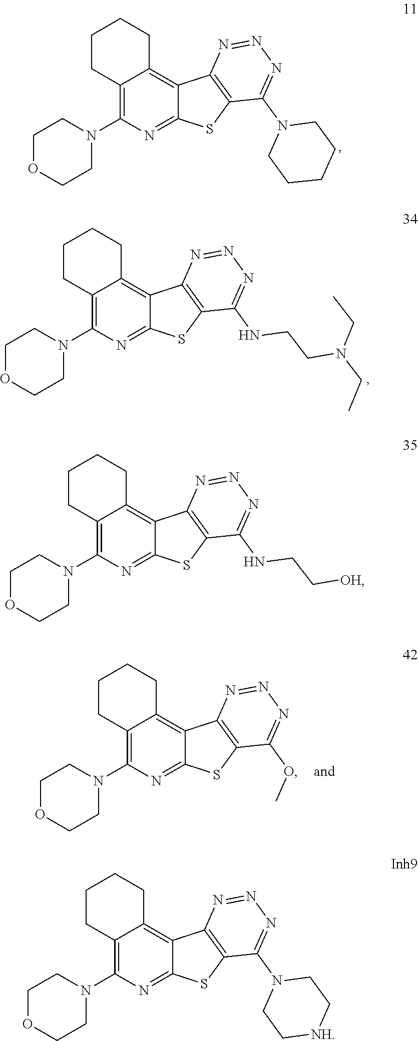 [SMILES: C1CCN(c2nnnc3c2sc2nc(N4CCOCC4)c4c(c23)CCCC4)CC1 ; CCN(CC)CCNc1nnnc2c1sc1nc(N3CCOCC3)c3c(c12)CCCC3 ; OCCNc1nnnc2c1sc1nc(N3CCOCC3)c3c(c12)CCCC3 ; COc1nnnc2c1sc1nc(N3CCOCC3)c3c(c12)CCCC3 ; C1CCc2c(c(N3CCOCC3)nc3sc4c(N5CCNCC5)nnnc4c23)C1]

45. The method of claim 1, for treating a condition, wherein said condition is chemotherapy induced loss of body weight.

46. The method of claim 45, wherein said loss of body weight is reduced from about 5% to about 30%.

47. The method of claim 45, wherein said loss of body weight is reduced from about 10% to about 20%.

48. The method of claim 45, wherein said loss of body weight is delayed by about 10% to about 30%.

49. The method of claim 45, wherein said chemotherapy is a camptothecin derived antineoplastic agent.

50. The method of claim 49, wherein said camptothecin derived antineoplastic agent is selected from the group consisting of camptothecin, diflomotecan, exatecan, gimatecan, irinotecan, karenitecin, lurtotecan, rubitecan, silatecan and topotecan.

51. The method of claim 50, wherein said camptothecin derived antineoplastic agent is irinotecan.

52. The method of claim 1, for treating a condition, wherein said condition is cancer.

53. The method of claim 52, wherein said cancer is selected from the group consisting of melanoma, squamous cell cancer, lung cancer, cancer of the peritoneum, hepatocellular cancer, gastric or stomach cancer, pancreatic cancer, glioblastoma, glioblastoma multiforme, KRAS mutant solid tumors, indolent non-Hodgkin's lymphoma, chronic lymphocytic leukemia (CLL), diffuse large B-cell lymphoma, thyroid cancer, non-Hodgkin's lymphoma, basal cell carcinoma, hematological tumors, B-cell non-Hodgkin's lymphoma, acute myeloid leukemia (AML), cervical cancer, ovarian cancer, liver cancer, bladder cancer, hepatoma, breast cancer, colon cancer, rectal cancer, colorectal cancer, endometrial carcinoma, salivary gland carcinoma, kidney cancer, prostate cancer, vulval cancer, thyroid cancer, hepatic carcinoma, anal carcinoma, penile carcinoma, and head and neck cancer.

54. The method of claim 53, wherein said cancer is breast cancer.

55. The method of claim 52, wherein said treating cancer comprises slowing tumor growth.

56. The method of claim 55, wherein said tumor growth is slowed by about 1% to about 30%.

57. The method of claim 55, wherein said tumor growth is slowed by about 10% to about 25%.

58. The method of claim 1, for treating a condition, wherein said condition is the presence of gut proteobacteria.

59. The method of claim 58, wherein the amount of said gut proteobacteria is reduced by about 10% to about 50% when compared to the amount present prior to administering a compound of Formula I.

60. The method of claim 58, wherein the amount of said gut proteobacteria is reduced by about 15% to about 40% when compared to the amount present prior to administering a compound of Formula I.

61. The method of claim 58, wherein said administering a compound of Formula I modulates the ratio of gut proteobacteria to the remainder of gut bacteria of at least 1:5.

62. The method of claim 58, wherein said administering a compound of Formula I modulates the ratio of gut proteobacteria to the remainder of gut bacteria of at least 1:20.

63. The method of claim 58, wherein said administering a compound of Formula I modulates the ratio of gut proteobacteria to the remainder of gut bacteria of at least 1:50.

64. The method of claim 1, wherein said condition is gastrointestinal distress caused by the treatment of a disease with an antineoplastic agent.

65. The method of claim 64, wherein said antineoplastic agent is a camptothecin derived antineoplastic agent.

66. The method of claim 65, wherein said camptothecin derived antineoplastic agent is selected from the group consisting of camptothecin, diflomotecan, exatecan, gimatecan, irinotecan, karenitecin, lurtotecan, rubitecan, silatecan and topotecan.

67. The method of claim 66, wherein said camptothecin derived antineoplastic agent is irinotecan.

68. The method of claims 45, 52, 58 or 64, further comprising administering an antineoplastic agent.

69. The method of any one of claims 1-44 or 68, wherein said administering comprises administering multiple doses or a single dose of a compound of Formula I.

70. The method of claim 69, wherein said administering comprises administration of a single dose of a compound of Formula I.

71. The method of claim 68, wherein said antineoplastic agent is irinotecan.

72. The method of claim 68, wherein said method comprises administering to a subject prior to, concurrently with, or after administration of said antineoplastic agent an effective amount of a compound of Formula I.

73. The method of claim 72, wherein said method comprises administering to a subject concurrently with said antineoplastic agent an effective amount of a compound of Formula I.

74. The method of claim 68, wherein said antineoplastic agent may be administered in a greater number of doses when compared to administering the antineoplastic agent without administering a compound of Formula I.

75. The method of claim 74, wherein said greater number of doses is in a range from about 50% to about 75% greater number of doses when compared to administering the antineoplastic agent without administering a compound of Formula I.

76. The method of claim 75, wherein said greater number of doses is in a range from about 5 to about 10 more doses of antineoplastic agent.

77. The method of claim 68, wherein said antineoplastic agent may be administered for a greater period of time without serious side effects when compared to administering the antineoplastic agent without administering a compound of Formula I.

78. The method of claim 77, wherein said greater period of time is in a range from about 50% to about 75% greater period of time.

79. The method of claim 77, wherein said greater period of time is in a range from about 5 to about 10 more days.

80. The method of claim 68, wherein said method results in an increase in survival time when compared to administering the antineoplastic agent without administering a compound of Formula I.

81. The method of claim 80, wherein said increase in survival time is in a range from about 25% to about 50% more time.

82. The method of claim 71, wherein said compound of Formula I is administered at dose between about 0.001 μg/kg and about 1000 mg/kg.
